SEQUENCE LISTING (89 sequences)

SEQ ID NO: 1 - DNA, 1167 bp, Bacteroides fragilis
atgacaatgcgaatagatacagacaaacaaatgaatttacttagtgataagaacgttgcaataattggtggtggacccgttggactgactatggcaaaattattacagcaaaacggcatagacgtttcagtttacgaaagagacaacgaccgagaggcaagaatttttggtggaacccttgacctacacaaaggttcaggtcaggaagcaatgaaaaaagcgggattgttacaaacttattatgacttagccttaccaatgggtgtaaatattgctgataaaaaaggcaatattttatccacaaaaaatgtaaagcccgaaaatcgatttgacaatcctgaaataaacagaaatgacttaagggctatcttgttgaatagtttagaaaacgacacggttatttgggatagaaaacttgttatgcttgaacctggtaagaagaagtggacactaacttttgagaataaaccgagtgaaacagcagatttggttattcttgccaatggcgggatgtccaaggtaagaaaatttgttaccgacacggaagttgaagaaacaggtactttcaatatacaagccgatattcatcaaccagagataaactgtcctggattttttcagctatgcaatggaaaccggctaatggcatctcaccaaggtaatttattatttgctaaccccaataataatggtgcattgcattttggaataagttttaaaacacctgatgaatggaaaaaccaaacgcaggtagattttcaaaacagaaatagtgtcgttgattttcttctgaaagaattttccgattgggacgaacgctacaaagaattgattcatacgacgttgtcatttgtaggattggctacacggatatttcctttagaaaagccttggaaaagcaagcgcccattacccataacaatgattggggatgccgcacatttgatgccgccttttgcagggcagggagtaaatagtgggttggtggatgccttgatattgtctgataatctagccgatggaaaatttaatagcattgaagaggctgttaaaaattatgaacagcaaatgtttatctatggcaaagaagcacaagaagaatcaactcaaaacgaaattgaaatgtttaaacccgactttacgtttcagcaattgttaaatgtataa

SEQ ID NO: 2 - DNA, 1167 bp, Artificial
  Codon Optimized for Chlamydomonas reinhardtii
atgaccatgcgcatcgacaccgacaagcagatgaacctgctgtccgacaagaacgtggcgatcatcggcggcggccccgtgggcctgaccatggccaagctgctccagcagaacggcatcgacgtgtcggtgtacgagcgcgacaacgaccgcgaggcgcgcatcttcggcggcaccctggacctgcacaagggctccggccaggaggccatgaagaaggcgggcctgctccagacgtactacgacctggccctgcccatgggcgtgaacatcgcggacaagaagggcaacatcctgagcaccaagaacgtgaagcccgagaaccgcttcgacaacccggagatcaaccgcaacgacctgcgcgccatcctgctgaacagcctggagaacgacaccgtgatctgggaccgcaagctggtgatgctggagcccggcaagaagaagtggaccctgacgttcgagaacaagccgtcggagaccgccgacctggtgatcctggcgaacggcggcatgtccaaggtgcgcaagttcgtgaccgacacggaggtggaggagaccggcacgttcaacatccaggccgacatccaccagcccgagatcaactgcccgggcttcttccagctgtgcaacggcaaccgcctgatggcgtcccaccagggcaacctgctgttcgccaaccccaacaacaacggcgcgctgcacttcggcatcagcttcaagaccccggacgagtggaagaaccagacgcaggtggacttccagaaccgcaactccgtggtggacttcctgctgaaggagttcagcgactgggacgagcgctacaaggagctgatccacaccacgctgagcttcgtgggcctggccacccgcatcttccccctggagaagccgtggaagtcgaagcgccccctgccgatcacgatgatcggcgacgccgcgcacctgatgcccccgttcgcgggccagggcgtgaacagcggcctggtggacgccctgatcctgtcggacaacctggcggacggcaagttcaacagcatcgaggaggccgtgaagaactacgagcagcagatgttcatgtacggcaaggaggcgcaggaggagtcgacgcagaacgagatcgagatgttcaagcccgacttcaccttccagcagctgctgaacgtgtga

SEQ ID NO: 3 - DNA, 1167 bp, Artificial
  Codon Optimized for Drosophila melanogaster
atgacgatgcgtattgacacagacaagcaaatgaatttgctctcggacaagaacgtcgcaataataggtggaggacctgttggtttgacaatggcaaaattgctccagcaaaacggcatcgatgtaagtgtatatgaacgcgataatgaccgggaagccaggatttttggcggtactctcgatctccacaaaggaagtggtcaggaggcgatgaaaaaggccggactgctgcagacttattatgacctggcgctcccaatgggcgtgaacatagccgataagaagggaaatatactgagcactaagaatgtgaaacctgagaatcgattcgacaaccccgagatcaaccggaatgacctccgcgcaatcctgctcaattccttggagaatgacacggtaatatgggaccggaagctcgtgatgctcgaaccaggaaaaaaaaagtggacactcacttttgagaataagccctcggagactgctgacctcgttatactggccaacggcggaatgtccaaagtacggaagttcgttacagataccgaggtcgaggaaacaggcacatttaatattcaggccgacattcaccagcccgaaatcaactgccccggtttttttcagctctgtaatggtaatcgcctcatggcttcgcaccaaggaaatctcttgttcgccaatccgaataacaacggcgcactccactttggtatatcgttcaagacgccggacgagtggaaaaatcagacccaggtagacttccagaaccggaatagcgtcgtcgatttcttgttgaaggaatttagtgactgggacgaacggtacaaagaactcattcacacgactctgtcgttcgttggattggcaactcgtatctttcccctggagaagccttggaaaagcaagcgccccttgccaataactatgatcggtgacgccgcacacttgatgccaccctttgcaggacaaggtgtaaattcgggtttggtagacgcattgattctgagtgacaacctggcggacggaaagtttaactccatagaagaagcggtcaaaaattatgagcaacaaatgttcatatatggcaaggaggcgcaggaggaaagtacccaaaatgagattgagatgttcaagccagattttacatttcagcagctgctgaatgtctaa

SEQ ID NO: 4 - DNA, 1167 bp, Artificial
  Codon Optimized for Homo Sapiens
atgacaatgcgcatagacactgacaaacagatgaatctcctttcagataaaaatgtggcaatcattggtggtggcccggtaggtcttacaatggctaaactgctccaacaaaatggcatagatgttagtgtgtacgaaagagataatgaccgcgaagcaagaatttttggaggtacacttgatcttcacaaagggtctggtcaggaagcgatgaaaaaagctggtttgttgcaaacttactatgacctcgcgttgccgatgggtgtcaacattgccgacaagaagggcaacatcctctctacgaaaaatgttaagccggaaaacaggttcgacaatcccgaaatcaaccggaacgaccttcgcgcgatactcttgaattcccttgagaatgacacagtcatctgggaccggaaactcgttatgttggaacctggcaaaaaaaagtggacgttgacgttcgagaataagccctctgagactgcagaccttgttatacttgccaatggaggcatgagtaaagtcagaaagttcgtcaccgatacggaagtagaggagaccggcacgtttaatatacaagcagatatccatcaacccgagatcaattgccccggtttctttcaactttgcaatggtaatcggctgatggcaagccatcaggggaatctcctctttgccaatcctaacaacaacggggcgcttcatttcggtataagcttcaagacccccgatgaatggaagaaccagacgcaggttgattttcaaaataggaatagcgttgtggactttcttctgaaagagttttctgattgggatgaacggtataaagaacttattcacacgactctttcctttgttgggctggccacaagaatctttcctctggagaagccttggaaatctaaacgacctcttcctataactatgatcggtgacgctgcgcatttgatgccaccgttcgctggtcaaggcgtaaactccgggctcgtagacgctctgattttgtccgataatctcgctgatggtaagttcaattcaatagaagaagctgttaaaaactacgaacaacaaatgtttatatacggcaaggaggcccaggaggaaagcactcaaaatgagatagaaatgtttaagccagacttcactttccaacagctgcttaacgtataa

SEQ ID NO: 5 - DNA, 1167 bp, Artificial
  Codon Optimized for Mus Musculus
atgactatgaggatagacaccgataagcagatgaacctgctgtccgacaagaacgtggctattatagggggaggtcctgtaggtctcacaatggcaaaactccttcagcaaaatgggatcgacgtaagtgtatatgagcgggataatgatagagaggctcgaatctttggaggcaccctcgacctccacaaaggatcagggcaagaggcaatgaagaaagcaggattgctgcaaacatattatgacctcgcactcccaatgggtgtgaatatcgcagataagaaggggaatatcctcagtacaaaaaatgttaagccagagaataggtttgataatcccgagattaaccgaaacgacttgcgcgcaatactgcttaactctttggaaaatgataccgtcatatgggaccgcaaactcgtcatgctcgagcccggtaagaagaagtggactttgacatttgaaaacaaaccatccgaaaccgctgatctggttatacttgctaatggcggaatgtccaaggtaaggaagttcgtgaccgacactgaggtggaagagacaggaacatttaatatccaggcagacatccaccagccagaaataaactgtcctggcttcttccagttgtgtaatgggaatagactcatggcctcacaccaaggaaatcttcttttcgccaaccccaataataatggtgctctccacttcggcatatcattcaaaactccagatgagtggaaaaaccagactcaagttgatttccagaacagaaattcagtagtcgactttcttttgaaagagttctcagactgggacgaaagatacaaggaactcatacacacaaccctttcatttgtcggtctggcaactaggatctttccactggagaagccttggaagtcaaagaggcctcttccaattactatgatcggtgatgccgctcatctgatgcccccattcgcaggccagggcgtaaattccggcctggttgacgcactgattctctcagataatctggccgatggcaaatttaactccatagaggaggcagtcaaaaactatgagcaacagatgttcatatacgggaaagaagcccaggaagaaagcacacagaatgagatagaaatgttcaagcctgattttacttttcagcagctgttgaatgtgtaa

SEQ ID NO: 6 - DNA, 1167 bp, Artificial
  Codon Optimized for Pichia Pastoris
atgacaatgcgtatagacacagacaagcaaatgaacctattatctgataaaaatgtagccataattggtggaggtcctgtgggccttactatggcaaagctgctgcaacaaaacggcatagacgtgtccgtgtatgagagagacaacgatcgtgaagccaggattttcggaggcacccttgatctacataagggatccggccaagaggctatgaagaaggctggcttgctgcagacatactacgaccttgctctgcctatgggagtgaatatagccgacaagaaaggcaacattctatccacgaagaacgttaaaccagagaacagatttgataatccagaaatcaacagaaacgacttgagggccatcctgttaaactccctggagaatgacaccgtaatttgggaccgtaagcttgtgatgttggaacctggaaaaaagaaatggactcttactttcgagaacaagccctcagagaccgcagatctggtcattctggctaatggcggaatgagtaaagttcgtaaatttgttactgatacggaagtagaagaaacaggcacgttcaacattcaggcagatatacatcagcccgaaataaattgtccaggatttttccagctgtgtaatggcaatagattaatggcctcccatcaaggaaatttgttattcgccaacccaaataacaatggtgccctgcatttcggtatttcatttaaaactccagacgaatggaagaatcaaacccaagttgatttccagaataggaactcagtagtagactttcttcttaaggagttcagtgactgggatgagaggtataaggagcttattcatacgacactgtctttcgtgggtcttgcaacaaggattttccctctggagaagccttggaaatcaaagaggcctttgccaatcaccatgatcggcgatgctgctcatttgatgccccctttcgctggtcaaggtgttaactccggtttggtggatgctcttatcctatccgacaacctagcagatggaaagtttaattccatagaggaagccgttaagaactacgaacagcaaatgttcatatatggtaaggaggctcaggaggagtctacacaaaatgaaatagagatgttcaaacctgactttacgttccagcagttgttgaacgtttaa

SEQ ID NO: 7 - DNA, 1188 bp, Artificial
  Codon optimized for yeast, for example, S. cerevisiae
atgactatgcgtattgacactgacaaacaaatgaacttattatctgacaaaaatgttgctattattggaggtggtcctgttggattgactatggctaagttgttgcaacaaaacggtattgatgtttctgtttacgaaagagataacgatagagaagctagaattttcggtggtactttagatttgcataagggttcaggtcaagaagctatgaagaaagctggtttgttgcaaacttactacgatttggctttgccaatgggtgttaacattgctgataagaaaggaaacattttgtctactaaaaatgttaagccagaaaacagattcgataaccctgaaattaatagaaatgatttgagagctattttgttgaactcattagaaaacgatactgttatttgggatagaaaattagttatgttggaaccaggtaaaaagaagtggactttgactttcgaaaataagccttctgaaactgctgatttggttattttggctaatggtggaatgtcaaaagttagaaagttcgttactgatactgaagttgaagaaactggtacttttaatattcaagctgatattcaccaaccagaaattaattgtcctggtttctttcaattgtgtaacggtaacagattgatggcttctcatcaaggaaatttgttgttcgctaacccaaacaacaatggtgctttgcactttggtatttcttttaagactcctgatgaatggaagaatcaaactcaagttgatttccaaaacagaaactctgttgttgattttcttttgaaggaattttcagattgggatgaaagatacaaggaattaattcatactactttgtctttcgttggtttggctactagaattttcccattggaaaaaccttggaaatcaaagagaccattacctattactatgattggagatgctgctcacttaatgccaccttttgctggtcaaggtgttaactctggtttggttgatgctttgattttgtcagataatttggctgatggaaagtttaattctattgaagaagctgttaagaattacgaacaacaaatgttcatctatggtaaagaagctcaagaagaatcaactcaaaatgaaattgaaatgtttaaaccagacttcactttccaacaacttcttaacgtacatcaccatcaccatcactaataa

SEQ ID NO: 8 - DNA, 1167 bp, Artificial
  Codon Optimized for Arabidopsis Thaliana
atgactatgcgaatagatacagataagcagatgaaccttcttagtgataagaacgtcgcgataataggcgggggtccggtaggtctcaccatggcgaaactgctacaacaaaatggtattgatgtctctgtgtatgagagggataacgatagagaggccaggatatttggagggaccctcgatcttcacaaaggcagcggccaagaagctatgaagaaggcagggctcctacaaacttactatgacctcgcactcccaatgggtgttaacattgcagataagaagggaaacattttatccaccaagaacgtcaagcccgagaatcgttttgataacccggagattaaccgtaatgatctacgtgcaattctgcttaattccctggaaaacgatacagtaatctgggatcgtaaattggtcatgctagagcccggaaagaaaaagtggacattaaccttcgagaacaagcccagcgagacggctgacttggttatactggctaatggcggcatgtccaaggtaagaaaattcgttacggacaccgaagtagaggaaacgggcacgtttaacattcaggctgatatccaccagccggaaattaattgcccgggatttttccagctatgcaatgggaacagactgatggcgtcccatcaaggaaatttgctatttgccaatccgaacaacaatggtgctctgcacttcggcatatcctttaagactcctgatgaatggaagaaccaaactcaagttgattttcaaaaccgaaactcagtcgttgattttcttctcaaagaattctcagattgggatgagcgatacaaagaacttatccatactacactcagttttgttggactagcgacgcgaatattccctcttgaaaagccctggaaatccaagcgtcctctaccaattactatgattggggacgcagctcatctgatgccgccctttgccgggcaaggtgttaatagtggcctagtagacgcattgatattatccgacaatctagccgacggcaagttcaattctatcgaggaggcagtgaagaactatgagcagcaaatgtttatttacggtaaggaggctcaagaagagagtacgcaaaatgagattgagatgttcaaacccgacttcacgtttcagcaactcctcaacgtataa

SEQ ID NO: 9 - DNA, 1167 bp, Artificial
  Codon Optimized for Aspergillus Niger
atgactatgcggattgacacagataaacaaatgaatttgttgtcagacaaaaatgttgcgattattggaggcggaccggtcggtctcaccatggccaaactgttgcagcaaaatggtattgatgtgtcagtctacgagagagacaatgaccgagaagcacgtatttttggaggaacacttgacctgcacaaagggagcgggcaggaagcaatgaagaaggccggactccttcaaacgtattatgacctggcattgcccatgggtgtaaatattgccgataagaaagggaacatcctcagtaccaagaatgtgaaacctgagaacagattcgacaacccggagatcaataggaatgaccttagagcaattcttctcaatagtcttgagaatgatacagtgatatgggaccgcaagctcgtcatgctcgaaccgggcaagaaaaaatggacgctcactttcgagaacaaaccatcggagacagctgatctcgtaatcctcgcaaatggaggaatgtcaaaagttcgcaaatttgtaacggacactgaagtggaggaaacaggaacctttaatattcaggcggatatacaccaaccggagataaattgtcctgggttcttccaactgtgcaatggaaacagactgatggcttcccatcagggaaatctgctctttgcgaacccaaacaacaatggagctctgcacttcggaatatctttcaagacgcccgacgagtggaagaatcagacccaggtggatttccaaaaccggaattctgtagtagatttcctgctgaaagagttttcggattgggacgaaagatataaagagttgatccatacaacactgtcctttgtgggactcgcaacgagaatattccctctggagaagccgtggaagtccaaacggcccctgcctataactatgataggggacgcggcgcacctgatgcctccgtttgctggccagggggttaactctggcctggttgatgcactcatactgtccgataaccttgctgacggcaaatttaattccatagaggaggcggtcaagaactacgaacagcagatgttcatctatggaaaggaagcacaggaagaatcgactcaaaatgaaattgagatgtttaagccagacttcaccttccagcaactcttgaatgtgtaa

SEQ ID NO: 10 - DNA, 1167 bp, Artificial
  Codon Optimized for Bombyx Mori
atgactatgcgtattgataccgataaacagatgaacctactgagcgataagaacgtagcgatcataggtggtggaccagtaggtctcactatggcaaaactcttacagcagaatggtatcgacgttagtgtatatgagcgagacaacgacagggaggctcgtattttcggaggtacactggatttacataagggatcaggccaggaagctatgaaaaaagcggggcttctacaaacctactacgatttagctcttcccatgggggtgaatattgcagataagaaaggaaatatcctctcaacaaaaaatgttaaaccggagaacaggtttgacaatccggagataaatagaaacgacttacgagcgattctattaaacagcttggagaacgataccgtaatctgggaccgcaaacttgtgatgttggaaccaggaaaaaaaaagtggacactcaccttcgagaacaaacccagcgagacagcggacctcgtgatactcgctaacggaggcatgagtaaggtacgcaagtttgtaaccgacacagaggtagaggaaactggcacgtttaacatccaggccgatatccaccagcctgagataaactgtccgggcttttttcagctgtgtaatgggaacagattaatggcctcacatcaaggaaatttgttattcgcaaacccgaataataacggggccttgcattttggaatctcattcaaaactccggacgagtggaaaaatcagacccaggtcgattttcaaaacagaaactccgtggtcgatttcttgctcaaggaattttcagattgggacgaacgatataaggaactaatacataccactctttcctttgtcggtttagcgacgaggatctttcctttagagaaaccatggaagagtaaacgtcctcttccgattacgatgattggggacgccgcccatctgatgccgccatttgcggggcagggagtaaatagcgggctcgtggatgcgttgatactgtcagataatttagctgatggtaaatttaatagcatagaagaagccgttaagaattatgaacagcaaatgttcatctacggcaaagaagcgcaggaggagtccactcaaaacgaaatcgaaatgtttaaaccagactttactttccaacagctactaaacgtctaa

SEQ ID NO: 11 - DNA, 1167 bp, Artificial
  Codon Optimized for Bos Taurus
atgaccatgaggattgatactgataagcaaatgaatctcctgtcagacaagaatgttgcaattatcggtggaggcccggttggtcttacgatggctaagctgcttcagcagaacgggatagatgtgagtgtgtatgaacgcgacaacgatcgggaagcgaggatattcggggggacacttgatcttcataagggctctggacaggaggctatgaaaaaggccggtctgctccaaacgtactatgatttggcccttccaatgggagtcaatatcgcagacaaaaagggtaacattctttccacaaagaatgttaagcccgaaaataggtttgataacccagagatcaaccgcaatgacttgcgagcgatattgctcaattcattggagaatgatactgttatttgggaccgaaaattggttatgcttgagccgggtaaaaagaaatggacgttgacgttcgagaacaagccttccgagacggctgacttggtcatactggcaaacggaggtatgtctaaagttaggaaatttgttactgatacagaggtggaggagaccggaactttcaatatccaagctgatattcatcagccagagataaactgccccgggttcttccaactctgtaacggaaacaggctcatggcctcacaccagggtaatcttctttttgcaaatcccaacaacaatggagcccttcattttgggatctcatttaagacccccgatgaatggaaaaaccaaactcaggtggatttccagaaccggaacagcgttgttgattttctgctcaaagagttttctgattgggacgaaagatataaagagctcatccatacgactctctctttcgtcggtcttgctacgcgaatctttcccctggaaaagccctggaaaagtaagaggcctttgcccatcaccatgataggagatgccgcccatctgatgcctccgtttgcggggcagggagttaatagcggtttggtggacgcccttatcctgtctgacaatcttgcggacggtaagtttaacagcatcgaagaggccgtgaagaactatgagcagcagatgtttatctacggcaaggaggcgcaagaagaatcaacacaaaatgagatagagatgttcaagccagatttcacatttcaacagcttcttaacgtgtaa

SEQ ID NO: 12 - DNA, 1167 bp, Artificial
  Codon Optimized for Danio Rerio
atgacaatgagaatagacacagacaaacagatgaacttgttgtctgacaaaaacgtggccatcattggagggggacccgtcggcctgacgatggccaagttgctgcaacagaatggaatagatgtgtctgtgtacgaaagagataatgaccgggaggctcgtatctttgggggcacactcgatcttcacaaaggttctggacaagaagccatgaagaaggctggtctgctgcaaacgtactacgatctcgcattgccaatgggggtaaacatagcggataaaaaaggaaatattctttcaacgaagaatgttaagccggaaaacagattcgataaccccgaaataaacaggaatgatctcagggccatcctcctcaactctcttgagaacgacactgtcatctgggaccggaaactcgtgatgctggaaccgggcaaaaagaaatggacgttgacatttgagaacaaaccttcagagacagccgacttggttattctggcaaatggtggtatgtccaaggttcggaagtttgtgacggacacggaggtagaggaaaccggaacatttaatatccaggcagacatacatcagcctgagatcaactgcccaggatttttccaattgtgtaacggaaatcgtttgatggcgtcccatcaaggcaatcttttgtttgcaaatccgaataacaatggggccctgcatttcggaatctcatttaagactccagatgagtggaagaatcaaacacaagtcgatttccaaaaccgtaattccgttgtagactttttgctcaaggaatttagtgattgggatgaaagatataaagaattgattcataccaccctgagtttcgtcggactggctactcgtatctttcctcttgaaaaaccctggaaaagcaagcgacctttgccgataactatgataggtgatgcagcacaccttatgccgccctttgctggtcaaggagtgaacagcggtctcgttgacgctcttattttgagtgacaatttggccgatggtaagttcaacagtattgaagaagctgttaaaaactacgaacagcaaatgttcatatatggaaaggaggcgcaggaggagtcaacccagaacgagatcgagatgttcaaacctgactttacatttcagcaactcctgaacgtttaa

SEQ ID NO: 13 - DNA, 1167 bp, Artificial
  Codon Optimized for Brassica Napus
atgactatgagaatcgacactgacaagcagatgaacctgttgagcgataagaacgtcgctataattggtggcgggccagttggcttgacgatggcgaagttgttgcagcagaatggaattgatgtatccgtgtatgagagggacaacgacagagaagctcgtatcttcgggggaaccttggacttgcataagggcagcggccaagaggcgatgaagaaagctggcttgcttcagacgtattatgatctcgcgctgccaatgggcgtcaacatcgccgacaaaaaaggcaatatcttgtccacaaagaatgtgaagccggaaaacagattcgataatccagaaataaacagaaacgaccttcgtgctatactgctgaatagtctcgagaacgacactgtgatttgggaccgtaagctggttatgttggagcctggaaagaagaagtggaccttgacgtttgaaaataagccgagtgaaactgcggatttggttattcttgccaatggcggaatgtcgaaggtgaggaagtttgtcacggacacagaagtcgaggaaactggcacatttaatatacaagccgacatccatcagcctgaaatcaattgtccaggcttttttcaactttgtaacggtaaccgtctgatggcatcgcaccagggcaacctgctttttgcaaacccaaataacaacggtgccctgcacttcggtatctctttcaaaacaccagatgaatggaaaaaccagactcaggtggattttcaaaataggaacagcgttgtagattttcttctgaaggaattctctgactgggatgaaaggtataaagagttgattcatacaacactcagcttcgtaggactggcgaccaggatatttcccctcgaaaagccatggaagtccaaaaggcccctgcccattacaatgatcggtgatgcggctcaccttatgccccccttcgctgggcaaggggtgaactccgggcttgttgacgcgcttatcctttcggataacttggcagatggtaaatttaattccatcgaggaggcagtcaagaattacgagcagcaaatgtttatatacggcaaagaagcacaggaagaatctacgcagaacgagattgaaatgtttaagcccgatttcacttttcagcaactgctcaatgtctaa

SEQ ID NO: 14 - DNA, 1167 bp, Artificial
  Codon Optimized for Caenorhabditis Elegans
atgactatgcgtattgatactgacaagcagatgaacttactgtcagacaaaaacgtcgcaattatcggaggaggaccggtaggccttacgatggcgaagcttttgcaacagaacggcattgatgttagtgtatacgaacgtgacaatgatagagaagcccgtatatttggaggaaccctggacctccacaagggctcaggacaggaagctatgaaaaaagccggattactgcaaacgtactacgatctggcgctgccaatgggcgtgaatatagcggataaaaagggcaacatactttcgacaaaaaatgtaaagcctgaaaatcgttttgataacccagagataaatcgtaacgacctccgtgcaattttattgaactctcttgagaatgatacggttatctgggaccgtaagttggtaatgcttgagccgggtaaaaagaaatggacattaacattcgagaacaagccttctgagacggccgatttggtcatattggccaacggaggaatgtcaaaagtacgaaagttcgtcaccgatacagaggtagaggaaacaggcacttttaacatacaggctgatatccaccagccggagattaactgcccaggattttttcagttgtgtaacggtaatcgtctgatggcctctcaccaaggcaatcttcttttcgctaatcctaataataacggcgcgttacatttcggtataagtttcaagactccggatgaatggaagaaccaaacccaggtggatttccaaaaccgaaactcagtagtcgactttctgctcaaggagttctctgattgggacgagcgatacaaagagttaatacacactacactctcgtttgtgggtctggcgaccagaatatttcctctcgagaagccttggaaatccaaacgaccactcccaataacgatgataggcgacgccgcccacctcatgccaccattcgccggccaaggtgttaattctggccttgtagacgctcttatactgtccgacaatttagccgacggtaaattcaattcgattgaggaagctgttaagaattacgagcaacagatgttcatttacggcaaggaggctcaggaggagtcaacccagaatgaaatagagatgtttaaaccggacttcacttttcagcagctgctgaatgtgtaa

SEQ ID NO: 15 - DNA, 1167 bp, Artificial
  Codon Optimized for Candida Albicans
atgactatgcgaatcgatacagacaaacaaatgaacttattatcagacaagaatgtagcaataattggagggggtcctgtcggacttacaatggctaaacttcttcaacaaaacggaatcgacgttagtgtgtacgagcgtgataatgatagagaggcaagaatattcggaggaacccttgacttacacaaggggtcaggacaagaagcaatgaaaaaggctgggttattgcaaacctattatgatcttgccttgcctatgggtgtgaacatcgccgacaagaaaggtaatatcctttccactaaaaatgtaaaaccagaaaacagatttgataatcctgaaataaatcgtaatgatcttcgtgctatcttacttaattcattagaaaacgatacagtgatttgggatcgtaaattggtgatgcttgaaccaggaaagaaaaaatggacccttactttcgagaataagccaagtgaaactgccgacttggtgatccttgccaacggtggaatgtctaaggtcagaaagtttgtgaccgatactgaggtggaagagactgggacattcaatatccaggccgatatacaccagcctgaaataaactgcccaggattctttcaattatgcaacgggaaccgacttatggctagtcatcaggggaatttgttgttcgcaaatccaaataataatggtgcacttcattttggaatatcctttaagacccctgacgaatggaaaaaccagactcaggtggattttcagaatcgtaactctgtagtcgactttcttttaaaggaattctctgactgggacgagcgatacaaagagttgatacacactacattgtcattcgttgggttggcaactcgaatatttcctcttgagaaaccttggaagtctaaacgtccattgcctataaccatgataggagacgcagcccacttgatgccaccatttgccggacaaggtgtcaattctgggcttgttgacgcccttatcttatccgacaatttggcagatgggaaattcaactcaattgaagaagctgtgaaaaattatgagcagcagatgttcatttacgggaaggaagcccaggaagagagtacccaaaatgagatagagatgtttaagcctgactttaccttccagcagttgttgaatgtataa

SEQ ID NO: 16 - DNA, 1167 bp, Artificial
  Codon Optimized for Canis Familiaris
atgacgatgaggatagatactgacaaacaaatgaacctcctgagcgacaaaaatgtggcaatcattggaggagggccggtcggacttacgatggctaaactccttcaacagaacggcatagatgtttccgtttatgagagggataatgacagggaagctagaatttttggcggaacccttgatcttcacaagggaagcgggcaagaggctatgaaaaaagctggcttgctgcaaacctattatgaccttgcactccctatgggtgtgaacattgcagataagaaaggaaacatcctctctaccaagaacgtcaaaccagagaatagatttgataacccagagatcaatcggaatgatctgcgggcaatactgttgaactcactcgaaaacgacactgtcatttgggaccgaaagctggttatgctggagcccgggaagaaaaaatggacgctcacttttgagaacaaaccttcagaaaccgcggacctcgtcattcttgcgaacggaggtatgtcaaaagttcggaagttcgtcactgatactgaggtggaagagactggtacatttaacatacaagctgatatacatcaaccagaaattaactgccccggctttttccaactgtgcaacggcaataggctcatggcatcacatcagggcaatttgctcttcgccaacccaaataataacggggcacttcacttcggcatctcctttaaaactccggacgaatggaaaaatcagacccaggtggacttccaaaatcgcaacagtgttgttgattttctccttaaggagtttagcgattgggatgaaagatataaggagttgatacacaccacattgtcattcgtgggcctcgctactcgaatattccctctggaaaaaccatggaagagtaagagacccctcccgattactatgattggagacgccgctcatttgatgccgccgtttgccggccagggggttaattccggcttggttgacgctctcattttgagcgataatctcgcagatgggaaattcaactcaatagaagaagcagttaaaaattatgagcaacaaatgttcatatatggcaaagaagcgcaggaagaatcaactcaaaacgaaattgaaatgttcaagcctgactttacattccagcaactccttaatgtgtaa

SEQ ID NO: 17 - DNA, 1167 bp, Artificial
  Codon Optimized for Chlamydomonas Reinhardtii
atgaccatgcgcatcgataccgataagcagatgaacctcctgtccgacaagaacgtcgcgattattgggggtggcccggtcgggctcaccatggctaagctcctgcagcagaacggcattgacgtgtcggtgtacgagcgcgataacgaccgcgaggcccgcatttttggtggtacgctcgatctgcataagggctccggccaggaggctatgaagaaggccggtctcctccagacgtactacgacctcgccctgcctatgggggtgaacatcgctgataagaaggggaacatcctgagcacgaagaacgtcaagccggagaaccgcttcgataacccggagatcaaccgcaacgacctccgggcgatcctgctcaacagcctcgagaacgacaccgtgatttgggaccggaagctcgtgatgctcgagcccggcaagaagaagtggacgctcacgttcgagaacaagccctccgagacggccgatctggtgattctggctaacggcggcatgagcaaggtgcgcaagtttgtcacggacaccgaggtcgaggagacgggtacgttcaacatccaggcggatattcatcagccggagattaactgcccgggctttttccagctctgcaacgggaaccggctgatggcgtcgcatcagggcaacctgctctttgctaaccctaacaacaacggtgcgctccactttgggatctccttcaagaccccggacgagtggaagaaccagacgcaggtcgatttccagaaccgcaactccgtggtggatttcctcctgaaggagttttccgattgggacgagcggtacaaggagctgatccatacgaccctgtcgttcgtgggcctggcgacgcgcatctttcccctcgagaagccttggaagtccaagcggcctctgcctatcacgatgatcggtgacgcggctcacctgatgccgccgtttgctggccagggtgtgaactcgggcctggtggacgctctgattctcagcgacaacctcgccgacggcaagttcaactcgattgaggaggccgtgaagaactacgagcagcagatgttcatctacggcaaggaggcccaggaggagagcacccagaacgagatcgagatgttcaagcccgacttcacgttccagcagctcctcaacgtctaa

SEQ ID NO: 18 - DNA, 1167 bp, Artificial
  Codon optimized for the cells of Cricetulus griseus
atgaccatgcgcatcgacacagataaacagatgaatctgctgtccgacaagaacgtggccatcattggcggagggccagtcggactgacaatggccaagctgctgcagcagaatgggattgatgtgagtgtctacgagcgagacaacgatagggaagctcggatctttggtggcaccctggacctgcacaagggttcaggccaggaggcaatgaagaaagccggactgctgcagacatactatgatctggcactgcctatgggcgtgaacatcgccgacaagaaaggcaatattctgtctaccaaaaacgtgaagcctgagaatagattcgacaacccagaaattaatcgaaacgatctgcgtgccatcctgctgaatagtctggagaacgacactgtgatctgggatcgcaaactggtcatgctggaaccaggcaagaaaaagtggactctgacctttgagaataagccctccgaaaccgccgacctggtcatcctggcaaacggagggatgagcaaagtgcgaaagttcgtcacagatactgaggtggaggaaaccggcaccttcaacatccaggctgacattcatcagccagaaatcaactgccccggattctttcagctgtgcaatgggaaccgtctgatggcaagccaccagggcaatctgctgttcgccaaccctaacaataacggagctctgcatttcgggatctcttttaaaacaccagacgagtggaagaaccagactcaggtggattttcagaataggaacagtgtggtggacttcctgctgaaggaattttcagactgggatgagcggtataaggaactgatccacaccacactgtctttcgtgggactggccacaagaatttttcccctggagaagccttggaagagtaagcgccctctgccaatcactatgattggggatgccgctcatctgatgccccctttcgctggacagggggtgaattcaggtctggtggacgctctgatcctgtccgacaatctggcagatggcaaattcaactccattgaggaagcagtgaagaactacgagcagcagatgtttatctatggcaaagaagcccaggaggaaagcactcagaatgagatcgaaatgtttaagcctgacttcacctttcagcagctgctgaacgtgtga

SEQ ID NO: 19 - DNA, 1167 bp, Artificial
  Codon Optimized for Cyanophora Paradoxa
atgacgatgcgtattgacaccgataagcagatgaacctgctgagcgataagaacgtggctattattggcggcggtcccgtgggtctgacgatggcgaagctcctgcagcagaacggcatcgacgtttccgtctacgagcgtgacaacgatcgtgaggcccgtatcttcggtggtacgctcgatctccacaagggtagcggccaggaggccatgaagaaggccggtctcctccagacttactacgatctcgccctgccgatgggtgttaacattgctgataagaagggcaacattctcagcaccaagaacgtcaagccggagaaccgcttcgacaacccggagatcaaccgtaacgacctgcgcgctattctcctcaactcgctggagaacgatacggttatttgggatcggaagctggttatgctggagcccggtaagaagaagtggaccctcacgttcgagaacaagccgagcgagaccgctgatctcgttattctcgctaacggcggtatgagcaaggtccgtaagttcgtcaccgatacggaggttgaggagaccggcacgttcaacatccaggccgacattcaccagccggagatcaactgccccggcttcttccagctctgcaacggtaaccggctcatggcctcgcaccagggtaacctgctgttcgcgaacccgaacaacaacggcgcgctccacttcggcatctccttcaagacccccgacgagtggaagaaccagacccaggttgacttccagaaccgtaacagcgttgtcgatttcctcctgaaggagttctccgattgggacgagcgctacaaggagctgatccacaccaccctctcgttcgtcggtctggctacgcgcatcttcccgctggagaagccgtggaagagcaagcgtccgctccccatcactatgatcggcgacgcggcccacctgatgccgccgttcgcgggtcagggtgttaacagcggcctcgtggatgcgctgattctcagcgacaacctcgcggacggtaagttcaactccatcgaggaggctgtcaagaactacgagcagcagatgttcatttacggtaaggaggcccaggaggagagcacccagaacgagattgagatgttcaagccggacttcacgttccagcagctcctcaacgtgtaa

SEQ ID NO: 20 - DNA, 1167 bp, Artificial
  Codon Optimized for Dictostelium Discoideum
atgaccatgcgtatagataccgataaacaaatgaatttgttgagtgataaaaatgtagccataatcggaggaggacctgtaggtttgaccatggctaaattgttacaacaaaatggtatcgatgtaagtgtatacgagcgtgataatgatagagaggcaagaatcttcggaggaacattggatttgcacaaaggatctggacaagaagcaatgaaaaaagccggacttttgcaaacctattacgatcttgcccttccaatgggtgtaaatatagcagataagaagggaaatattttatctactaaaaatgtaaaacctgagaatagattcgataatcctgagatcaatcgtaatgatttaagagccatattgttgaattctttggagaatgatactgtaatctgggatcgtaaacttgttatgttagagcctggaaaaaaaaagtggacacttaccttcgaaaataagccttcagagacagccgatttggtaatccttgctaatggaggaatgagtaaagttcgtaaatttgtaactgatactgaagttgaggaaactggaacttttaatattcaagcagatattcatcaacctgagattaattgtccaggtttttttcaattatgtaatggaaatcgtttgatggcctcacaccaaggtaatttattgttcgctaatccaaataataatggagcacttcatttcggtatctcttttaagacccctgatgaatggaagaatcaaacacaagtagattttcaaaatcgtaattcagtagttgattttttattaaaagagttctcagattgggatgaaagatacaaagaattaatccataccactttgagtttcgttggacttgctacaagaatcttcccattggagaagccttggaaatcaaagcgtcctttgccaatcaccatgattggtgatgcagcacacttgatgcctccattcgcaggacaaggtgttaattcaggtttggtagatgctttaatcttatcagataatcttgctgatggtaagtttaatagtatcgaggaagccgtaaaaaattatgagcaacaaatgtttatctatggtaaagaggctcaagaagaatctacccaaaatgagatcgagatgttcaaacctgatttcacttttcaacaattattaaatgtataa

SEQ ID NO: 21 - DNA, 1167 bp, Artificial
  Codon Optimized for Emericella Nidulans
atgactatgcggatagatacggataagcaaatgaacctgctctcagacaagaacgtcgcaatcattggaggaggacccgtcggtttgactatggccaaacttcttcaacaaaacggcatcgacgtctctgtttatgagcgtgacaatgaccgtgaggcgcgtatattcgggggaacgcttgatctgcataaaggtagcggacaagaagcgatgaaaaaggccggattgctccagacatattatgacttggcgctccctatgggagtgaacatcgccgataagaagggaaacattctcagcacaaaaaacgtgaaacccgagaatcggtttgacaatccagagataaatcggaacgatctccgagcaatcctgcttaactctttggaaaatgatactgtgatctgggaccggaagcttgttatgttggaaccaggaaagaagaagtggacgctcacgttcgagaataaaccgagtgagactgccgacttggtgatcctcgccaatggcggtatgagcaaagtccgcaaattcgtcacggataccgaagtcgaagaaacaggtacgtttaatatacaagcagacattcatcagcctgagattaactgccccggtttttttcagctctgtaatggaaaccgccttatggcttctcaccagggaaatctcctctttgcgaaccccaacaataatggagcattgcacttcggaatttcatttaaaactcccgatgaatggaagaaccagacccaggttgacttccaaaatcgaaactcagttgttgactttctgctgaaggaattctccgactgggatgagcgctacaaggagctcattcacactacgctctcctttgtcggattggcaacccgtatttttcctctggaaaaaccatggaaaagcaagcgtcctcttcccataacgatgataggagatgcggctcacttgatgccaccattcgctgggcagggggtgaatagtggtcttgttgatgctcttatcctctctgacaatctggccgacggaaaattcaattcgatagaggaagctgtcaaaaactacgagcaacaaatgttcatatacggaaaggaggcccaggaggaaagcacccaaaatgagattgagatgttcaagcctgacttcactttccagcagttgctcaacgtgtaa

SEQ ID NO: 22 - DNA, 1167 bp, Artificial
  Codon Optimized for Gallus Gallus
atgaccatgcggatcgacactgataaacaaatgaatcttctcagtgataaaaacgtggctatcataggcggaggccccgtgggattgacaatggcgaagttgctgcaacagaacggaatagatgtgagcgtgtatgagagagacaacgatcgcgaggcgagaattttcggcggcactcttgacctccataaggggtcaggccaggaggcgatgaagaaggctggtcttttgcaaacttattatgatctggctttgccaatgggagtgaacatagccgacaagaaaggcaacatactcagcaccaaaaatgtcaaaccagagaatagatttgataacccagaaatcaatagaaacgacctgagggcaatcctgctgaactctttggagaatgatacggttatttgggacaggaaattggtgatgttggagcctgggaaaaaaaaatggacacttacgttcgagaataaaccgtctgagacggcggacctcgtcatcttggcaaatggtggcatgtccaaagtcagaaaattcgttactgatactgaagtcgaagaaacagggacatttaacatccaagcagacatccaccagcccgagatcaactgtccgggattcttccagctttgcaatggcaacagacttatggcttctcaccaggggaatctgctttttgcgaacccgaacaacaatggggctctccactttggtattagttttaagacaccggatgagtggaagaatcaaactcaagttgactttcaaaacagaaattccgtcgtagacttcttgctgaaggaattttctgactgggacgaacgctataaggagcttatacatacgactctgtctttcgtcggtttggcaacacggatcttccctcttgaaaagccctggaagtccaagagacctctccccatcaccatgatcggtgatgccgcacatctcatgcctccctttgccggacagggcgtgaactccggtcttgtagatgccttgattctctctgataatctcgccgatgggaaattcaattcaatcgaagaagctgtcaaaaattatgaacaacagatgtttatctatggcaaggaagcccaagaagaatccacccagaatgagatcgaaatgtttaaacctgatttcactttccagcaactcctgaacgtttaa

SEQ ID NO: 23 - DNA, 1167 bp, Artificial
  Codon Optimized for Glycine Max
atgaccatgagaatcgacacagacaagcagatgaatctcctttcagacaaaaacgtcgcaataataggcggaggacctgttgggctgaccatggctaaactcttgcagcaaaatggcatcgacgtatccgtgtatgagcgtgacaatgatagagaagcaagaatctttggagggactttggatctccacaaggggtcaggccaagaggcaatgaagaaagctgggcttttgcaaacctattacgacctcgccctccccatgggcgttaatatagcagataagaaagggaatattctcagcactaagaacgtcaagcccgaaaataggtttgacaaccctgagatcaacagaaacgaccttagggctattctcctcaattcattggaaaacgatactgtcatttgggatcgcaaattggtaatgctcgaacccggtaaaaagaagtggactctgacttttgaaaataagccatctgagactgcagacctggtcattctcgctaatggcggtatgtcaaaggtacgtaaattcgtgactgacactgaggtcgaagaaaccgggactttcaacatacaggccgatatccatcagcccgaaataaactgtcccggtttcttccagctctgcaacggtaatcgccttatggcatctcaccaaggaaacctgttgttcgcaaatcctaataacaatggggctctgcatttcgggatttcatttaagactcctgatgaatggaaaaaccagacccaggtggattttcaaaacagaaacagtgtagtggattttcttttgaaagaattttccgattgggatgagaggtacaaagagttgatccacacaaccctctcattcgtaggcctggcaacacgtatttttccactggaaaaaccttggaagagcaaaagaccactccctatcaccatgatcggtgacgcagctcatctcatgcctcctttcgccggccagggggtcaattctggtctcgtcgatgcattgatactgagcgacaatctcgcagatggaaaatttaattctatagaagaagctgtcaaaaactatgaacaacaaatgttcatctatggaaaggaggctcaggaagaatcaactcaaaacgagatagaaatgtttaagcccgactttacctttcagcaactcctcaacgtctaa

SEQ ID NO: 24 - DNA, 1167 bp, Artificial
  Codon Optimized for Hordeum Vulgare subsp Vulgare
atgacgatgaggattgatactgacaagcaaatgaatcttctgagcgacaagaacgtggcaataattggaggtggccccgtcggactcactatggcgaaactcttgcagcaaaacggcatcgatgtctcggtttatgaacgcgataacgacagggaggccaggatctttggtggaacgctggacctccataagggaagcggacaagaagccatgaaaaaagcaggactgttgcagacttactatgatcttgctcttccaatgggtgttaatattgcagataaaaagggaaacattttgtctacaaaaaacgtgaaaccggagaacaggtttgacaaccctgaaattaacaggaacgatctgagagcgattcttctgaatagcctcgaaaacgacacggtcatctgggaccggaaattggtgatgctcgaacccggaaagaaaaagtggacactgacctttgagaataagccctcagaaacggcggacctcgtcatacttgctaatggcggtatgtcgaaggtccgcaaattcgtcactgacacagaggtggaggaaactgggacctttaacatccaagccgacatacaccaaccagagatcaattgccctgggttttttcaactctgcaacggaaatagacttatggcgagccatcaaggcaaccttctctttgcgaaccctaacaataacggggcactgcatttcggtatctcctttaagaccccagacgagtggaaaaaccaaacacaagttgactttcagaaccgcaactccgtggtcgattttctgttgaaagagttcagcgactgggacgaaaggtataaagagcttatacacactaccctgtctttcgtggggctcgccacacgcatctttcctctcgagaagccctggaaatctaagagacccctcccgatcacaatgatcggggatgcggcccaccttatgcccccctttgccggccagggggtgaactctggactggtcgatgccctgatactttctgacaaccttgcagacggaaaatttaattccatagaagaagctgttaagaactacgaacagcagatgttcatatatggcaaggaagctcaggaggagtctactcagaatgaaatcgagatgttcaagccagatttcactttccaacaactcctcaatgtgtaa

SEQ ID NO: 25 - DNA, 1167 bp, Artificial
  Codon Optimized for Kluyveromyces Lactis
atgacaatgaggattgatacagataagcaaatgaacctactttctgacaagaatgttgccatcattggtggtggccctgttggattaacaatggctaagttactacagcaaaatgggattgatgttagtgtgtatgaaagagataacgatcgtgaagctaggatatttggtggcactcttgatttgcacaaagggtctggccaagaggcaatgaagaaagctggcttgttacagacttattatgatcttgctttacccatgggagtcaatatcgccgataaaaagggtaacatcttatcgaccaagaatgttaagccagagaaccgttttgataatccggaaatcaataggaatgacttgagagctatattactaaactccttggaaaacgacactgtcatttgggataggaagttagttatgttggagcccggaaaaaaaaaatggacgttaacattcgaaaacaaaccatctgaaacagcagacctagttattctagcaaatggcggaatgtcgaaagtcagaaaatttgtcactgacacagaggtagaagaaacaggcacattcaacattcaggctgatatacatcaacctgagattaactgccctggttttttccaactatgtaacgggaatagacttatggcctcgcaccaaggcaatttgttgtttgctaacccgaacaacaacggggctcttcactttggaattagttttaagacgcccgatgagtggaagaatcagacgcaggtggatttccaaaataggaactcggttgtcgacttcctattaaaggagtttagtgactgggacgaaagatataaggaattgatccacacaacactatctttcgtgggcctagcaaccaggatattccctctagagaagccctggaagagtaaaagacccttaccaattacaatgatcggcgacgctgctcacttaatgccacccttcgctggccaaggggtgaactcagggttggtcgatgcattgatcttgtctgataaccttgccgatggtaagtttaactcgatcgaagaagcagtaaaaaactacgagcagcagatgtttatctacggtaaagaagcacaagaagagtcaacgcaaaacgaaattgagatgtttaaacccgatttcacatttcagcagcttcttaacgtctaa

SEQ ID NO: 26 - DNA, 1167 bp, Artificial
  Codon Optimized for Leishmania Donovani
atgacgatgcgtatcgataccgataagcagatgaacctcctttctgacaagaatgttgcgatcattggcgggggcccagtggggcttacgatggcgaagctcctccaacaaaacggcattgacgtctctgtgtacgagcgggacaacgaccgtgaagctcggatctttgggggcacgctggatctccataaagggagcgggcaagaggcgatgaaaaaggcagggcttcttcaaacctattatgatcttgcactcccgatgggtgtgaacattgccgataagaaggggaatattctctcgaccaaaaatgttaagcctgagaatcgctttgacaatccagagatcaaccggaacgaccttcgggccattctgctgaacagcctggaaaatgacactgtcatctgggaccggaaactcgtgatgcttgaacctggcaagaaaaaatggacgctgaccttcgaaaacaagccgtctgaaacggccgaccttgtgatcctggccaatgggggcatgtctaaagtccgcaagtttgtgactgacactgaagtcgaagaaactggtacttttaacatccaagcggacattcatcaaccggagattaattgtccaggttttttccaactgtgtaacggtaatcgcctcatggcatcccatcaaggtaatctcctgttcgcgaaccctaacaataacggcgctctgcatttcggcatttctttcaagacgcccgacgagtggaagaatcaaacgcaagttgattttcaaaaccgtaattccgtcgtcgatttcctgctgaaggaatttagcgactgggatgaacggtacaaggagctgattcacactacactgagctttgtcggccttgcgactcggatcttcccccttgaaaaaccgtggaagagcaaacgtccgctgccaatcacgatgattggtgacgctgcccatcttatgcccccctttgcagggcaaggcgtcaattctggtctggttgatgccctgattctgagcgataatctggcggacggcaaatttaactccattgaagaagcagtgaagaattatgagcagcagatgtttatctacgggaaagaagcacaggaggaaagcacccaaaatgagatcgagatgttcaaaccggattttactttccagcaactcctcaacgtctaa

SEQ ID NO: 27 - DNA, 1167 bp, Artificial
  Codon Optimized for Macaca Fascicularis
atgacaatgcggatagacactgacaaacaaatgaatttgcttagcgacaagaatgtagcaataattgggggtgggccagtcggtcttactatggccaaattgttgcagcaaaatgggatagacgttagtgtatatgaacgggataatgatcgcgaggcacgaatattcggcggtactctggacctgcataaaggtagtggacaagaggcaatgaagaaggcaggcctccttcaaacttattacgatttggctctgccaatgggcgtaaatattgccgataaaaagggtaacatacttagtacaaaaaatgtcaaaccagaaaaccgctttgacaatcccgagataaaccggaatgacctccgggccatccttttgaacagtcttgaaaatgacactgtaatctgggaccgaaagttggttatgttggagccaggcaagaagaagtggactctgacctttgagaataagccaagcgaaactgctgatctggtcatactggctaacgggggcatgagcaaagtacgaaaattcgttactgacacagaagtggaagagacagggacattcaatatccaagctgacatccaccagcctgaaataaactgtccagggtttttccagctctgtaatggcaacaggctgatggcaagtcaccagggaaacctgctgtttgccaatccaaacaacaatggggcacttcattttggaatctcattcaagactccagatgagtggaagaatcaaacacaggttgatttccagaatcggaatagcgttgtagatttcctccttaaagagttttccgactgggacgagcggtacaaggagctgattcacactactctgtcctttgtagggcttgctacacggatattccctcttgaaaagccctggaaatcaaagcgcccactccccatcacaatgataggagatgcagcccatcttatgcctccttttgccgggcaaggcgtcaatagtggcctcgtggacgccctgatactctcagataaccttgctgacggtaaattcaattctatcgaagaggccgttaagaactatgagcagcaaatgtttatttacggcaaggaagcccaagaggaatcaacccaaaacgagattgaaatgttcaagcccgatttcaccttccagcagttgttgaatgtctaa

SEQ ID NO: 28 - DNA, 1167 bp, Artificial
  Codon Optimized for Manduca Sexta
atgaccatgagaatagatacggacaagcaaatgaacttattgtcggataagaacgtagctattataggcggcggtccggttggcctcactatggcaaaactgcttcaacaaaacggcatcgatgtcagcgtgtatgaaagggataatgaccgcgaagcaaggatctttggcggtacacttgatttacataaaggctctggacaagaggcaatgaaaaaggcaggattgttacagacttactatgatctggctctgcccatgggagtgaatatagcggataagaagggtaatatcttatcgacgaaaaacgtgaagcccgagaaccgctttgataacccagaaatcaaccgaaatgatttaagggctatattactgaactctcttgagaatgatacagtgatatgggaccgtaaacttgtaatgcttgaacccggcaaaaaaaagtggaccctgacctttgagaataaaccgtccgaaacggcagacctcgttatcttggcaaatggaggcatgagtaaagtaaggaaatttgttacggacactgaggtcgaggaaacaggcacctttaatatccaggcagatatacaccagccagaaataaattgcccgggctttttccagctttgcaacggtaacaggcttatggcatcacatcagggtaacttactgtttgcaaatcctaacaacaacggagccctccattttggcatctcttttaagactcccgatgagtggaagaatcagacccaagtcgacttccagaatcgaaacagcgtggtagattttctcctgaaagagttttcggattgggatgaacgctataaggagctgattcatacgacgctgtccttcgtaggcttagccactcgcatcttccccttagaaaaaccgtggaagtcgaagagacccttacccatcaccatgatcggtgatgcagcccacttaatgccccccttcgccggacagggcgttaactcgggtttggtcgacgcactcatcctgtcggataatttggcagatggcaagttcaactctattgaggaagcagttaagaactatgagcaacaaatgtttatatatggcaaagaagcgcaggaagagtccacacagaacgaaatagaaatgttcaagccggactttacgtttcagcaactgctcaatgtctaa

SEQ ID NO: 29 - DNA, 1167 bp, Artificial
  Codon Optimized for Medicago Sativa
atgactatgaggatcgacaccgataaacagatgaacttgctttcagataaaaacgtcgcaattattggaggaggcccagtgggcttgactatggctaaactcttacaacagaacgggatcgatgtttctgtgtacgaaagggacaatgacagggaagcaaggatttttgggggaactcttgatttgcacaaaggttccggacaggaagccatgaaaaaggcaggattattacagacctattatgacttggccttacctatgggggtgaatatcgcagacaagaaagggaatatattgagcaccaagaatgtgaaacctgaaaataggtttgacaacccagagattaacagaaacgatttacgtgcaatcttattgaattcacttgaaaatgacaccgtaatatgggataggaagttagtaatgttagaacccgggaagaagaaatggacattaacattcgaaaataaacctagtgaaactgccgatttagttatactcgctaacggggggatgtcaaaagtccgtaaatttgtaaccgacactgaagtagaggaaactggaactttcaatattcaggccgacatacaccagccagaaattaactgcccagggttcttccaattatgcaatggaaataggctcatggcctcccatcagggcaatttacttttcgcaaatccaaataacaatggtgcactccacttcggaatctcctttaagacaccagacgagtggaagaatcaaactcaagttgatttccaaaaccgtaacagcgtggtggattttttattgaaggagttcagcgactgggatgagaggtacaaagagttaatccacaccaccttgtctttcgtcggcttagctaccagaatcttcccactcgaaaaaccctggaagtcaaagaggccattacccattactatgattggtgacgctgctcacctcatgcctcccttcgctgggcagggagtaaactcaggcttagtagacgctctcattttgtccgataacttggctgacggcaaatttaactctatcgaagaggccgtaaaaaactacgaacaacaaatgttcatttatggcaaagaagcacaggaagaatctacccaaaatgagatagaaatgttcaagccagattttaccttccagcagttgttgaacgtctaa

SEQ ID NO: 30 - DNA, 1167 bp, Artificial
  Codon Optimized for Neurospora Crassa
atgaccatgcggattgataccgataagcaaatgaaccttttgagtgataaaaacgtggccattattggtggcggaccggttggactgactatggcaaaattgctccagcaaaacggcattgacgtgtctgtctatgaaagggacaacgaccgggaagcgcgtatctttggaggcacactggatctgcacaagggaagcggtcaggaagcaatgaaaaaggcagggctcctccagacttattacgatttggccctccctatgggtgtgaacattgctgacaaaaaggggaacatcctgagcactaaaaatgtcaagcccgaaaatcggttcgacaacccggagatcaaccgaaacgacctccgagcaattctgctgaattccctcgaaaacgatacagtcatctgggaccgaaagctcgttatgctggaacccgggaagaaaaagtggacacttaccttcgagaacaaaccctcggaaaccgccgatcttgtgatcttggctaacggcgggatgtcgaaagtgcgtaaattcgttacggacacagaagtggaagagactgggacattcaatatccaagctgatattcaccaacctgaaatcaattgtcccgggtttttccagttgtgtaatggtaaccgacttatggcttcccaccagggaaatctgctcttcgcaaatcccaacaataacggggcacttcacttcggcatttcttttaaaactcccgacgagtggaaaaatcagactcaggtcgacttccagaatcggaattcagtcgtggacttccttttgaaagagtttagtgattgggatgaaaggtacaaggaactcattcacacaaccttgtctttcgttggacttgcgactcggatttttccgttggagaagccctggaagtcaaagagaccgttgccgattaccatgatcggcgacgcagcgcacctgatgccgccatttgctggacaaggtgtcaattccggcttggttgatgctttgatcctgtctgacaatcttgcggacggtaagttcaattccattgaggaggctgttaaaaactatgagcagcagatgttcatttatggaaaagaagctcaagaagagtccactcaaaatgaaatcgaaatgttcaaaccagactttacttttcagcagttgcttaacgtctaa

SEQ ID NO: 31 - DNA, 1167 bp, Artificial
  Codon Optimized for Nicotiana Benthamiana
atgacaatgcgtatagataccgataagcagatgaatcttctttcagataagaatgttgcaataattggcggtggacccgtaggactcacgatggctaaactcctgcagcaaaatggaatagatgtaagcgtatacgaacgtgacaacgaccgtgaagcaaggatatttggcggtactcttgacttgcataaggggagcggccaagaagcaatgaaaaaagctggcctgcttcagacctattatgacctcgccttgcccatgggagtaaacattgcagataagaagggcaacatcctgtccaccaagaacgttaagccggaaaatcgattcgataacccagaaataaaccgtaacgatttgagagcaatcttattgaatagtttagagaacgataccgttatatgggataggaaactcgtgatgttggaaccgggaaaaaaaaagtggacccttacgtttgagaacaagccatctgagaccgctgacttagtaatcctcgcaaatggagggatgagcaaagtaagaaaattcgtcactgacacagaagtggaagagacaggtacatttaatatacaagccgatattcatcagcccgaaatcaactgccccggattttttcaattatgtaacggaaaccgacttatggccagtcatcaaggtaaccttctttttgctaatcctaacaacaacggggcattgcacttcgggatcagctttaaaacacccgatgagtggaagaatcagacgcaagtagacttccaaaatcgaaattccgtggtagattttctgttaaaagagttttcagattgggatgagcgatataaagagcttattcatactacgctttctttcgttggtctggccaccagaatctttcccttggagaagccgtggaagtctaaaagacccttacccattactatgataggtgacgctgcacatcttatgcctccttttgccggtcagggcgttaactcaggtttagtcgatgctctcattcttagtgacaatttagccgacgggaaatttaactcaattgaggaagctgtaaagaactacgaacagcaaatgtttatctatggtaaagaggctcaggaggaaagtacgcagaatgagattgaaatgttcaagcctgacttcacgttccaacagttattaaacgtataa

SEQ ID NO: 32 - DNA, 1167 bp, Artificial
  Codon optimized for Nicotiana tabacum
atgactatgaggattgatactgataagcagatgaacttgttgagtgataaaaatgttgctataattggtggtggacctgttggattgactatggctaagcttttgcaacaaaatggtattgatgtttctgtttacgaaagagataatgatagagaggctaggatttttggaggtactcttgatttgcataagggatcaggtcaagaagctatgaagaaagctggtcttttgcaaacatactacgatcttgctttgccaatgggagttaacattgctgataagaagggtaacattttgtctacaaagaacgttaagccagaaaacagattcgataaccctgagattaatagaaacgatttgagggctattcttttgaactcacttgaaaacgatactgttatttgggataggaaacttgttatgttggagccaggaaagaaaaagtggactcttacattcgaaaataagccttctgagacagctgatcttgttattttggctaatggtggaatgtcaaaagttagaaagtttgttactgatacagaagttgaagagactggaacttttaatattcaagctgatattcatcaaccagagattaattgtcctggatttttccaattgtgtaatggtaataggcttatggcttctcatcaaggtaatcttttgttcgctaacccaaataacaatggtgctcttcattttggtatttcttttaagactcctgatgagtggaagaatcaaacacaagttgatttccaaaacagaaactctgttgttgattttcttttgaaagagttttcagattgggatgaaaggtataaggagcttattcatactacattgtcttttgttggacttgctactagaatttttccattggaaaaaccttggaaatcaaagaggccacttcctattacaatgattggagatgctgctcatcttatgccaccttttgctggacaaggtgttaattctggattggttgatgctcttattttgtcagataatcttgctgatggaaagtttaattctattgaagaggctgttaagaactacgaacaacaaatgttcatgtatggaaaggaggctcaagaagagtcaactcagaatgagatagagatgttcaagccagattttacttttcagcagttacttaatgtgtag

SEQ ID NO: 33 - DNA, 1167 bp, Artificial
  Codon Optimized for Oncorhynchus Mykiss
atgacaatgcggatagacacggataagcaaatgaatctcctcagcgataagaacgtggctatcattggcgggggtcctgtcggcctcaccatggcaaagctgctgcaacagaatgggattgatgtctccgtgtatgagcgggataatgaccgggaggccagaatattcggaggcaccctggacctccacaagggaagcggccaagaggccatgaaaaaagctggtctcctgcaaacgtattatgatctggctctgcccatgggcgtcaacattgccgataaaaaggggaacatactcagcacgaagaatgtcaaacccgagaatcgctttgataaccccgagatcaatcggaatgatctgcgcgccatcctgctcaactccctggagaacgatactgttatttgggaccgcaagctcgttatgctggaacctggcaagaaaaagtggactctcacttttgaaaataaaccctctgaaaccgccgacctggtgatcctggctaatggaggtatgtctaaagttcgtaagtttgtcacggatactgaagtggaagagacggggacattcaacatccaagctgatatacaccagcccgagataaattgccccggattcttccaactgtgcaatggcaatcgtctgatggcttcccaccaaggaaatctgctgttcgctaaccccaataacaacggtgctctgcattttggtataagtttcaaaacgcctgacgaatggaaaaaccagacccaggtggatttccaaaaccgtaactcagttgtggactttctcctgaaagaattcagtgattgggatgagaggtacaaggagctcatacatacgacgctgtcatttgtcggactggctacgaggatttttccactcgagaagccatggaaatctaagcgccctctgccaattacaatgattggtgatgctgcccatctgatgcccccatttgcagggcagggtgtcaacagtgggctggttgacgctctcattctgagtgacaatctcgcagatggtaaatttaatagcatagaagaggctgtgaaaaattacgaacagcagatgtttatatacggcaaggaggcacaggaggagtcaacccagaatgaaatcgagatgtttaagcccgactttacgttccaacaactcctcaacgtctaa

SEQ ID NO: 34 - DNA, 1167 bp, Artificial
  Codon Optimized for Oryctolagus Cuniculus
atgactatgcggatagacacagataagcagatgaatctcctgagcgacaagaacgtcgctattattggcggtggccccgtcggtctcacaatggctaaactcctccagcagaatggaatagatgtctccgtttacgagcgcgacaatgatcgcgaagccagaatctttgggggtacattggatctccacaaaggcagtggtcaggaagccatgaagaaggcaggtctgctgcagacgtattacgatctggcgctcccaatgggagtgaatatagctgacaaaaaaggcaacatcttgagcactaagaatgttaaaccggagaaccgcttcgacaaccccgaaattaatcggaacgatctcagggccatactgctgaactcactcgaaaatgataccgtcatttgggaccgcaagctggtcatgctcgaacccggcaaaaagaaatggacgctcacgtttgagaataagccctctgaaactgcggatctcgtcatcctggctaatggaggaatgagcaaggtgagaaaatttgttacggacacggaggtggaagagacgggaacatttaatatacaagcagacattcatcagcccgaaataaactgccccggattcttccaactctgcaatggtaacaggctgatggcgtctcatcaggggaatctgctgtttgcaaaccctaataataacggtgcactgcacttcggaatctcttttaaaacccccgacgaatggaagaaccaaacgcaagtggactttcagaaccgcaactccgttgtggatttcttgctgaaagagttctctgattgggatgagagatataaggagttgatccacacaaccttgtcatttgtgggattggcaactagaatctttcctttggaaaagccctggaagtctaagaggccactcccgataacaatgatcggtgacgccgcccatctgatgcccccctttgccgggcaaggtgttaattcaggtctggttgacgccttgatcctcagcgacaacctcgcggacgggaaatttaattcaatcgaagaagcagttaagaactacgaacagcagatgtttatttatggcaaggaagcgcaagaggaatctacacaaaatgaaattgaaatgttcaagcccgactttaccttccagcagctgctgaatgtgtaa

SEQ ID NO: 35 - DNA, 1167 bp, Artificial
  Codon Optimized for Oryza Sativa
atgaccatgaggatagacaccgacaaacagatgaatcttctcagtgacaagaatgtggcgattattggcggcgggccggtcggattgactatggctaagctccttcagcagaacggtatagatgttagcgtctacgaacgcgataatgatagagaagcgaggatttttggcggcaccttggatcttcacaaaggttccggccaagaagctatgaaaaaagctggacttctgcaaacttattatgatcttgcgttgcctatgggtgtcaatattgcggacaagaaagggaatattttgagcacgaaaaacgtcaaaccggagaatagatttgataacccggagataaacagaaatgatctccgggccatattgctcaactctctggaaaacgacactgttatatgggacaggaagctggtcatgcttgagcccggcaaaaagaaatggactctgacttttgaaaataagcctagcgagacggcagatctcgtcatattggctaacgggggaatgtctaaggtgagaaagtttgtgactgacaccgaagtggaagaaacaggcaccttcaatattcaggccgatatacaccaacccgagattaactgtcctggattctttcaactgtgtaatggaaaccggctcatggcgagtcaccaggggaatcttctctttgccaatccaaacaacaatggggccctccactttggcatatcatttaagacgcctgacgaatggaagaatcaaacacaagtcgattttcaaaatcggaattcggtggtcgacttcctcctgaaggaattctccgactgggacgagaggtacaaagagctgattcataccactctttcctttgtgggtctggctacgcgcatcttcccgctcgaaaaaccatggaaatctaaacggccattgccaatcaccatgataggagacgcggcacaccttatgccgccattcgccggccaaggggtgaacagtggcctcgttgacgctctcatactgtcagacaacctggcggatggaaagtttaactcgatagaggaggcggttaagaactatgagcagcaaatgttcatttacggcaaggaggctcaggaggaatctacgcagaatgaaatagagatgtttaagccagatttcacgttccaacagttgctgaacgtctaa

SEQ ID NO: 36 - DNA, 1167 bp, Artificial
  Codon Optimized for Ovis Aries
atgacaatgcgtatagacacagataaacaaatgaatcttctgagtgacaaaaatgtcgccattattgggggtgggcccgtcggtctgacaatggcaaaactgctccagcaaaatggtatagatgtttctgtttacgaaagagacaatgacagggaagccagaatctttggggggacactcgacctgcacaaggggtccggacaagaagccatgaagaaagctggccttctccaaacttattacgaccttgccctccctatgggagttaatatcgcagataagaaaggtaatatacttagtactaaaaatgtcaagcctgagaatcgcttcgacaatcccgaaatcaatagaaatgatcttcgcgccatactgctgaattccctcgagaacgatacagttatatgggatagaaaactcgtcatgctcgagcccggaaagaagaagtggacactcaccttcgagaacaagccatccgagactgctgatctcgtcattctcgcaaatggagggatgtccaaggtgcggaagtttgtgacagatacagaagtcgaggaaacaggtacatttaacattcaagccgacatacatcagcccgaaattaactgcccaggattctttcaactttgcaatggaaatcgtctgatggcatctcaccaagggaacctgctgttcgccaatcccaacaacaacggagctctgcattttggaataagtttcaagacaccagacgaatggaaaaatcaaacccaagtcgactttcagaatcggaacagtgtggtggactttctccttaaagaattctccgattgggacgaacgttataaggagcttatccacaccaccctcagctttgttggcctcgccactcgtatattccctcttgaaaaaccttggaagtccaagagacccctcccaataactatgataggagacgccgcacacctgatgccaccattcgctggacagggcgttaattctggacttgtcgacgccctgatactttctgataacctcgcagatggtaagttcaactccattgaggaggcagtgaagaactacgaacagcagatgtttatatacggtaaagaagctcaggaagagagcacccagaatgagatcgagatgttcaaacctgacttcacttttcagcaactgctcaatgtgtaa

SEQ ID NO: 37 - DNA, 1167 bp, Artificial
  Codon Optimized for Petunia x Hybrida
atgactatgcgaatcgatacagacaaacaaatgaatttgttatccgacaagaatgtcgccattatcggtggcggtccagtcggactaactatggcaaaactactacagcagaacggcattgacgtatccgtgtatgaacgtgacaacgatagagaagctcgtatttttggcggcaccctcgatttgcacaaaggcagtggtcaagaggctatgaaaaaggccggccttcttcaaacctactatgacttggcacttccaatgggagtcaacatcgctgataagaaagggaatattttgagtaccaagaatgtaaaacctgagaatcgttttgataaccccgagattaacagaaacgacctcagggctatccttcttaacagtctagaaaatgataccgtcatatgggataggaagctcgtgatgctcgagcctggtaagaagaagtggaccctcacattcgagaataaaccttctgagaccgctgacttggtgatcttggctaatggcggaatgagcaaagttaggaaattcgttacagatacagaagtagaagagacaggaacttttaatatacaagccgacattcatcaaccagagattaactgccctggcttcttccagctctgtaacggcaaccgtttaatggcctctcatcagggtaatcttttgtttgcaaatcccaataacaacggagcattacattttgggatctccttcaaaacccctgacgaatggaaaaaccaaacacaagtggattttcaaaaccgaaactctgtggtggatttcctcttaaaagaattctcagattgggatgaaaggtataaggagttaatccacacaacattaagttttgtcggccttgctacaaggattttccctcttgagaagccctggaaatctaaaaggcccctacctattaccatgatcggagatgcagctcaccttatgccaccattcgcaggtcagggggttaacagtgggttggtcgatgctttgattctaagcgataacctcgccgatggaaaatttaattccatagaagaggctgtaaagaattatgaacagcaaatgttcatttatgggaaggaggcccaagaagagtcaactcagaacgaaattgagatgttcaagcctgattttactttccaacagctactcaatgtataa

SEQ ID NO: 38 - DNA, 1167 bp, Artificial
  Codon Optimized for Phaseolus Lunatus
atgacaatgcgcatcgataccgataaacaaatgaacctcctgtctgacaagaatgtagcaataattgggggtgggcccgtagggcttactatggccaaacttttgcagcaaaatggcatagatgtgtccgtgtacgagagagataatgaccgtgaagcccgtatcttcggcggaacgttggatcttcacaaagggtctgggcaggaagctatgaagaaagctggactcctccagacttactacgatctggcactccctatgggggtcaacattgccgacaagaagggaaacatactcagcaccaagaatgtcaagcctgaaaataggtttgacaaccccgagatcaataggaacgacctcagagccattttgcttaatagtctcgagaacgatacggtaatctgggatcgtaagctcgtcatgttggaacccggtaagaagaaatggactctgacctttgagaataagccatcagagactgcagacctcgtgatcctggctaacggaggtatgagcaaagtgcgcaaatttgtgaccgacacagaggttgaggaaaccggaacctttaatatacaagccgatattcatcagcctgaaatcaactgtccaggattctttcaattgtgtaacggcaacaggctgatggcatcacatcagggtaatctgctcttcgcaaaccctaataacaatggggcacttcactttggaatttctttcaaaacgcccgatgaatggaaaaatcagacgcaggtggacttccagaatagaaactctgtcgttgactttcttcttaaggaatttagcgattgggatgagcgctataaggagctgattcatactactttgagttttgtgggtcttgctacgcgtatattccctcttgaaaagccctggaagagcaagcgccccttgccaataacgatgatcggcgatgccgcccatctgatgcccccttttgccgggcagggggtgaacagcggtctcgttgatgctctcattctctcagataacttggctgatggcaagtttaactccattgaagaagcagtaaagaactacgaacagcaaatgtttatatatggcaaggaggcacaagaggagtctacgcagaatgagattgaaatgtttaaacccgacttcacattccaacaactgctgaacgtataa

SEQ ID NO: 39 - DNA, 1167 bp, Artificial
  Codon Optimized for Pisum Sativum
atgactatgagaatcgacaccgataaacaaatgaacctccttagtgataagaacgtagcaattattgggggcggtccagtgggacttaccatggcaaaattgttacaacagaacggaatcgacgtcagtgtttacgagcgtgataacgatagagaagccagaatattcggaggcactttggatttgcataaagggtctggtcaagaagcaatgaaaaaggctggtttgttacagacctattatgatctcgccctccctatgggggtgaacatagctgacaaaaagggtaatatcctttccaccaagaacgtgaaacccgagaaccgttttgataacccggagataaaccgtaatgatttaagggcaatattgttgaattccctcgagaacgacactgtcatttgggatcgtaagcttgttatgttagagcctgggaagaagaagtggacattgacttttgaaaataagccatctgagaccgccgatctcgttatcctcgctaacggaggcatgtcaaaggttaggaaattcgtcacagataccgaggtggaagaaaccggtactttcaacatacaggcagatatccaccaaccggagattaattgccccggattttttcaattatgcaatgggaacaggctcatggcttctcatcagggcaacttattatttgccaacccgaacaacaacggcgccttacattttggaattagcttcaagacacctgatgaatggaaaaaccagacacaagtagactttcaaaacagaaactcagtggtagattttcttttaaaagaattctctgattgggatgaaaggtataaggaattgatccatactacactttcattcgtagggttggccaccagaatcttcccacttgaaaagccttggaaaagcaagcgtccacttcctatcactatgattggtgacgctgcccatcttatgcctcccttcgctggccagggagtgaactcagggttggttgacgctttaatactctccgacaaccttgctgatggaaagttcaactcaatcgaggaggccgttaagaattacgaacaacaaatgttcatttacgggaaagaagcacaggaggaatcaacccaaaatgagattgagatgtttaagcccgattttacattccagcagctcttaaatgtctaa

SEQ ID NO: 40 - DNA, 1167 bp, Artificial
  Codon Optimized for Plasmodium Falciparum 3D7
atgaccatgcgtattgatactgacaagcagatgaacttgttgtctgataagaatgttgccataattggtggtggacctgtaggtttgactatggctaaattacttcagcagaacggaattgacgtgtcagtttacgagagggataacgatcgtgaagctcgtatattcggaggaactcttgatcttcataaaggatcaggtcaggaggcaatgaagaaagccggattgttgcaaacatattacgacttagctcttccaatgggagttaacatagctgataaaaagggaaacatattatcaacaaagaacgtaaagcctgaaaacaggttcgataacccagaaataaataggaacgatcttagggctattcttcttaactctttggaaaatgacactgtaatatgggacagaaagcttgtgatgttagagcctggtaagaaaaagtggacattgacctttgaaaacaaaccatctgagaccgcagacttagttatacttgctaacggtggaatgtcaaaggttaggaagttcgtaacagacactgaagtagaagaaacaggaacctttaacattcaagcagatattcaccagccagagattaattgccctggttttttccaattgtgcaatggaaacaggcttatggcatcacatcaaggaaacttgttattcgccaacccaaacaacaatggtgccttgcacttcggtataagtttcaaaacccctgatgaatggaagaaccagacacaggtagactttcagaatcgtaattctgtagttgacttcttattgaaggagttttctgattgggacgaaagatacaaagagttgatacataccaccctttcattcgtaggacttgccactcgtatatttccattggagaaaccttggaaatctaagcgtcctcttcctattactatgataggagatgcagctcaccttatgccaccattcgctggtcaaggagtaaactctggacttgtggacgcacttattttgagtgacaatcttgccgatggtaaattcaattcaatagaggaggctgtgaaaaattatgagcaacaaatgtttatttatggtaaggaggctcaagaggagtctactcagaacgaaattgaaatgttcaagccagacttcacattccaacagcttttaaacgtgtaa

SEQ ID NO: 41 - DNA, 1167 bp, Artificial
  Codon Optimized for Rattus Norvegicus
atgaccatgcgcattgacacggacaagcaaatgaaccttttgtctgataagaatgttgctattatcggagggggacccgtggggctgacaatggccaagctgctgcaacaaaatggaatagatgtcagtgtatacgagcgggacaatgacagagaggcccggattttcgggggtacactcgacctgcacaaaggatcaggtcaagaggcaatgaagaaagcagggctgttgcagacatactatgatcttgcacttcctatgggtgttaatatcgccgataaaaagggtaacatactctccaccaaaaatgtgaagcccgagaaccgcttcgataaccctgagattaaccggaacgaccttcgcgcaatcttgcttaactcattggagaatgacacggtaatctgggacaggaaactcgttatgcttgagcccggaaaaaaaaagtggaccttgacttttgaaaataagccttccgagacagctgatcttgtaatcttggccaacggcggaatgtccaaagtccgaaaatttgtaaccgatacagaggtggaggagacaggaacattcaatatacaagctgacatccaccaacctgaaattaattgccctggctttttccaactgtgtaatgggaacaggctcatggcctctcatcaaggtaaccttcttttcgctaacccgaataacaatggcgcccttcatttcggtatcagcttcaaaacgcccgatgaatggaaaaatcagacccaggttgactttcagaaccgaaattcagttgttgattttctgcttaaagagttctccgactgggatgagagatataaagagcttatacacacgacattgagttttgtaggcttggcaacaagaattttccctttggagaaaccctggaagagtaagagacccctgcctattactatgatcggtgatgctgcacatctcatgccccccttcgctgggcaaggtgttaattcaggtcttgttgacgccctgatattgagtgataacttggcagatggaaaatttaattctatagaggaagccgtcaaaaattatgaacagcaaatgtttatctacggaaaagaggcccaagaagaaagcacccagaatgagattgaaatgtttaaacctgacttcacattccaacagctgctcaacgtataa

SEQ ID NO: 42 - DNA, 1167 bp, Artificial
  Codon Optimized for Salmo Salar
atgacaatgaggattgacactgataagcagatgaatctgctgtccgacaaaaacgtcgcaataataggtggtggtccagtcggattgaccatggccaagctcttgcaacagaacgggatagatgtctcagtctatgaaagagataacgatagagaagcccgcatcttcggggggaccttggatctccacaagggttccgggcaagaagctatgaagaaagcagggctcttgcagacctattacgatctggctctccctatgggtgtgaacatcgcggacaaaaaaggaaacattctctctacaaaaaacgttaaacccgaaaatcggtttgacaaccctgagataaataggaatgatctccgcgcaattctgctcaacagcctggaaaacgacacagtcatttgggaccggaagttggtcatgctcgagcctggtaaaaagaaatggacgctgacatttgagaacaaaccctccgagactgcggatttggtgattctggcgaatggggggatgagtaaggtcaggaaatttgtgaccgatactgaggtcgaagagactggtactttcaacatccaagccgacattcatcaaccggaaataaattgtcctggctttttccagttgtgcaacggtaaccgtctcatggcatcccatcagggcaacctgctctttgctaatccgaacaataacggagcgctgcacttcggcatatctttcaaaaccccggacgaatggaagaatcagacccaggttgattttcaaaacagaaattcagtcgttgatttcctgctgaaggagttttctgattgggatgaacggtataaagagttgatacatacaaccctctcatttgtgggtctggctacgcggatcttcccgctcgaaaaaccgtggaaatcaaagcgcccgctgcctattacgatgataggcgatgccgctcacctcatgcctccctttgctggtcagggggttaattctgggctggtcgatgcactcatcctcagcgataatttggctgatggtaagttcaattccatcgaagaagcggttaagaattatgagcagcaaatgtttatctatgggaaggaggcccaggaggaaagtacgcaaaatgagatagagatgtttaagccagattttacttttcagcaactcctgaatgtctaa

SEQ ID NO: 43 - DNA, 1167 bp, Artificial
  Codon Optimized for Schistosoma Mansori
atgacaatgcgaatcgatacagataagcaaatgaatctattgtctgataaaaacgtagccatcatcggcggaggtcctgtaggtttaacaatggccaaattgttacaacaaaatggtatagacgtttcagtttacgagagagataacgacagagaagctcgaatttttggcggcactttagacctacataaaggcagtggtcaagaggctatgaaaaaggctggtttgcttcagacttattatgatcttgcactaccaatgggtgtgaacatagcagacaaaaagggaaacattttatctactaaaaatgtgaagccagagaatcgattcgacaatcctgaaatcaatagaaacgatttgcgtgccatcttattgaattctcttgaaaacgacacagtaatttgggaccgaaagttggtaatgttggagcctggcaaaaagaaatggacattaactttcgagaataaaccttctgagactgccgatttggtcatcttggccaatggcggcatgtctaaagtccgaaagtttgtcacagacacagaagtagaagaaactggaacttttaatatacaagctgatatacaccagcctgaaattaattgtcctggtttcttccagctatgcaacggcaaccgtcttatggccagtcaccaaggcaatcttctttttgcaaatccaaataataatggcgcattgcattttggcatatcatttaagacacctgacgaatggaagaatcagactcaggtggattttcaaaatcgaaattcagtggtagattttcttttgaaggagttcagtgattgggatgagcgttacaaggaacttatccatactacattgtctttcgtgggccttgccacacgtatctttccattagagaaaccatggaagagtaagcgtcctttaccaatcactatgataggcgacgctgctcacttaatgcctccttttgccggtcaaggcgtcaacagtggattagttgacgctctaattttatcagataaccttgcagacggcaagtttaatagtatcgaagaggccgtcaaaaactatgagcaacaaatgttcatatacggaaaggaagcccaagaggaaagtactcaaaacgaaatcgaaatgttcaaacctgatttcactttccaacaacttcttaacgtttaa

SEQ ID NO: 44 - DNA, 1167 bp, Artificial
  Codon Optimized for Schizosaccharomyces Pombe
atgacaatgcgcattgatactgataaacagatgaaccttcttagtgacaagaatgtggctataataggcggcggaccagttggccttactatggcaaaattattacaacagaacggtatagatgtatctgtttatgaaagagataatgatcgtgaagcacgaatttttggaggtacattggacttacacaaaggatccggtcaagaagcaatgaaaaaggctggcttattacagacctattatgacttggcattacctatgggagttaatatagccgataaaaagggtaacattctttcaacgaagaacgtcaagcccgaaaatcgcttcgacaacccagaaataaatcgaaatgacttacgtgcaatattattgaactcccttgagaacgacacggttatatgggatcgcaaattggtaatgttggagcctggtaagaaaaaatggaccttgacttttgaaaataaaccaagtgagacggccgaccttgtaattttggccaatggaggaatgtcaaaggtccgaaaattcgtaactgatacagaggtcgaggagaccggcacgtttaatatccaagctgatatacatcaaccagagattaattgtccaggtttttttcaactttgcaacggaaatagattgatggcttctcaccaaggcaatttgttgtttgccaatcctaacaataatggtgcacttcattttggcatttcattcaagactcctgatgaatggaagaaccagacccaagtggattttcagaacagaaactcagtcgttgacttccttttaaaggagttttcagactgggatgagcgttacaaggagttgatccatactaccttgtctttcgtcggccttgcaactcgcattttccccttagagaaaccctggaaatccaaacgtcccttgcctattactatgattggtgacgccgcccacttgatgcccccattcgccggacagggagtgaattcaggcttggttgacgctttgatactttccgataacttagccgatggcaaatttaattcaatagaggaagcagtaaaaaactacgagcaacaaatgtttatatacggaaaagaagcccaagaggaaagtacccaaaacgagatagagatgtttaaaccagatttcacattccaacagttacttaacgtgtaa

SEQ ID NO: 45 - DNA, 1167 bp, Artificial
  Codon Optimized for Schmidtea Mediterranea
atgaccatgagaattgacaccgataagcagatgaatcttttgtcagacaaaaacgtggctattatcgggggcggacctgtgggtcttaccatggcaaaattacttcaacaaaacggaattgacgtgtcagtttatgagcgtgacaacgatagagaggcacgaatttttggagggacccttgacttgcataagggaagtgggcaggaggctatgaaaaaggccggtttacttcagacatattacgatttggcattgcccatgggggtgaacattgctgacaaaaaaggaaacattttatcaaccaaaaatgtgaagcccgaaaacagatttgacaatcctgagattaaccgtaacgatttaagagctatccttttaaattctttggagaatgatactgtcatttgggatagaaagttggtcatgttggagccggggaagaagaaatggactttaacctttgagaacaagccttcagagactgctgacttagtgatattagccaatggaggcatgagtaaagtacgaaagtttgttaccgatactgaagtagaagaaactggaacttttaacattcaggccgatatacatcaacccgaaatcaattgtccaggtttcttccagctttgcaatggtaatagattgatggcttcacaccagggaaatttattatttgcaaaccctaataacaatggcgcattacattttggtatctcgtttaaaacacctgatgaatggaagaaccaaacacaagttgattttcagaaccgtaattccgtggtagactttcttttgaaggaattctcggattgggatgaacgatacaaggagcttatccataccaccctttcattcgtgggattagctactagaatctttccacttgaaaagccgtggaagtcaaagcgtccattgccaatcaccatgattggggacgctgcacatttgatgccaccattcgcaggccagggtgttaactcgggacttgtagatgctttgattttatccgacaacttggcagatggaaagtttaacagtatcgaggaagctgtgaagaattacgaacaacaaatgtttatctatgggaaagaagcccaggaggaaagtacacagaacgaaatcgaaatgttcaagccggacttcacattccagcagttgcttaatgtttaa

SEQ ID NO: 46 - DNA, 1167 bp, Artificial
  Codon Optimized for Solanum Lycopersicum
atgacaatgcgtattgacacagacaagcaaatgaacctattgtcagataaaaatgttgctatcataggcgggggacctgtaggcttgaccatggcaaagctgttacagcaaaatggaatcgacgtaagtgtatatgaacgagacaacgaccgagaggccaggatatttgggggaacacttgacttacataagggatctggacaggaggcaatgaaaaaagcagggctcttacaaacttactatgacctggcacttccaatgggagtcaatattgctgacaagaagggaaatatcttatcaactaagaatgtgaagcccgaaaatcgatttgacaatcccgagataaatcgtaacgacttacgtgctattctactcaacagtttagagaatgataccgtcatatgggataggaagctagtgatgctcgaaccaggaaagaaaaagtggactctcactttcgaaaacaaaccctcagagaccgctgaccttgtgatactggctaatggaggtatgagcaaagtaaggaaattcgtaacagacacagaggtggaggagactggtacctttaatatccaagccgacattcatcaacccgagattaactgtccaggctttttccaactttgcaacggtaatcgtcttatggcatcccatcaaggcaacttgctattcgccaaccccaacaataacggggcccttcattttggtattagttttaaaacccctgatgagtggaaaaatcagactcaggtagatttccagaacaggaatagcgttgtggacttcctactaaaggagttttcagattgggatgaacgatataaagaacttattcacacaacactttcttttgtggggcttgcaacccgtatatttccacttgagaaaccatggaagtcaaaaagacccttgccaatcactatgatcggagatgctgctcacctaatgccaccttttgcagggcagggtgtgaattctgggctggtggacgctctaatcttatctgacaatcttgctgacgggaagttcaacagtatcgaagaggctgttaaaaattacgaacagcagatgtttatatatggcaaagaagcccaggaagagtccacacaaaatgaaattgagatgtttaagccagacttcactttccagcaactccttaacgtttaa

SEQ ID NO: 47 - DNA, 1167 bp, Artificial
  Codon Optimized for Solanum Tuberosum
atgaccatgaggatcgacaccgataaacaaatgaacctccttagtgacaagaatgttgccataattgggggcggacctgtaggcctaactatggcaaagctcttacaacagaacggcatagacgtaagtgtttatgaaagggacaacgatcgtgaagcccgaattttcggagggactcttgatttgcataaagggtccggccaggaagctatgaaaaaggctggacttctccagacatactacgatctagccctccctatgggagtgaacattgcagacaaaaaaggcaatattttgtcaaccaagaatgtgaaacccgagaaccgatttgataatcctgagattaataggaacgacctcagagcaatcctcctaaacagcctagagaacgacacagtcatctgggatcgaaagctagtcatgctagaaccaggcaagaagaagtggacacttacctttgagaataagcccagtgaaactgctgaccttgtgatcttagcaaacggcgggatgtctaaagttaggaagtttgttaccgacacagaggtcgaagaaacaggtacttttaacatccaagccgacatccatcagcctgagattaactgtccaggcttttttcaactatgtaacgggaaccgtctcatggcaagtcatcaggggaatttgctcttcgctaaccctaacaacaatggtgctttgcactttggaattagcttcaagacaccagacgaatggaaaaaccaaacccaagtagactttcagaataggaactccgtggtagactttttgcttaaagagttcagcgattgggatgaaaggtataaagagttgatccataccaccctatctttcgttggcctcgcaacacgtatcttccccctcgagaagccttggaaaagcaaacgtcccttgcccatcacaatgatcggcgatgctgcacacctcatgcctcccttcgctggtcaaggcgtcaattccgggctcgtcgatgcattaatcttgtccgataacctcgccgacggaaaatttaactccattgaagaagccgtcaaaaattacgagcagcagatgtttatttatggcaaagaagcccaagaagagtctacccagaatgagattgaaatgttcaagccagatttcaccttccagcaactcctaaacgtataa

SEQ ID NO: 48 - DNA, 1167 bp, Artificial
  Codon Optimized for Sorghum Bicolor
atgacaatgcgcatagataccgacaagcagatgaatctgttgtccgacaagaacgttgctattattggtggggggccggtgggcctcacgatggcaaagcttttgcaacagaatgggattgatgtctcagtttacgagcgggataacgatcgggaggcgcgtattttcggcggtactcttgatttgcataagggatcggggcaagaagcgatgaagaaagctggattgcttcaaacctactatgacttggctctgcccatgggcgtgaatattgcagacaaaaaaggaaatattctttctactaaaaatgttaaacccgaaaacaggtttgataacccagagataaaccgtaatgatctcagagctattctgttgaactccttggagaatgacacagtgatatgggacagaaagttggtcatgcttgagcctggcaagaaaaagtggactttgacgtttgaaaataagccgtctgaaaccgcagatttggtgatcctcgccaacggtggaatgtcaaaagtgcgtaagttcgtcacagacactgaggttgaagaaacagggacatttaacattcaggctgatattcaccaacccgaaataaactgccctggtttcttccagctgtgtaatggaaataggctgatggcatcccaccagggaaatttgctgttcgccaacccaaacaataacggtgcgctccacttcggcatttctttcaaaaccccagacgagtggaaaaaccagacgcaagtggactttcaaaaccggaactctgtggtggactttttgctgaaggagttttcagactgggacgaacgttataaagagctgatccacacaaccctcagctttgtgggattggctacccggatatttccgctggagaagccgtggaaaagcaagcggcctctccctattacaatgatcggagacgccgcccatttgatgcctccattcgcgggtcagggggttaattctggtcttgtggacgcactgatcttgtccgataacctcgccgatggtaaattcaacagcatagaagaggctgtcaaaaactatgagcagcaaatgtttatttacggcaaagaagcacaagaagaaagcactcagaatgagatcgagatgttcaagccggattttacgtttcagcaactgttgaatgtgtaa

SEQ ID NO: 49 - DNA, 1167 bp, Artificial
  Codon Optimized for Spinacia Oleracea
atgaccatgcgtatcgacacagataaacaaatgaaccttttgagtgacaaaaatgtggctattattggtggtggcccagttggcttaaccatggccaagttgcttcagcagaatgggattgatgttagtgtatatgagagggacaatgaccgtgaggcccgtattttcggcgggaccttagacctccataaagggtccggacaggaagctatgaagaaagctggccttctccaaacctattacgatttagccttacccatgggggtgaatatcgctgataaaaaagggaacatattgagtactaaaaacgtaaagcccgaaaacagatttgataaccccgagataaatcgtaatgatttaagggccatcttactcaacagtcttgagaatgataccgtaatttgggatcgtaaattagttatgctcgaacctggtaaaaagaaatggacactcacattcgaaaataagccaagtgaaacagctgatttagtaatattggctaacggaggcatgtctaaagtaaggaaatttgtgacagacactgaggtggaagaaaccggtacattcaacatccaagcagatattcatcagccagaaataaactgcccaggctttttccagttatgcaacggtaatagactcatggctagtcatcaaggtaatctccttttcgctaatccaaataacaatggcgccttacacttcggaattagttttaaaacccctgacgaatggaagaaccagactcaggtcgattttcaaaatagaaactctgtcgtagatttcttgctcaaagaattttcagactgggatgaacgttacaaggaattaattcacaccacattaagtttcgtggggctcgcaacaaggatatttcctctcgagaaaccctggaagtcaaagcgtcccctccccatcacaatgataggagacgcagctcacttgatgcctccatttgctggccagggggttaatagcggcttggtagatgctctcatactcagtgacaatttggccgacggtaaattcaattctattgaagaagctgtcaaaaattacgaacagcagatgttcatctacgggaaggaggcacaagaggagtccactcagaatgagatagagatgttcaaaccagatttcaccttccaacaactcttaaacgtctaa

SEQ ID NO: 50 - DNA, 1167 bp, Artificial
  Codon Optimized for Spodoptera Frugiperda
atgacgatgaggatagacacagataagcaaatgaatcttttgagtgataaaaacgtcgcaattatcggtggtggaccagtcggactcacaatggctaagctgctccaacagaacggcatcgacgttagcgtgtatgagcgcgataatgatagggaagcaagaattttcggaggtacgctggacttgcataagggatcaggccaagaggcgatgaaaaaggctggtttgcttcagacttattacgatctggcacttccgatgggcgtaaacatcgcggacaagaagggaaacatactcagcacaaaaaacgtaaagcctgaaaatcgctttgacaatccggagataaatagaaatgacttgcgtgcgatcctgcttaattctctggagaatgatacggttatctgggatcgtaagttggttatgcttgagcctggaaagaagaagtggactctgactttcgaaaacaaaccaagcgagacagcagacctcgtaatcctcgctaatggaggcatgtctaaggtacgcaagtttgtaaccgatactgaggttgaagagaccggcacctttaacatccaagctgacatacaccaacctgaaattaattgccctggattttttcagttgtgcaatggaaatcgtctcatggccagtcatcaaggtaatctgcttttcgccaatcctaacaataacggagcacttcattttggtattagtttcaaaacgccggatgagtggaagaatcaaacgcaagtcgactttcaaaataggaattcagttgtggatttccttctgaaggagttttcagattgggatgaaagatataaggagctcatacatacgacgttgagcttcgtaggccttgctacaaggatttttcctcttgagaaaccgtggaaatccaaacgtccgctgccaatcacaatgataggagacgcagcacatctgatgccgccattcgcaggccaaggagtaaacagcggattggtagacgctttgatcctttccgataatctggccgatggtaagttcaattctatagaagaggcggtgaagaactatgagcaacagatgttcatttatggcaaagaagcacaagaagaatcgacacagaacgaaatcgagatgttcaagcctgacttcaccttccaacaactgttgaacgtataa

SEQ ID NO: 51 - DNA, 1167 bp, Artificial
  Codon Optimized for Strongylocentrotus Purpuratus
atgactatgcgcatcgacacagacaagcagatgaatctactcagcgataaaaacgtggcaataattggtggtggccccgtaggtctgacgatggcaaagctattgcaacaaaacggaatcgatgtgagtgtctatgagagggacaatgacagagaggcacgtatattcggaggtactcttgacctacataagggatcgggccaagaggctatgaaaaaggcaggcctactacaaacgtattacgatcttgccctacctatgggtgttaatatagctgataaaaaaggaaatatcctgagtacaaagaacgtaaagccagagaaccgcttcgataaccccgagataaataggaatgacctgcgagctattcttctcaattcccttgagaacgataccgtgatctgggatcgcaaactggtcatgttggaacccggtaaaaagaagtggactctgacgtttgaaaataaaccctctgaaaccgcagaccttgtgatcctggctaatggtggtatgtccaaagtcagaaagttcgtaactgacacggaggtagaggagaccggcacattcaatatacaggctgacattcaccaaccggagatcaattgcccaggcttctttcagctctgcaatggtaatagactgatggccagtcaccagggcaacttgctctttgcaaaccccaacaataatggtgcccttcattttggcatatcgttcaaaacgccagatgaatggaaaaatcagacgcaagttgattttcagaatcgcaacagtgtggtagacttccttttgaaggaattctcagattgggatgaacgctataaggaactaatacacactacgctttcctttgtcggtctagcaacgagaattttcccgcttgaaaagccttggaaatcaaaaagaccgctaccgataaccatgattggagatgcagctcacctgatgcccccgtttgcaggacaaggcgtgaactctggcctcgtagacgcactcatcctgagcgacaatttggcagacggcaaattcaattcgatagaagaagccgttaaaaactatgaacaacagatgtttatttatggcaaggaggcccaagaagaaagcactcagaacgaaatcgagatgtttaaacctgattttacattccagcaactcctaaatgtgtaa

SEQ ID NO: 52 - DNA, 1167 bp, Artificial
  Codon Optimized for Sus Scrofa
atgacgatgcggattgatacggataaacagatgaaccttctgtctgacaagaacgttgccataatagggggtggtcccgtgggcctgacgatggctaagctgctgcagcagaacgggatagacgtctctgtgtatgagagagataatgatcgggaagcacggattttcggaggaacactcgatttgcataaagggagtgggcaagaagccatgaagaaggctggtctcctccagacctattacgatctggccttgccaatgggtgtcaacatagcagataaaaagggaaatatactttcaaccaaaaatgttaaacccgagaaccggttcgataatccagaaatcaataggaatgaccttcgcgcaatcttgctgaactctctggagaacgatactgtgatctgggacagaaagctggtcatgctcgagcctgggaagaagaaatggacactgacatttgagaataagccaagcgaaacagcagacctcgtgattctggccaacgggggaatgtccaaggttaggaaattcgttaccgacacagaagtcgaagagaccggtaccttcaacattcaagcagatatacatcagcccgaaattaattgtccaggcttctttcaactgtgcaacggcaatcgcttgatggcgtcacaccaggggaacctgctgttcgccaacccaaataacaatggagccctgcatttcggaatctcctttaagaccccggatgagtggaaaaatcaaactcaagtggattttcagaacagaaattccgtggttgacttccttttgaaagagttttccgattgggatgaaagatataaggagctgatccacaccacgctttcattcgtcggtctggcgacacgcatatttccccttgagaaaccgtggaagtcaaagcggccccttccgattacgatgatcggcgatgcagcccatcttatgcctccattcgcgggtcaaggtgtcaattcagggctggttgacgctctgatactctcagataatctggctgatggcaagtttaactccatcgaagaggcggttaagaactatgagcagcaaatgtttatctatgggaaagaggcgcaagaagaaagtacccagaatgagatcgagatgtttaagcctgactttacctttcagcaacttctgaacgtgtaa

SEQ ID NO: 53 - DNA, 1167 bp, Artificial
  Codon Optimized for Tetrahymena Thermophila
atgaccatgagaatcgacactgacaaacaaatgaaccttctcagcgacaaaaatgtagccataatcggtggtggaccagttggacttactatggccaagttacttcagcaaaatggaatagatgtatcagtttatgagagagataatgacagagaagcaagaatattcggaggtactttagaccttcataagggaagcggtcaagaagctatgaaaaaggccggactccttcaaacttattatgacttggccttaccaatgggagtaaacattgccgacaagaagggaaatatactttcaaccaaaaacgtcaaacctgaaaaccgtttcgataatcctgaaatcaaccgtaacgaccttcgtgcaatactcttaaactcactcgaaaacgacactgtcatctgggatcgtaagttggttatgttggagcccggtaagaaaaagtggactctcacatttgagaacaaacccagcgagacagccgaccttgtcatccttgcaaacggaggaatgtctaaggtccgtaaatttgtaaccgatactgaagtcgaagagactggaacctttaatattcaggccgacattcatcaacctgaaatcaattgccctggttttttccagttgtgtaacggaaatagattgatggctagccatcagggaaacctcctttttgcaaaccctaacaacaatggagctttgcactttggtattagttttaaaacacccgacgaatggaagaaccaaacacaggttgacttccaaaatagaaattctgtagttgacttcctcctcaaggagttcagtgattgggatgaaagatacaaagagcttattcacacaactctcagtttcgttggtttggctacaagaatatttcctctcgaaaaaccttggaaaagcaaaagacctctccccattactatgattggtgacgctgcacacctcatgcctcctttcgcaggacagggagttaacagcggacttgttgatgccttgatcctctcagataacttggccgatggaaagttcaattctatagaggaagctgtcaagaactacgagcaacagatgtttatttacggtaaggaggctcaggaagagagtacacagaacgaaatagagatgtttaagccagactttactttccaacagttactcaatgtctaa

SEQ ID NO: 54 - DNA, 1167 bp, Artificial
  Codon Optimized for Thalassiosira Pseudonana
atgactatgcgcattgataccgacaagcagatgaatcttcttagcgataagaacgttgctatcattgggggtggtcctgtcggcctcactatggcgaaactccttcagcagaatggtatcgatgtaagtgtatacgaaagggacaatgaccgagaggccaggattttcggcggtactttggacctccacaagggaagtggccaggaagccatgaagaaggcgggcttgctccagacttattatgacctcgccttgcccatgggcgttaatatcgccgacaaaaaaggaaacatcttgtcgaccaagaatgttaagcccgaaaatagattcgacaatcctgaaattaatcgtaacgacttgcgagcgatcctcctcaatagcttggagaatgatacagttatttgggacaggaaactcgtaatgcttgagccggggaagaagaaatggacccttacctttgaaaataagccgtcagagacggcagacttggtaatcctcgctaatggtggaatgtccaaggtgcgtaaatttgttaccgacacagaggttgaagaaaccggtactttcaacattcaggcggatatccatcaacctgaaattaactgcccagggttttttcagttgtgcaatggcaacagactcatggcatctcaccaaggaaatcttttgttcgcaaatccaaataataacggtgcacttcattttggaattagtttcaaaaccccggacgagtggaagaaccagacacaagtagactttcagaacagaaacagcgttgtagatttcttgcttaaggagttttccgactgggacgaacgctataaagaactcatccacactacacttagctttgtgggccttgcgacccgtatctttcctctcgaaaaaccatggaagagtaagcgcccgctccccatcaccatgattggtgatgcggcgcatcttatgccaccgtttgcaggacagggtgtcaacagcggcttggtagatgccttgatcttgtctgataacttggcggatggcaaattcaatagcatcgaagaggccgtgaaaaactacgaacaacagatgttcatttatggcaaagaggcacaggaagaaagtacccagaacgaaatcgaaatgttcaaacccgacttcacatttcaacagttgctcaacgtctaa

SEQ ID NO: 55 - DNA, 1167 bp, Artificial
  Codon Optimized for Toxoplasma Gondii
atgacgatgcgtattgacacagataaacaaatgaaccttctctccgacaagaacgttgctatcattggaggtggacccgttggactcacaatggcaaaactcctgcagcagaatggcatcgacgtttcggtctatgaacgtgacaacgaccgtgaagctcgcatcttcggtggaaccctggacctccataagggctctggtcaagaagccatgaaaaaagccgggctcctccagacttattatgatctcgcattgcccatgggggttaatattgcggataaaaaggggaacattctctccacgaagaacgttaagccggagaaccgattcgacaaccctgaaattaatcgcaacgatctgcgagctattctccttaacagtcttgagaatgatacggttatctgggatcggaagctcgtgatgctggaacccggtaagaaaaaatggacacttacgttcgaaaacaagccttctgaaaccgccgacctggtgatccttgcaaacggcggcatgtccaaagtccgtaaattcgtgaccgataccgaggtggaggaaactgggacgtttaacatccaggccgatatccatcaaccggagattaattgtccaggatttttccagctctgcaatgggaatcgtctgatggcgagccatcagggaaacctgctctttgctaatcctaacaataacggggcgctccatttcggtatctcttttaaaacaccagacgagtggaagaatcaaactcaagtcgactttcaaaatcgaaactctgtggtcgatttcctccttaaagaattttctgactgggacgagcgctataaagagctcattcatacaacgctgtcgttcgtcgggctggcgacgaggatcttcccccttgagaagccatggaaaagcaagagacccttgccaattacgatgatcggagatgcagcacaccttatgccaccgtttgctggacaaggcgtgaattctgggctggtggacgcccttatcctctcggataatttggcggacggtaagtttaatagtattgaagaggctgttaaaaattatgaacaacaaatgttcatttatgggaaagaagctcaggaggaaagcactcagaatgagattgagatgttcaaacccgatttcaccttccagcaattgttgaacgtctaa

SEQ ID NO: 56 - DNA, 1167 bp, Artificial
  Codon Optimized for Trichoplusia Ni
atgacaatgaggatcgacacggataagcaaatgaacttgctgtccgacaagaatgtagcgatcataggtggcggaccagtgggcctcacaatggccaaactcctgcaacagaatggaattgacgttagtgtttatgaaagggacaatgatagggaagctcgtatttttggcggcacgctcgacctccataaaggctcaggccaggaagccatgaagaaggcaggactccttcaaacgtactacgatcttgccctgccgatgggtgttaacattgcagataagaagggaaacattcttagtacgaagaacgtgaaacccgaaaataggttcgataatccagaaatcaacaggaacgaccttcgtgccattttgttgaattccttggaaaatgacactgtaatatgggatcgtaagttggtaatgttggagccaggcaaaaaaaaatggaccctgacgtttgaaaacaagccgtcagagaccgcggacttggtaatactcgcgaatggaggcatgtctaaggttagaaaatttgtaaccgatacagaggttgaggagactggcacgtttaacatacaagcggacatacaccaaccggagatcaactgcccgggcttttttcagctctgtaacggaaatcgccttatggccagtcatcagggtaatctgctgttcgcaaaccccaacaataatggagcgcttcattttggtatatctttcaaaacgcctgatgaatggaagaatcaaacacaagtggactttcaaaacagaaacagcgtagtggatttcttgctgaaagagttttctgactgggacgagcgttacaaggagctcatccacacaaccttgtccttcgtgggactggctactcgtatcttccctcttgaaaaaccctggaaaagcaagcgcccactccctataacgatgattggagatgctgcccacctgatgccgccatttgcgggacaaggagtaaactctggcctcgtcgatgccttgattctcagcgacaacctggcggatggaaaatttaatagcatcgaggaggcggtcaaaaattatgagcaacagatgtttatctacggtaaggaagcgcaggaagagagtacgcaaaacgagattgaaatgttcaaacctgatttcacctttcaacagctgctcaacgtttaa

SEQ ID NO: 57 - DNA, 1167 bp, Artificial
  Codon Optimized for Triticum Aestivum
atgacgatgcgtattgataccgataagcagatgaatttgctctcagacaaaaacgttgccatcattggaggcggacctgttgggcttactatggctaagctgctccagcaaaacggaatcgatgtttctgtgtacgagagggataacgacagggaagcaagaatctttggagggacgttggacttgcataaagggagcgggcaagaagctatgaaaaaagctggactcctgcagacatattatgatcttgctctgcctatgggagtgaatatcgcagataaaaaaggtaatatcctttctacaaagaatgttaagcccgagaaccgctttgacaatccggaaatcaacagaaacgacctgcgggctatactgctgaactcgcttgaaaatgacaccgttatatgggaccgcaagctggttatgctggaaccggggaagaagaagtggactctcactttcgagaacaagccgtcagagacagcagacctggtgattctcgcaaacggtggaatgtcaaaggtcagaaagttcgtcaccgataccgaagtggaggaaactggcacttttaatatccaggctgacatacaccaaccggaaatcaactgtccaggtttctttcagctgtgtaacggcaataggttgatggcgagccaccagggtaacctgcttttcgctaatcctaataataacggcgctcttcacttcgggatttcctttaaaacccccgatgagtggaagaaccaaactcaggtggattttcagaatcgcaattctgtcgtcgattttcttttgaaggagtttagcgactgggatgagaggtataaagaattgattcataccacgttgtctttcgtggggctcgccacccggatcttccccttggagaagccttggaagtccaaacgccctctccccataacgatgatcggggacgcggcccacctcatgcctcccttcgcagggcagggcgtcaactcaggacttgtcgacgcactgattctttcggacaatcttgctgacggtaagttcaattccatagaggaggcggtgaagaactatgagcagcagatgttcatatacggcaaagaagctcaggaggagagcactcagaacgagatcgagatgttcaaaccggatttcaccttccaacaacttttgaacgtgtaa

SEQ ID NO: 58 - DNA, 1167 bp, Artificial
  Codon Optimized for Trypanosoma Brucei
atgaccatgcgtatcgatacggataaacagatgaaccttctgagcgacaagaatgttgccataataggcgggggtcccgtaggtttgactatggcgaagcttcttcagcaaaacgggattgacgttagcgtttatgaacgggataatgatcgagaagcgcgaatatttggaggtacactggaccttcacaagggctctgggcaagaggccatgaagaaggctggactgttgcagacctactacgatttggctctgcctatgggcgttaatattgctgataagaagggaaacattctcagcaccaaaaatgtcaaacctgaaaaccggtttgataacccagaaattaacaggaatgaccttagggctatcctgctcaactcactcgaaaatgatacagttatctgggaccgcaaactcgtgatgttggagcccggcaagaagaagtggaccctgactttcgaaaataaaccatcagaaactgctgacctggtaattctcgcgaatgggggaatgtcaaaagtacgcaagtttgtaaccgacaccgaggtggaagagactggcacttttaatatacaagcggatatacatcaaccggaaataaattgtccgggattctttcaactctgcaacggtaaccgccttatggcatcgcatcagggaaacctcctgtttgcgaaccctaataataatggagccctccactttggaatatcctttaaaacacctgatgaatggaagaatcagactcaggtcgattttcagaatcggaatagtgtagtagacttccttctgaaagagttttcagactgggatgagcggtataaggaactgatacacacgactttgagttttgtcggtctcgcgacgaggatcttcccactggagaagccttggaagagtaagcgcccgcttccaatcaccatgataggtgacgcagcccaccttatgccccctttcgcgggacaaggggttaattccggtctcgtagatgcccttatactctcagataacctcgctgatggaaaattcaactccatcgaagaagctgtaaagaactatgagcaacagatgttcatctacggcaaagaagcccaggaagagagtacacaaaacgaaattgagatgttcaaacctgactttacctttcagcaactcctcaacgtttaa

SEQ ID NO: 59 - DNA, 1167 bp, Artificial
  Codon Optimized for Trypansoma Cruzi
atgaccatgcgcatagacacagacaaacaaatgaacctgctttctgacaagaacgtggctattattggcggggggcctgtcgggttgaccatggccaagctcctgcagcagaatggaatagatgtgagtgtctatgaacgcgataacgacagggaggcccgtatattcggtgggacactcgacctccacaagggctccggccaggaggccatgaagaaggccggactgcttcagacctattatgaccttgcgttgcccatgggcgtgaacatagcagataaaaaaggcaatatcttgtctactaaaaacgtcaagcccgaaaaccgtttcgataaccctgaaattaatcggaacgacttgcgtgctattctccttaactctttggaaaatgacaccgtcatttgggacaggaagctcgtgatgctcgaaccagggaaaaaaaaatggaccttgacatttgaaaacaaaccctctgaaactgcggaccttgttattctcgccaatgggggaatgtccaaggtgaggaagttcgtcacggataccgaagtcgaagagacgggtactttcaatatccaggcagatatacatcaaccagagataaactgtccgggattctttcaactgtgtaacggaaatcggctcatggcctcgcaccagggcaatcttctgttcgcaaatcccaataacaacggagcactccattttgggatctcatttaagacgccagacgaatggaaaaatcaaacccaagtggactttcaaaaccgcaattcggtggttgattttttgttgaaggagttttcagattgggatgaacgctacaaggagcttattcatacgacgttgtcattcgtggggcttgctacacgcatatttcccctcgagaaaccctggaagtctaagcgaccgctgccgatcacgatgataggcgatgcagcacaccttatgccaccgtttgcaggacagggggttaattctggacttgtcgacgccctcatactgtctgacaacctcgcagacgggaagtttaactccattgaggaggccgttaagaattatgaacaacagatgttcatttacgggaaagaggcacaagaggaatctacccaaaacgaaatcgagatgttcaaaccggacttcacgttccaacagctcctcaatgtctaa

SEQ ID NO: 60 - DNA, 1167 bp, Artificial
  Codon Optimized for Ustilago Maydis
atgaccatgcgcatcgatacggacaaacaaatgaacttgctcagcgataagaatgtcgcaattattggcggcggacctgtcggactgaccatggctaaacttctccaacagaatggtatcgatgtttctgtttacgagcgcgataatgatcgcgaagcgcgaatcttcggaggaacgttggaccttcataaaggctctggtcaggaggcaatgaagaaggcgggattgctccagacatactacgacctcgcattgccaatgggagtcaatattgcagataaaaaaggcaacatcttgtccaccaaaaatgttaagccagagaatcgatttgataatcccgaaatcaaccgtaacgatctccgcgcaatcctcttgaattccttggagaatgatactgtgatctgggaccgtaagcttgtcatgttggaaccgggaaagaagaaatggacattgacttttgaaaacaaaccatccgagacggccgacctggtcatcctcgccaatggtggcatgtcgaaagtgcgtaagtttgtgaccgatacggaggttgaagaaacaggcacttttaatattcaagccgacattcaccagccggaaattaattgtcccggcttttttcagttgtgtaatggaaaccgtcttatggcctctcatcaaggcaatctgctctttgctaatcccaacaataatggcgcgttgcactttggaatttcctttaaaacgcctgatgaatggaagaaccagacgcaagtcgatttccagaaccgtaactccgtggtcgattttctcctgaaagagttcagcgattgggacgagcgatataaagagcttatccatacgacactttcgttcgtcggtcttgcaacccgcattttccccttggagaaaccttggaaatctaaacgcccgctccctatcactatgatcggagacgccgcccacctgatgccaccattcgcaggtcaaggcgttaattcgggcctcgtggacgcgctcatcttgtccgataacttggcggacggtaagttcaactctatcgaggaagctgtcaaaaattacgagcagcaaatgtttatttacggcaaagaggcgcaagaagaatcgacacaaaatgaaatcgagatgttcaagccggatttcactttccaacagcttctgaatgtttaa

SEQ ID NO: 61 - DNA, 1167 bp, Artificial
  Codon Optimized for Xenopus Laevis
atgactatgcgaatagatactgataaacagatgaatctcctctcagacaaaaacgtggccataattggaggaggccctgttggactcaccatggctaaacttctgcaacaaaacgggatagacgtatccgtttacgaacgggataatgaccgcgaagctaggatattcggaggcactttggaccttcacaaagggtccggacaggaggctatgaagaaggctggcctgctccaaacttattatgatttagctcttccaatgggagttaatatcgctgataaaaaaggcaatattttgtctacaaaaaatgtgaagcccgaaaaccggttcgataatcccgaaataaatcgcaacgacttacgtgccattttgctcaacagcttggagaatgacactgttatctgggaccggaagttggttatgttagaacccggaaaaaaaaaatggaccctcacttttgagaacaaaccttctgaaactgcagatctggtaatattggccaatggagggatgtcaaaagtcaggaagttcgttacagacactgaagtagaggaaactggcaccttcaacatccaagctgacatacatcaaccagagataaattgtcccggctttttccagctctgtaacggcaatcggctcatggcatctcatcaaggcaacctgttgtttgcaaaccctaataacaacggggcattgcattttggtatttcctttaaaacacccgatgaatggaaaaatcagacccaggttgacttccaaaacagaaacagtgtggtagattttctgttgaaagagttctccgattgggatgagcgatataaagaactcatccacaccactctttcttttgtcggattagccactcgcatatttcctttggaaaagccctggaagtccaagcgtccattgccaataaccatgatcggtgatgctgctcatttgatgccacccttcgctggacaaggggtaaactcaggacttgtggatgcactcatcctttctgacaatctcgctgacggaaaattcaactcaatcgaagaagccgtgaagaactacgagcagcagatgtttatatatggtaaggaagctcaggaggagagtacacaaaacgaaatagaaatgttcaaaccagattttactttccagcaacttttgaacgtctaa

SEQ ID NO: 62 - DNA, 1167 bp, Artificial
  Codon Optimized for Zea Mays
atgacgatgaggattgacacggacaagcagatgaacttgctgagcgacaaaaatgtcgctatcattggaggaggccccgtgggcttgacgatggcgaaactcctgcaacaaaatgggatagacgtttctgtctatgagagggataatgaccgcgaggcacgcattttcgggggtacattggacctccacaaaggctccgggcaggaagctatgaagaaggcaggtctcctgcagacctattacgatctggcgctgccgatgggggttaacatcgcggataaaaaaggaaatattctctcaaccaaaaatgtgaaaccggagaacagattcgataacccagagattaataggaatgatctgcgggcaattcttctcaactctcttgaaaatgatactgtgatctgggatcggaagctcgtgatgctcgaacccggcaagaaaaaatggacgcttacatttgagaacaagccgagcgaaacagctgacctggtgattctcgcaaacgggggaatgtccaaggttaggaagttcgttacggacacggaagtcgaagagactggaacattcaatattcaggccgatattcatcagcccgagataaactgtccgggcttcttccaattgtgcaatggaaacagacttatggcttcgcatcaaggaaatttgttgtttgctaatcctaataacaatggagcacttcattttggtatatctttcaaaacaccggatgagtggaagaaccaaacgcaagtcgattttcagaaccgcaatagcgtcgtcgacttcctcctcaaggaattttccgattgggatgaacgctacaaagaattgatacatactacgctcagctttgtgggcctggccacgcgcatcttccctcttgagaaaccctggaagtcgaagaggcctcttcctatcacgatgattggtgatgcagcgcacctgatgcctcctttcgcaggacaaggagtcaatagcggtcttgttgatgcactcatactgagcgacaatcttgctgacggaaaatttaattcgatcgaggaagctgtgaaaaattacgagcagcaaatgttcatatatgggaaagaggctcaagaggagagcactcaaaacgagatcgagatgtttaagccggatttcacctttcagcaactgctgaacgtttaa

SEQ ID NO: 63 - protein, 388 aa, Bacteroides fragilis
MTMRIDTDKQMNLLSDKNVAIIGGGPVGLTMAKLLQQNGIDVSVYERDNDREARIFGGTLDLHKGSGQEAMKKAGLLQTYYDLALPMGVNIADKKGNILSTKNVKPENRFDNPEINRNDLRAILLNSLENDTVIWDRKLVMLEPGKKKWTLTFENKPSETADLVILANGGMSKVRKFVTDTEVEETGTFNIQADIHQPEINCPGFFQLCNGNRLMASHQGNLLFANPNNNGALHFGISFKTPDEWKNQTQVDFQNRNSVVDFLLKEFSDWDERYKELIHTTLSFVGLATRIFPLEKPWKSKRPLPITMIGDAAHLMPPFAGQGVNSGLVDALILSDNLADGKFNSIEEAVKNYEQQMFIYGKEAQEESTQNEIEMFKPDFTFQQLLNV

SEQ ID NO: 64 - protein, 388 aa, Artificial
  TETX encoded by codon optimized tetx of SEQ ID NO 3
MTMRIDTDKQMNLLSDKNVAIIGGGPVGLTMAKLLQQNGIDVSVYERDNDREARIFGGTLDLHKGSGQEAMKKAGLLQTYYDLALPMGVNIADKKGNILSTKNVKPENRFDNPEINRNDLRAILLNSLENDTVIWDRKLVMLEPGKKKWTLTFENKPSETADLVILANGGMSKVRKFVTDTEVEETGTFNIQADIHQPEINCPGFFQLCNGNRLMASHQGNLLFANPNNNGALHFGISFKTPDEWKNQTQVDFQNRNSVVDFLLKEFSDWDERYKELIHTTLSFVGLATRIFPLEKPWKSKRPLPITMIGDAAHLMPPFAGQGVNSGLVDALILSDNLADGKFNSIEEAVKNYEQQMFMYGKEAQEESTQNEIEMFKPDFTFQQLLNV

SEQ ID NO: 65 - protein, 359 aa, Bacteroides thetaiotaomicron
MANLLQQNGIDITVYERDENPKARVWGGTLDLHKNSGQEAMKKVGLLQTYYDLALPMGVNFADEKGNIIATRNPTLENKFDNPEINRNALRKMLLGSLKNDTVVWDRKSIGLEQENGKWLLHFENKPTALADFIIVSNGGMSKIRNFVSDNEVEETGTFIIQGDIPEPETNCPEFYKLCNNNRLMTAHQGNLLVANPFNNGMLTYGVIFKKPEEWNNGKGLDFKPTKSVSEFLTNRFSNWSNEYKELIRSTTFFVGLTIKIFPLDKKPWKSNRPLPITLIGDTAHLMPPFAGQGVNIGLMDALILSENLTNGKFGTIQSAIDDYEQRMFVYATEAQADSTKNEIEMRNPSFTFQQLMNV

SEQ ID NO: 66 - protein, 378 aa, Unknown
  Actinoplanes sp. ATCC 31044
MRPRIAVVGAGPGGLSFARVMHHHGHCVTVLERDSGPDARPPGGTLDLHEGMGQVALEKAGLLPEFEKLSRPEGQAMRILAADGTVLRDWRPRPDERANPEIDRGQLRDLLIGPLDVRWGHAVSGVVPGAVHFADGRRESFDLVVGADGAWSRVRPAVSPVTPHYTGVTVVETALDDVDTRHPELAGLIGDGSVGAYGVNRSIVAQRNSGGHVKVSARFRAPLDWHAGLDLTDAAAVRATLLARFDGWAAPVLDLVRRGTAFVHRPIHVLPVGHTWAHGPGVTLLGDAAHLMPPLGAGANLAMLEGAELAEAVAAGPEDLDGVVRTFEERMWARAGMWARITTAGLERLVSADPAEAVAQFDRVNQDSGARTGRCPAA

SEQ ID NO: 67 - protein, 378 aa, Pseudomonas aeruginosa
MTLLKYKKITIIGAGPVGLTMARLLQQNGVDITVYERDKDQDARIFGGTLDLHRDSGQEAMKRAGLLQTYYDLALPMGVNIVDEKGNILTTKNVRPENRFDNPEINRNDLRTILLNSLQNDTVIWDRKLVTLEPDKEKWILTFGDKSSETADLVIIANGGMSKVRKFVTDTEVEETGTFNIQADIHQPEVNCPGFFQLCNGNRLMAAHQGNLLFANPNNNGALHFGISFKTPDEWKSKTQVDFQDRNSVVDFLLKKFSDWDERYKELIRLTSSFVGLATRIFPLDKSWKSKRPLPITMIGDAAHLMPPFAGQGVNSGLMDALILSDNLTNGKFNSIEEAIENYEQQMFAYGREAQTESIINETEMFSLDFSFQKLMNL

SEQ ID NO: 68 - protein, 380 aa, Bacillus amyloliquefaciens
MNSQEKRIAIIGAGPGGLTLARILQQGGLAPVIYEQETSPAERQQGGTLDLDEQTGQKALQAAGLLGSFRSICRYEGQALKITDKKGTVFAETEPEKLTDHGRPEIDRTELRRLLLQSLKTDTIKWGHKLSHAVPLEKGGHKLEFENGHTDVFDLIIAADGAFSRVRPLLSDAPVEYSGISMIELHIMNAAADFPDLAGFHGTGSMYALDDRKAIMAQLNGDGTVRVYLCFAAGRYWIDENDIDYDQPEEAKQKLLELFEDWSDDLKHYIQYAGETILPRRLYSLPVQHKWEHKQGVTLIGDAAHLMTPFAGAGANLTMLDAAELGLSILHNADTDKAVKQYEEKMFAYAEETAAETGSHMKTFFSESAAQKIGAMMNAF

SEQ ID NO: 69 - protein, 381 aa, Corallococcus coralloides
MHIAKTVGIIGGGPGGLTLARILETRGIAATVFELDEHPFARPQGGSLDLHGDSGLRALREAGLEAGFKAVARYDDQGDAIYDAQGTLHFQHNEASDGDRPEIDRTQLRSLLLDSLPAEWLRWGSKVSAVESLSDGRYRVMGPAGTLGEFDLVVGADGAWSRIRPLVSSATPAFTGVLFIELQIDDVDTRHPEVAKLLPRGKISVVGNNQGLIAQRSSHGHVRAYFMFRVSEAQLQQGLVDTSSPTRAREQLKALLPGWAPSLLTFIDACNDSIVARPIVALPVGHRWMHRPGVTLLGDAAHVMPPFSGEGVNMAMLDGLELGLALAADADWSRAVKGYEEAMFERAASAAAGAMQGLDFVSEHALEHVLEHFRELQQAGA

SEQ ID NO: 70 - protein, 383 aa, Unknown
  Actinoplanes sp. ATCC 31044
MTTPTISIVGAGLGGLTLARVLHVHGIASTIYELDASPTARTQGGMLDIHDYNGQPALHEAGLFEQFRGIIHVGGEATRVLDSRNVVHLDEPDDGTGGRPEVERGQLRDILLDSLPAGTIRWGMKVTGVRTLDGGRHELTFADGTTVVTDVLVGADGAWSRIRPLVSTATPSYAGVSFVELDLFDADERHPGPAEVVGSGGLMALGPGKGILAHREPDDSLHVYVAVRTSPDWLPGIDFSDTATAKAAVIEQFADWAPELQAVIAEADSPLTPRMVNMLPIEHRWDRVAGVTLLGDAAHLMSPFAGEGANLAMFDGAELGKAIAAHPGDIEAALTSYEKGLFPRSAEAAAEANRNLEICFADDAPQSLLKLFAGYAAAAESTH

SEQ ID NO: 71 - protein, 385 aa, Flavobacterium psychrophilum
MKNNLAENKKIAIIGGGPVGLTTAILLQQKGVNVKVYERDLNAQTRISGGTLDIHNDTGQLAFKKAGLLELFFKNARPTGERAVDIQANIVEEVMPTEENKLERPEIDRNDMRRILLESLNENTVEWDSQLINLEKKENQFHLQFKNGKIEIADVVIIENGGQSNARKYVTDLTPKYTGTFVLQGEVLNPEIFCPNYKNLCKEENTMTISDRKMLFCQVKAKGALNYYLSFKADEDWAVKANIDLNNKESIVAFMNEKCANWHPTFKELFAATDNFTSLAMRMLNVENGWKSKETNITLVGDAAHLMPPFAGVGVNVGLLDALNLAINLTEGDFSNIDAAIKDYEQKMFVYAAEAQDGTSQAEEGIHSDISFEELMKQREEGHRK

SEQ ID NO: 72 - protein, 386 aa, Unknown
  uncultured bacterium HH1107
MRIDTDKQMNLLSDKNVAIIGGGPVGLTMAKLLQQNGIDVSVYERDNDREARIFGGTLDLHKGSGQEAMKKAGLLQTYYDLALPMGVNIADEKGNILSTKNVKPENRFDNPEINRNDLRAILLNSLENDTVIWDRKLVMLEPGKKKWTLTFENKPSETADLVILANGGMSKIRSFVTDTQVEETGTFNIQADILQPEINCPGFFQLCNGNRLMAGHQGILLFANPNNNGALYLGISFKTPDEWKNKIPLDFQDRNSVADFLLKRFSKWSEVYKQLIRSVSTFQCLPTRKFPLNNDWKSNRPLPITMIGDAAHLMSPFAGQGVNTGLLDALILSENLTNGEFTSIENAIENYEQQMFVYAKDTQDESTENETEMFSPNFSFQKLLNL

SEQ ID NO: 73 - protein, 388 aa, Bacteroides fragilis; identical to SEQ ID NO: 64

SEQ ID NO: 74 - protein, 388 aa, Bacteroides thetaiotaomicron
MTMRIDTDKQMNLLSDKNVAIIGGGPVGLTMAKLLQQNGIDVSVYERDNDREARIFGGTLDLHKGSGQEAMKKAGLLQTYYDLALPMGVNIADEKGNILSTKNVKPENRFDNPEINRNDLRAILLNSLENDTVIWDRKLVMLEPGKKKWTLTFENKPSETADLVILANGGMSKVRKFVTDTEVEETGTFNIQADIHQPEINCPGFFQLCNGNRLMASHQGNLLFANPNNNGALHFGISFKTPDEWKNQTQVDFQNRNSVVDFLLKEFSDWDERYKELIHTTLSFVGLATRIFPLEKPWKSKRPLPITMIGDAAHLMPPFAGQGVNSGLVDALILSDNLADGKFNSIEEAVKNYEQQMFIYGKEAQEESTQNEIEMFKPDFTFQQLLNV

SEQ ID NO: 75 - protein, 388 aa, Riemerella anatipestifer
MTMRINTDKQMNLLSDKNVAIIGGGPVGLTMAKLLQQNGIDVSVYERDNDREARIFGGTLDLHKGSGQEAMKKAGLLQTYYDLALPMGVNIADEKGNILSTKNVKPENRFDNPEINRNDLRAILLNSLENDTVIWDRKLVMLEPGKKKWTLTFENKPSETADLVILANGGMSKVRKFVTDTEVEETGTFNIQADIHQPEINCPGFFQLCNGNRLMASHQGNLLFANPNNNGALHFGISFKTPDEWKNQTQVDFQNRNSVVDFLLKKFSDWDERYKELIHATLSFVGLATRIFPLEKPWKSKRPLPITMIGDAAHLMPPFAGQGVNSGLVDALILSDNLADGKFNSIEEAVKNYEQQMFIYGKEAQEESTQNEIEMFKPDFTFQQLLNV

SEQ ID NO: 76 - protein, 388 aa, Riemerella anatipestifer
MTMRIDTDKQMNLLSDKNVAIIGGGPVGLTMAKLLQQNGIDVSVYERDNDREARIFGGTLDLHKGSGQEAMKKAGLLQTYYDLALPMGVNIADEKGNILSTKNVKPENRFDNPEINRNDLRAILLNSLENDTVIWDRKLVMLEPGKKKWTLTFENKPSETADLVIIANGGMSKVRKFVTDTEVEETGTFNIQADIHQPEVNCPGFFQLCNGNRLMAAHQGNLLFANPNNNGALHFGISFKTPDEWKSKTRVDFQDRNSVVDFLLKKFSDWDERYKELIRLTSSFVGLATRIFPLDKSWKSKRPLPITMIGDAAHLMPPFAGQGVNSGLMDALILSDNLTNGKFNSIEEAIENYEQQMFIYGKEAQEESTQNEIEMFKPDFTFQQLLNV

SEQ ID NO: 77 - DNA, 316 bp, Chlamydomonas reinhardtii
agctctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaac

SEQ ID NO: 78 - DNA, 230 bp, Chlamydomonas reinhardtii
gggacctgatggtgttggtggctgggtagggttgcgtcgcgtgggtgacagcacagtgtggacgttgggatccggcaagactggccccgcttggcaacgcaacagtgagcccctccctagtgtgtttggggatgtgactatgtattcgtgtgttggccaacgggtcaacccgaacagattgatacccgccttggcatttcctgtcagaatgtaacgtcagttgatggtac

SEQ ID NO: 79 - DNA, 653 bp, Chlamydomonas reinhardtii
agctcgctgaggcttgacatgattggtgcgtatgtttgtatgaagctacaggactgatttggcgggctatgagggcgggggaagctctggaagggccgcgatggggcgcgcggcgtccagaaggcgccatacggcccgctggcggcacccatccggtataaaagcccgcgaccccgaacggtgacctccactttcagcgacaaacgagcacttatacatacgcgactattctgccgctatacataaccactcagctagcttaagatcccatcaagcttgcatgccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaaatggccaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcagg

SEQ ID NO: 80 - DNA, 380 bp, Chlamydomonas reinhardtii
agcttgcatgccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaaatggccaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcagg

SEQ ID NO: 81 - DNA, 30 bp, Artificial
  Primer sequence of tetXhF
tctcgagatgaccatgcgcatcgacaccga

SEQ ID NO: 82 - DNA, 31 bp, Artificial
  Primer sequence of tetBaR
tggatcctcacacgttcagcagcagctgctg

SEQ ID NO: 83 - DNA, 19 bp, Artificial
  Primer sequence of aph8F
cgtgcactgcggggtcggt

SEQ ID NO: 84 - DNA, 18 bp, Artificial
  Primer sequence of aph8R
ccgccccatcccacccgc

SEQ ID NO: 85 - DNA, 16 bp, Artificial
  Primer sequence of ble1F
ccgggtcgcgcagggc

SEQ ID NO: 86 - DNA, 18 bp, Artificial
  Primer sequence of ble1R
gcgccgttccggtgctca

SEQ ID NO: 87 - DNA, 234 bp, Chlamydomonas reinhardtii
agcttgcatgccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaaatggccag

SEQ ID NO: 88 - DNA, 622 bp, Artificial Sequence
  Derived from Pichia pastoris Nucleotide sequence of gap promoter
gatctactagtgagctctacgtgcccgggtttttgtagaaatgtcttggtgtcctcgaccaatcaggtagccatccctgaaatacctggctccgtggcaacaccgaacgacctgctggcaacgttaaattctccggggtaaaacttaaatgtggagtaatagaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaangtcccggccgtcgctggcaataactgcgggcggacgcatgtcttgagattattggaaaccaccagaatcgaatataaaaggcgaacacctttcccaattttggtttctcctgacccaaagactttaaatttaatttatttgtccctatttcaatcaattgaacaactattcatcattattagcttactttcataattgcgactggttccaattgacaagcttttgattttaacgacttttaacgacaacttgagaagatcaaaaaacaactaattattcgaaacg

SEQ ID NO: 89 - DNA, 818 bp, Cricetulus griseus
ggcccgggccgtacgaatttatgttacttggcagaggccgcatggaaagtccctggacgtgggacatctgattaatacgtgaggaggtcagccatgttctttttggcaaaggactacggtcattggacgtttgattggcatgggatagggtcagccagagttaacagtgttcttttggcaaagggatacgtggaaagtcccgggccatttacagtaaactgatacggggacaaagcacagccatatttagtcatgtattgcttggcagagggtctatggaaagtccctggacgtgggacgtctgattaatatgaaagaaggtcagccagaggtagctgtgtcctttttggcaaagggatacggttatgggacgtttgattggactgggatagggtcagccagagttaacagtgttcttttggcaaaggaaacgtggaaagtcccgggccatttacagtaaactgatactgggacaaagtacacccatatttagtcatgttctttttggcaaagagcatctggaaagtcccgggcagcattatagtcacttggcagagggaaagggtcactcagagttaagtacatctttccagggccaatattccagtaaattacacttagttttatgcaaatcagccacaaaggggattttcccggtcaattatgactttttccttagtcatgcggtatccaattactgccaaattggcagtacatactaggtgattcactgacatttggccgtcctctggaaagtccctggaaaccgctcaagtactgtatcatggtgactttgcatttttggagagcacgcccca

(19) United States
(12) Patent Application Publication
(10) Pub. No.: US 2017/0015979 A1
(43) Pub. Date: Jan. 19, 2017

(54) TETRACYCLINE RESISTANT EUKARYOTIC CELLS EXPRESSING AN NADP-REQUIRING OXIDOREDUCTASE

(71) Applicant: Instituto Tecnológico y de Estudios Superiores de Monterrey, Nuevo León (MX)

(72) Inventors: Guy A. Cardineau, Nuevo León (MX); Sergio Ángel García Echaurri, Nuevo León (MX)

(21) Appl. No.: 15/099,227

(22) Filed: Apr. 14, 2016

Related U.S. Application Data
(60) Provisional application No. 62/147,089, filed on Apr. 14, 2015.

(51) Int. Cl. C12N 9/02; C12P 21/00; C12N 15/85; C12N 15/82; C12N 15/81

(52) U.S. Cl. CPC C12N 9/0073; C12Y 114/13008; C12N 15/8271; C12N 15/81; C12N 15/85; C12P 21/00

(57) ABSTRACT

TETX, a tetracycline degrading enzyme, is provided as a selection marker for eukaryotic cells. Polynucleotide molecules containing tetx and DNA constructs comprising a tetx under the control of a eukaryotic promoter are provided. Additionally, DNA constructs that contain a gene of interest and a tetx, each under the control of an appropriate promoter are provided. Further, methods of expressing tetx in a eukaryotic cell are provided, the method comprising introducing a vector comprising tetx into eukaryotic cells, culturing the eukaryotic cells in the presence of tetracycline, and identifying and isolating the cell that expresses tetx. Furthermore, a method is provided for expressing a gene of interest encoding a protein of interest in a eukaryotic cell by culturing a cell containing a vector comprising a gene of interest and a tetx, each under the control of an appropriate promoter.

### FIELD OF THE INVENTION

A genetic transformation method for the selection of eukaryotic cells transformed with a nucleotide encoding a tetracycline degrading enzyme, for example, NADP-requiring oxidoreductase, (TETX) is provided. The method comprises introducing a tetx that encodes TETX in a eukaryotic host cell. TETX degrades a broad spectrum of tetracycline antibiotics. Accordingly, a eukaryotic cell expressing TETX and that is resistant to tetracycline is also provided. Vectors containing tetx and codon optimized tetx for TETX expression in a eukaryotic cell, particularly, in an algal cell, are also provided.

### BACKGROUND

The genes frequently used in transformation of eukaryotes are neomycin phosphotransferase, which confers resistance to the aminoglycosides kanamycin and G418; histidinol dehydrogenase, which confers resistance to L-histidinol in a histidine lacking medium; hygromycin phosphotransferase, which confers resistance to hygromycin B; and sh ble gene from Streptoalloteichus hindustanus, which confers resistance to bleomycin-phleomycin antibiotics. These antibiotics are expensive to use in large quantities and the presence of these antibiotics is required to keep the resistant cell lines under constant selection to induce the propagation of the gene conferring antibiotic resistance.

### BRIEF SUMMARY OF THE INVENTION

A tetracycline degrading enzyme, namely, TETX, encoded by tetx and which confers resistance to a tetracycline, is provided as a selection marker for eukaryotic cells. In one embodiment, tetx is identified by Genbank ID: JQ990987 and has the nucleotide sequence of SEQ ID NO: 1 and the amino acid sequence of SEQ ID NO: 63. Codon optimized tetx having the sequence of any one of SEQ ID NOs: 2-62 are also provided.

Thus, various embodiments provide polynucleotide molecules containing tetx having the sequence of any one of SEQ ID NOs: 1-62 or a homolog thereof. A homolog of tetx has at least 70% sequence identity to the sequence set forth in any one of SEQ ID NOs: 1-62 or is a fragment thereof that encodes TETX that degrades a tetracycline.

Other aspects of the invention provide DNA constructs (also referred to as nucleotide constructs) comprising a tetx under the control of a promoter, wherein said promoter drives the transcription of the tetx in a eukaryotic cell. In addition to the tetx under the control of a promoter, the DNA constructs can also contain a gene of interest encoding a protein of interest. The gene of interest can be under the control of an appropriate promoter. DNA Vectors, for example, eukaryotic expression vectors, containing the DNA constructs comprising tetx are also provided.

Methods of expressing tetx in a eukaryotic cell are provided, the method comprising introducing a DNA construct disclosed herein into eukaryotic cells, culturing the eukaryotic cells in the presence of tetracycline, and identifying and isolating the cell that expresses tetx. Accordingly, certain embodiments provide a eukaryotic cell containing the tetx or DNA constructs containing tetx.

Further, a method is provided for expressing a gene of interest encoding a protein of interest, the method comprising the steps of:

a) obtaining a eukaryotic cell having a DNA construct comprising:
i) tetx under the control of a first promoter, wherein the first promoter drives the transcription of tetx, and
ii) the gene of interest encoding the protein of interest, optionally, under the control of a second promoter;

b) culturing the eukaryotic cell under appropriate conditions that cause the expression of the gene of interest.

### BRIEF DESCRIPTION OF THE DRAWINGS

The patent or application file contains at least one drawing executed in color. Copies of this patent or patent application publication, with color drawing(s), will be provided by the Office upon request and payment of the necessary fee.

FIG. 1. Schematic representation of the genetic elements of certain vectors disclosed herein. Three components of the tetx are shown: the promoter, coding region and terminator elements. (I) A promoter or DNA sequence that drives the production of mRNA or acts as an internal ribosomal entry site. (II) The coding sequence of the tetracycline degrading enzyme. (III) A terminator or any DNA sequence that facilitates the end of transcription.

FIG. 2. Example of a promoter, tetracycline degrading enzyme coding sequence and terminator used for the nuclear transformation Chlamydomonas reinhardtii. The BtetX sequence includes the beta 2 tubulin promoter (SEQ ID NO: 77), tetx Open Reading Frame (ORF) (SEQ ID NO: 2) and cop1 3′UTR (SEQ ID NO: 78). In red are primer binding sites for tetXhF and tetBaR.

FIG. 3. Photograph of a 1% TAE-agarose gel showing the products of a colony PCR of tetracycline resistant C. reinhardtii colonies transformed with AtetX or BtetX.

FIG. 4. Tetracycline resistance phenotype. BtetX (5 strains) or AtetX (1 strain) positive transformed C. reinhardtii, and CC-849 as a negative control, were grown in TAP media with (+C) and without antibiotic selection for 26 cell divisions. A 10 μL droplet containing 10⁴ cells of each strain was plated on TAP plates supplemented with either 15, 25, 50 or 100 μg/mL of tetracycline and incubated with a light intensity of 25 μmoles/ms. The tetracycline resistance phenotype is present in all transformed strains. There appears to be a selection for more resistant cells at higher tetracycline concentrations that grow as patches or along the periphery of the absorbed droplet. Negative controls did not grow at any assayed tetracycline concentration.

FIG. 5. Tetracycline killing curve for N. benthamiana leaf explants. The graph shows percentage of explants necrosis and shoots induction in response to different tetracycline concentrations in time.

FIG. 6. Doxycycline killing curve for N. benthamiana leaf explants. The graph shows percentage of explants necrosis and shoots induction in response to different doxycycline concentrations.

### BRIEF DESCRIPTION OF THE SEQUENCES

SEQ ID NO: 1: Nucleotide sequence of coding region of wild-type tetx from Enterobacteriaceae bacterium SL1.

SEQ ID NO: 2: Nucleotide sequence of codon optimized tetx for the expression in C. reinhardtii cytoplasm.

SEQ ID NOs: 3-62: Sequences of codon optimized tetx for the expression in various eukaryotic cells as indicated in Table 1.

SEQ ID NO: 63: Amino acid sequence of wild-type TETX from Enterobacteriaceae bacterium SL1.

SEQ ID NO: 64: Amino acid sequence of TETX encoded by the codon optimized tetx of SEQ ID NO: 3.

SEQ ID NOs: 65-76: TETX sequences associated with Uniprot entry numbers Q93L50, G8SBP9, Q7X2A0, E1UR95, H8MZP2, R4LB07, A6GZT8, B5TTM2, Q01911, Q93L51, E5D2K6, A0A0H4JF53, respectively.

SEQ ID NO: 77: Nucleotide sequence of beta 2 tubulin promoter.

SEQ ID NO: 78: Nucleotide sequence of cop1 3′ untranslated region.

SEQ ID NO: 79: Nucleotide sequence of hsp70a:rbcsc2 promoter.

SEQ ID NO: 80: Nucleotide sequence of rbcs2 intron.

SEQ ID NO: 81: Primer sequence of tetXhF.

SEQ ID NO: 82: Primer sequence of tetBaR.

SEQ ID NO: 83: Primer sequence of aph8F.

SEQ ID NO: 84: Primer sequence of aph8R.

SEQ ID NO: 85: Primer sequence of ble1F.

SEQ ID NO: 86: Primer sequence of ble1R.

SEQ ID NO: 87: Nucleotide sequence of rbcs2 promoter.

SEQ ID NO: 88: Nucleotide sequence of yeast gap promoter.

SEQ ID NO: 89: Nucleotide sequence of Cricetulus griseus chpv2 promoter.

### DETAILED DESCRIPTION

As used herein, the singular forms “a”, “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof) “comprising,” “comprises,” “comprise,” “consisting essentially of,” “consists essentially of,” “consisting,” and “consists” can be used interchangeably.

The term “about” or “approximately” means within an acceptable error range for the particular value as determined by one of ordinary skill in the art, which will depend in part on how the value is measured or determined, i.e., the limitations of the measurement system. In the context of a quantitative aspect where the term “about” or “approximately” is used, the relevant aspect can be varied by ±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc.

As used herein, the name of a gene is written in lower case and italicized font and the word “gene” may not be spelled out; whereas, the name of a protein is written in capital letters and regular font. For example, the term “tetx” (lower case and italicized) indicates “tetx gene,” and the term “TETX” indicates TETX protein.

The term “promoter” refers to a regulatory region of DNA usually comprising a “TATA box” capable of directing RNA polymerase II to initiate RNA synthesis at the appropriate transcription initiation site for a particular coding sequence. A promoter can additionally comprise other recognition sequences generally positioned around the TATA box, referred to as promoter elements, which influence the transcription initiation rate. Certain promoter elements may also be present downstream of the transcription start site. Promoter elements that enable transcription can be identified, isolated, and used in the embodiments described herein.

An inducible promoter is a promoter capable of directly or indirectly activating transcription of one or more DNA sequences or genes in response to an inducer. The inducer can either be a chemical agent, such as a metabolite, growth regulator, herbicide, or a phenolic compound, or a physiological stress imposed on a cell, such as cold, heat, nutrient starvation, etc. For a well-controlled expression of a target gene, it is highly desirable to express the gene using tightly regulated stress or chemically-inducible promoters. A gene under control of a tightly regulated inducible promoter is not transcribed or has very low transcription in the absence of an inducer whereas the gene is transcribed at a high level only when the inducer is present.

For the purpose of this invention, the term “when the inducer is present” indicates that the inducer is present in the cell at sufficient concentration or the inducer condition is experienced by the cell to induce the expression of the gene under the control of the inducible promoter.

A promoter when assembled within a DNA construct such that the promoter is operably linked to a gene enables transcription of the gene in a cell containing the DNA construct. The term “operably linked” is intended to mean that the transcription of the gene is under the influence of the promoter. “Operably linked” is also means that the joining of two nucleotide sequences is such that the coding sequence of each DNA fragment remains in the proper reading frame.

An expression vector indicates that the vector contains appropriate promoter elements and coding regions of a gene to express a protein in a host. A host can be a prokaryote or a eukaryote. A prokaryotic expression vector induces the expression of the gene contained therein in a prokaryotic cell; whereas, a eukaryotic expression vector induces the expression of the gene contained therein in a eukaryotic cell. Certain vectors can be used in both prokaryotic and eukaryotic cells; whereas, certain other vectors can be specific for a prokaryotic or a eukaryotic cell.

The term “heterologous promoter” indicates a promoter sequence that is not naturally present in a host cell operably linked to a gene. While this promoter sequence is heterologous to the gene, it can be homologous, or native; or heterologous, or foreign, to the host cell.

As used herein, the term “transformed cell” or “genetically engineered cell” refers to a cell that comprises within its genetic material tetx which is not naturally present in the cell. The tetx can be stably incorporated within the chromosome of a cell such that the gene is passed on to successive generations via the cell's chromosomes. The tetx can also remain as an extra-chromosomal genetic material, such as a plasmid, and passed on to successive generations as extra-chromosomal genetic material. Multiple copies of tetx can be present in a cell where the cell also naturally contains one copy of tetx.

As used herein, the term “eukaryotic cell” includes a cell which contains a nucleus and may contain other cellular organelles. Non-limiting examples of eukaryotic cells include the cells from animals (such as mammalian and insert cells), plants, fungi (such as filamentous and non-filamentous fungi), protozoa, and algae. In certain embodiment, the eukaryotic cell is an algal cell, for example, C. reinhardtii or Synechococcus elongates. In further embodiments, the eukaryotic cell is a plant cell, such as but not limited to, for example, a plant cell from soybean, corn, tomato, cotton, canola, tobacco, rice, peppers, sugar beets, alfalfa, and potatoes. Additional examples of appropriate eukaryotic cells in which the methods described herein can be practiced are known to a person of ordinary skill in the art and such embodiments are within the purview of the invention.

The term “tetracycline” refers to an antibiotic from the tetracycline family of antibiotics. The antibiotics from the tetracycline family are broad-spectrum antibiotics, exhibiting inhibitory activity against a wide range of gram-positive and gram-negative bacteria, atypical organisms such as chlamydiae, mycoplasmas, rickettsiae, and protozoan parasites. The antibiotics belonging to the tetracycline family contain a linear fused tetracyclic nucleus to which a variety of functional groups are attached. The simplest tetracycline to display detectable antibacterial activity is 6-deoxy-6-demethyltetracycline. Non-limiting examples of tetracyclines include Tetracycline, Chlortetracycline, Oxytetracycline, Demeclocycline, Lymecycline, Meclocycline, Methacycline, Minocycline, Rolitetracycline, doxycycline, and Tigecycline. Additional examples of antibiotics from the tetracycline family are known to a person of ordinary skill in the art and such embodiments are within the purview of the invention.

A “nucleotide” as used herein refers to a single nucleotide, for example, a single ribonucleotide or deoxyribonucleotide, for example, adenine, thymine, cytosine, guanine, and uracil; or a single base-paired nucleotide, for example, a based-paired ribonucleotide or a based-paired deoxyribonucleotide, for example, a base-paired adenine:thymine, cytosine:guanine or adenine:uracil.

A “polynucleotide molecule” as used herein refers to a polynucleotide having a specific function, for example, coding region of a protein or a promoter which drives the expression of an operably linked gene. A DNA construct as used herein refers to a polynucleotide having two or more polynucleotide molecules, for example, a promoter operably linked to a gene. A vector as used herein refers to a polynucleotide containing one or more DNA constructs and/or polynucleotide molecules further comprising an appropriate origin of replication, sites of recombinant integration into a host cell chromosome, etc. Various vectors that can be used in the claimed invention are known to a person of ordinary skill in the art and such embodiments are within the purview of the invention.

TETX is a soluble protein and degrades at least the following compounds: chlortetracycline, demeclocycline, doxycycline, minocycline, oxytetracycline, tetracycline, doxycycline, and tigecycline. A eukaryotic cell harboring the tetx exhibits resistance to a variety of tetracyclines.

An embodiment of the invention provides a tetx that encodes a TETX, wherein the TETX degrades a tetracycline. The term “tetx” refers to a nucleotide having the sequence of any one of SEQ ID NOs: 1-62 or a homolog thereof or a fragment thereof that encodes for a TETX that degrades a tetracycline.

In one embodiment, the tetx has the sequence of any one of SEQ ID NOs: 1-62. SEQ ID NO: 1 provides the sequence of tetx from Enterobacteriaceae bacterium SL1; whereas, SEQ ID NO: 2 provides a tetx that is codon optimized for the expression of TETX in C. reinhardtii cytoplasm. SEQ ID NOs: 3-62 provide a number of tetx genes, each of which is codon optimized for the expression of TETX in cells from various organisms as indicated in Table 1 below. A homolog or a fragment of tetx of any one of SEQ ID NOs: 1-62 are also provided.

TABLE 1
SEQ ID NOs of codon optimized versions
of tetx in various eukaryotic cells

| Organism | SEQ ID NO: |
|---|---|
| Drosophila melanogaster | 3 |
| Homo Sapiens (human) | 4 |
| Mus Musculus (mouse) | 5 |
| Pichia Pastoris | 6 |
| Saccharomyces cerevisiae | 7 |
| Arabidopsis thaliana | 8 |
| Aspergillus niger | 9 |
| Bombyx mori (silkworm) | 10 |
| Bos Taurus (bovine) | 11 |
| Danio rerio (zebrafish) | 12 |
| Brassica napus (rape) | 13 |
| Caenorhabditis elegans (nematoad) | 14 |
| Candida albicans | 15 |
| Canis familiaris (dog) | 16 |
| Chlamydomonas reinhardtii | 17 |
| Cricetulus griseus (hamster) | 18 |
| Cyanophora paradoxa | 19 |
| (Glaucophyte Algae) | |
| Dictostelium discoideum | 20 |
| Emericella nidulans | 21 |
| Gallus gallus (chicken) | 22 |
| Glycine max (soybean) | 23 |
| Hordeum vulgare subsp vulgare (Barley) | 24 |
| Kluyveromyces lactis | 25 |
| Leishmania donovani | 26 |
| Macaca fascicularis (macaque) | 27 |
| Manduca sexta (tobacco hornworm) | 28 |
| Medicago sativa (Alfalfa) | 29 |
| Neurospora crassa | 30 |
| Nicotiana benthamiana | 31 |
| (relative of tobacco) | |
| Nicotiana tabacum (tobacco) | 32 |
| Oncorhynchus mykiss | 33 |
| (Rainbow trout) | |
| Oryctolagus cuniculus (rabbit) | 34 |
| Oryza saliva (rice) | 35 |
| Ovis aries (sheep) | 36 |
| Petunia × hybrida | 37 |
| Phaseolus lunatus (lima bean) | 38 |
| Pisum sativum (pea) | 39 |
| Plasmodium falciparum 3D7 | 40 |
| Rattus norvegicus (rat) | 41 |
| Salmo salar (Atlantic salmon) | 42 |
| Schistosoma mansoni | 43 |
| Schizosaccharomyces pombe | 44 |
| Schmidtea mediterranea | 45 |
| Solanum lycopersicum (tomato) | 46 |
| Solanum tuberosum (potato) | 47 |
| Sorghum bicolor | 48 |
| Spinacia oleracea (spinach) | 49 |
| Spodoptera frugiperda | 50 |
| Strongylocentrotus purpuratus (sea urchin) | 51 |
| Sus scrofa (Pig) | 52 |
| Tetrahymena thermophila | 53 |
| Thalassiosira pseudonana | 54 |
| Toxoplasma Gondii | 55 |
| Trichoplusia ni | 56 |
| Triticum aestivum (wheat) | 57 |
| Trypanosoma brucei | 58 |
| Trypanosoma cruzi | 59 |
| Ustilago maydis | 60 |
| Xenopus laevis | 61 |
| Zea mays | 62 |

Certain codon optimized tetx genes can be used for the expression of TETX in other organisms, for example, based on the prediction of suitable hosts for tetx according to Codon Adaptation Index shown in Table 2 below.

TABLE 2
Adaptability of codon optimized tetx in various organisms.

| | Gene | | |
|---|---|---|---|
| | tetx | tetx | tetx |
| Species | Cr | Cg | Yeast |
| Aspergillus niger | 0.96 | 0.86 | 0.76 |
| Arabidopsis thaliana | 0.60 | 0.71 | 0.94 |
| Bombyx mori | 0.91 | 0.89 | 0.94 |
| Cricetulus griseus | 0.88 | 0.90 | 0.68 |
| Drosophila | 0.95 | 0.77 | 0.56 |
| melanogaster | | | |
| Escherichia coli | 0.76 | 0.73 | 0.63 |
| Homo sapiens | 0.90 | 0.88 | 0.65 |
| Nicotiana tabacco | 0.54 | 0.69 | 0.91 |
| Pichia pastoris | 0.55 | 0.68 | 0.94 |
| Saccharomyces | 0.49 | 0.61 | 0.91 |
| cerevisiae | | | |
| Spodoptera frugiperda | 0.78 | 0.73 | 0.80 |
| Zea mays | 0.98 | 0.77 | 0.57 |

Gene variants: Cr—Chlamydomonas reinhardtii,
Cg—Cricetulus griseus,
-Yeast

A homolog of the tetx can be designed based on sequence analysis of any one of SEQ ID NOs: 1-62 and assessing the activity of the protein encoded by the homolog in degrading one or more tetracyclines. In certain embodiments, a homolog shares about 70%-100%, about 75-95%, about 80%-90%, about 85%, or about 95% sequence identity with any one of SEQ ID NOs: 1-62.

A homolog of the tetx encodes a homolog of TETX that exhibits tetracycline degrading activity. A homolog of TETX shares about 70%-100%, about 75-95%, about 80%-90%, about 85%, or about 95% sequence identity with any one of the sequences of SEQ ID NOs: 63-76. Homologs of tetx also include synthetically derived polynucleotide sequences, for example, polynucleotides generated by using site-directed mutagenesis.

A fragment of the tetx can be prepared based on the sequence of any one of SEQ ID NOs: 1-62, and assessing the activity of the TETX encoded by a fragment in degrading one or more tetracyclines. In one embodiment, the fragment of tetx has about 3-60, about 3, about 15, about 30, about 45, or about 60 nucleotides truncation at one or both of the 5′ and 3′ end of any one of SEQ ID NOs: 1-62. Accordingly, in one embodiment, a fragment of tetx encodes a TETX that has about 1-20, about 1, about 5, about 10, about 15, or about 20 amino acids fewer at one or both of the N or C termini of SEQ ID NO: 63 or 64.

In one embodiment, the tetx has the sequence of any one of SEQ ID NOs: 2-62 or is a homolog of any one of SEQ ID NOs: 2-62, and wherein the tetx has a sequence that is different from a naturally occurring tetx. A eukaryotic cell is also provided that comprises the tetx that has the sequence of any one of SEQ ID NOs: 2-62 or a homolog of any one of SEQ ID NOs: 2-62, and wherein the tetx has a sequence that is different from a naturally occurring tetx.

Naturally occurring homologs of tetx or TETX can be identified in silico by searching one or more publically available sequence databases or experimentally with the well-known molecular biology techniques, for example, polymerase chain reaction (PCR) and hybridization techniques. Certain examples of homologs TETX in certain eukaryotic organisms are provided by Uniprot Entry numbers: Q93L50 (SEQ ID NO: 65), G8SBP9 (SEQ ID NO: 66), Q7X2A0 (SEQ ID NO: 67), E1UR95 (SEQ ID NO: 68), H8MZP2 (SEQ ID NO: 69), R4LB07 (SEQ ID NO: 70), A6GZT8 (SEQ ID NO: 71), B5TTM2 (SEQ ID NO: 72), Q01911 (SEQ ID NO: 73), Q93L51 (SEQ ID NO: 74), E5D2K6 (SEQ ID NO: 75), and A0A0H4JF53 (SEQ ID NO: 76).

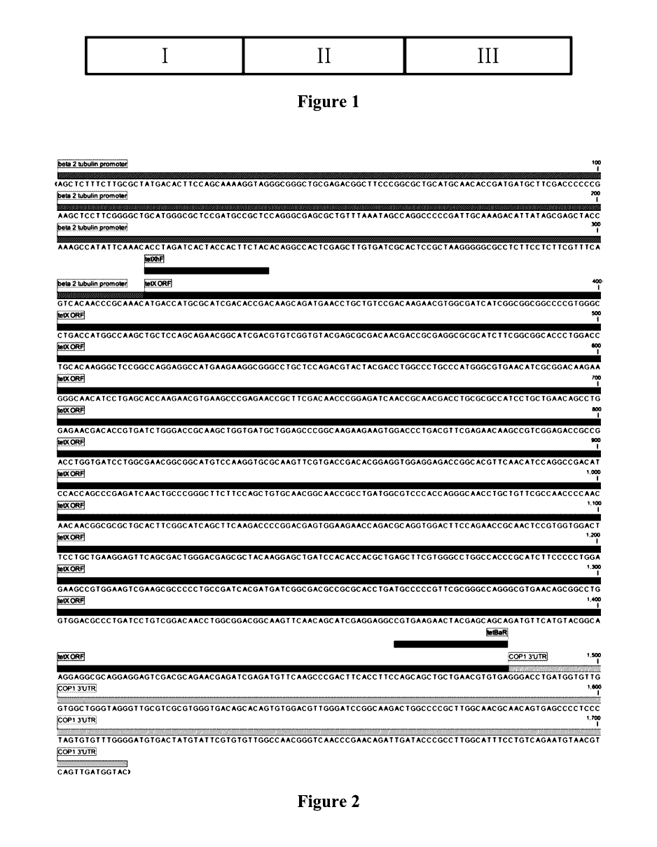

Another embodiment of the invention provides a DNA construct comprising a promoter operably linked to a tetx, for example, a tetx having the sequence of any one of SEQ ID NOs: 1-62, a homolog thereof, or a fragment thereof. In addition to the promoter at the 5′ end of the operably linked gene, one or more sequences that induce transcription of a gene can also be present, for example, in the introns or 3′ end of the operably linked gene (FIG. 1). A further embodiment provides a vector comprising a DNA construct having a promoter operably linked to a tetx and further comprising vector elements, for example, an appropriate origin of replication, a multiple cloning site, a selectable marker gene.

In one embodiment, the promoter is a heterologous promoter. In certain embodiments, the promoter drives the expression of tetx in a eukaryotic cell. Non-limiting examples of a promoter that drives the expression of a tetx in a eukaryotic cell includes a promoter selected from cmv, ef1a, sv40, pgk1, ubc, beta actin, beta 2 tubulin, hsp70a/rbcs2, introns of rbcs2, cag, uas, ac5, polyhedrin, camkIIa, gal1, gal10, tef1, gds, adh1, camv35S, ubi, h1, ponA or u6 promoter.

In one embodiment, the promoter is a constitutive promoter, i.e., a promoter that drives the expression of an operably linked gene in all circumstances. An example of constitutive promoter is beta 2 tubulin promoter. In another embodiment, the promoter is an inducible promoter, for example, PonA promoter that drives the expression of tetx only in the presence of appropriate inducer, namely, ecdysone. Additional examples of constitutive and inducible promoters are known in the art and such embodiments are within the purview of the invention.

A further embodiment provides a DNA construct comprising a first promoter operably linked to a tetx and a second promoter operably linked to a gene of interest. In addition to the promoter at the 5′ end of the operably linked gene of interest, the sequences that induce transcription of a gene can also be present in the introns or 3′ end of the operably linked gene (FIG. 1).

The gene of interest encodes a protein of interest. Proteins of interest are reflective of the commercial markets and interests of those involved in the development of the genetically engineered eukaryotic cell. General categories of proteins of interest include therapeutic proteins, for example, antibodies, fusion proteins; enzymes of commercial interest, for example, amylase or lysozyme. Additional examples of proteins of interest are well known to a person of ordinary skill in the art and such embodiments are within the purview of the invention.

In an embodiment, one or both of the first and the second promoters are heterologous for the genes to which they are operably linked. In a further embodiment, the first and the second promoters are identical or different from each other. In an additional embodiment, one or both of the first and the second promoters are inducible with identical or different inducers. In a particular embodiment, the first promoter is an inducible promoter and the second promoter is a constitutive promoter or vice versa. Examples of promoters discussed above are also relevant to this embodiment.

A further embodiment provides a vector containing the DNA construct comprising a first promoter operably linked to a tetx and a second promoter operably linked to a gene of interest and further comprising vector elements, for example, an appropriate origin of replication, a multiple cloning site, a selectable marker gene. The vector is appropriate for transformation of a eukaryotic cell. Examples of such vector include, but are not limited to, viral vectors, such as adenovirus, retrovirus, vaccinia virus, poxvirus, adeno-associated virus, herpes simplex virus, and lentivirus; and plasmid vectors, such as yeast integrating plasmid, yeast replicating plasmid, yeast centromere plasmid, yeast episomal plasmid, and 2 μm plasmid. Choice of vectors useful for the transformation of a eukaryotic cell depends on the type of eukaryotic cell, for example, a plant cell, a mammalian cell, an insect cell, a yeast cell, a protozoan cell or an algal cell and the purpose of transformation, for example, expression of a protein of interest. Additional examples of vectors suitable for use in a eukaryotic cell are known in the art and such embodiments are within the purview of the invention. In specific embodiments, the eukaryotic cell is C. reinhardtii, S. elongates, or Saccharomyces cerevisiae.

A method of producing a eukaryotic cell comprising a vector, a DNA construct or a polynucleotide molecule described herein is also provided. The method comprises the steps of:
i) introducing a vector, a DNA or a polynucleotide molecule into eukaryotic cells,
ii) culturing the eukaryotic cells in the presence of a tetracycline, and
iii) identifying and isolating the eukaryotic cells that survive and grow in the presence of the tetracycline.

The methods of introducing a vector, a DNA construct, or a polynucleotide molecule into eukaryotic cells and culturing, identifying, and isolating the cells that survive and grow in the presence of a tetracycline are known in the art. For example, the polynucleotide molecules, DNA constructs, or vectors of the invention can be introduced into a eukaryotic cell by an appropriate method of transformation, for example, electroporation, lipofection, microinjection, bio-ballistics, etc. Additional examples of methods of transformation of a eukaryotic cell are known to a person of ordinary skill in the art and such embodiments are within the purview of the invention.

Once a vector, a DNA construct, or a polynucleotide molecule is inside a cell, an incubation period is necessary for the production of TETX. After that incubation period, the cells are typically transferred to a selective media containing one or more tetracyclines such as tetracycline, chlortetracycline, oxytetracycline, demeclocycline, lymecycline, meclocycline, methacycline, minocycline, rolitetracycline, doxycycline, or tigecycline. The purpose of the antibiotics is to kill or reduce the growth of cells that do not contain tetx and do not produce TETX. Cells that grow or display an increased growth in the presence of a tetracycline are transferred to selective media. DNA analysis on a selected cell, such as sequencing, restriction endonuclease cleavage, or polymerase chain reaction, can be used to confirm the presence of the transferred vector, DNA construct or polynucleotide molecule into the cell.

A further embodiment of provides a eukaryotic cell having the vector, a DNA construct, or a polynucleotide molecule comprising tetx. In certain embodiments, the vector, the DNA construct, or the polynucleotide molecule remains in the eukaryotic cell as an extra-chromosomal genetic material and the vector, the DNA construct, or the polynucleotide molecule replicates and propagates into the progeny cells independent from the chromosomes of the eukaryotic cell. In other embodiments, the vector, the DNA construct, or the polynucleotide molecule is incorporated into the chromosome of the eukaryotic cell. Once incorporated into the cell's chromosome, the vector, the DNA construct, or the polynucleotide molecule replicates with the chromosome of the cell and is propagated into the progeny as a part of the cell's chromosomes.

In certain embodiments, the vector, the DNA construct, or the polynucleotide molecule is present in the nucleus of the eukaryotic cell. Typically, proteins encoded by genes expressed in a nucleus of a cell can be post-translationally modified and can be secreted to the exterior of the cells.

A further embodiment of the invention provides a method of producing a eukaryotic cell capable of producing a protein of interest encoded by a gene of interest. The method comprises the steps of:
a) introducing a DNA construct into eukaryotic cells, the DNA construct comprising:
   ii) a first promoter operably linked to a tetx; and
   ii) a second promoter operably linked to a gene of interest;
b) culturing the eukaryotic cells in the presence of a tetracycline, and
c) identifying and isolating the eukaryotic cells that survive and grow in the presence of the tetracycline.

The cells that grow in the presence of tetracycline contain tetx present in the DNA construct and consequently, contain the gene of interest. Therefore, the method further comprises the step of producing the protein of interest by expressing the gene of interest by culturing the eukaryotic cell containing the tetx under conditions that causes the expression of the tetx and the gene of interest. In a further embodiment, the protein of interest is purified from the cultured eukaryotic cells expressing the protein of interest.

The promoters, the genes of interest, the proteins of interest, methods of introducing DNA into a eukaryotic cell, and the other aspects described above in connection with the cell comprising a tetx and a gene of interest are also relevant to this embodiment.

### Materials and Methods

Algal Strain and Growth Conditions

Cell wall deficient strain CC-849 of C. reinhardtii (Chlamydomonas Resource Center, University of Minnesota) was used in all algal transformation experiments. This strain is readily transformed with glass beads or electroporation. Strain CC-124 (Chlamydomonas Resource Center) was used as a control to assay tetracycline sensitivity of WT cell wall strains.

All algal strains were grown routinely in TAP media at 25° C. with a 16/8 light/dark photoperiod in a growth chamber on top of translucid glass shelves. In front of the shelves, two pairs of Sylvania GRO-LUX 40 W wide spectrum fluorescent light tubes (Osram sylvania ltd. Mississauga, ON, CA) and two OCTRON ECO 32 W fluorescent light tubes (Osram sylvania ltd) placed perpendicular to the shelve provided light. Plates were placed at 3 to 40 cm from the light source which provided light from 78.6 to 25 μmoles/sm². Light intensity was measured with a LI-250A light meter (LI-COR, Lincoln, Nebr.), readings are the sum of 15 second averages from two positions: placing the sensor on top of the glass shelf targeted at the ceiling and at the same place with the sensor targeted towards the floor. To achieve lower than 25 μmoles/sm², plates were placed in front of only one pair of Sylvania GRO-LUX 40 W wide spectrum fluorescent light tubes at 30-40 cm from the light source. The lowest light setting: 1.81 μmoles/sm², was achieved by placing the plates in the lowest light location in the growth chamber (17 μmoles/sm²) and covering the plates with two double-layers of gauze.

Plasmid Construction

E. coli strains carrying Plasmids pHsp70A/RbcS2-cgLuc, pSP124S ble cassette and pKS-aphVIII-lox were obtained from the Chlamydomonas Resource Center.

tetx open reading frame [native sequence from Enterobacteriaceae bacterium; Genbank: JQ990987] was synthesized de novo at GenScript (Piscataway, N.J.) with codons optimized for C. reinhardtii cytoplasmic expression under the control of constitutive beta 2 tubulin promoter and chlamyopsinl 3′ UTR; the plasmid was named Btetx (FIG. 2). A second version of the construct was generated by Polymerase Chain Reaction (PCR), amplifying the open reading frame with a Veriti thermal cycler (Applied Biosystems, Foster City, Calif.) in a 25 μL reaction volume containing 1 U of the proof-reading high fidelity (1 error/100,000 bp=0.001%) enzyme Advantage HD DNA Polymerase (Clontech, Palo alto), 0.1 mM dNTP's, and 0.25 μM of each oligo tetXhF and tetBaR (Table 3) that carried XhoI and BamHI sites in their 5′ ends for cloning purposes. The reaction was carried out with an initial denaturation at 98° C. 2 min. following 35 cycles of 98° C. 10 sec, 70° C. 10 sec, 72° C. 1 min. with a final 5 min extension at 72° C. The amplicon was digested with BamHI and XhoI (New England Biolabs, Ipswich, Mass.), and cloned into the corresponding sites of plasmid pHsp70A/RbcS2-cgLuc, replacing the luciferase ORF with that of tetx. The ligation was transformed into E. coli stbl4 (Invitrogen, Carlsbad, Calif.) generating plasmid AtetX. Glass bead transformation C. reinhardtii strain CC-849 was transformed with supercoiled plasmid DNA of Atetx, Btetx, pKS-aphVIII or pSP124S (Table 4) by the glass bead method. Briefly, the algae were grown at 25° C. with a 18/6 (light/dark) photoperiod in TAP media to mid-log phase (1-2×10⁶ cells/mL), the cells were harvested by centrifugation 5 min. at 5000 g, the growth medium was removed and fresh TAP was added to achieve a cell concentration of 2×10⁸ cells/mL. 300 μL of the cell suspension were placed in a 1.5 mL centrifuge tube containing 0.3 g of sterile 0.4-0.6 mm diameter glass beads (Sigma, St. Louis, Mo.) and 1×10⁻¹² mols of the desired plasmid DNA. The cell/DNA/glass bead suspensions were vortexed 15 s at maximum power in a VWR mini vortex. The cells were transferred to a glass tube with 5 mL of fresh TAP media and incubated at 25° C. overnight with a 8/6 (light/dark) photoperiod. After 14 hours the cells were concentrated by centrifugation at 5,000 g for 15 minutes and resuspended in TAP media to yield either 3×10⁷ cells/mL of pKS-aphVIII and pSP124S transformants and 2.5×10⁶, 5×10⁶, 1×10⁷ or 3×10⁷ cells/mL for tetx transformants. 300 μL of the cell suspensions were spread on 100 mm diameter and 15 mm depth TAP agar plates supplemented with either 150 μg/mL paromomycin, 20 μg/mL Zeocin or 15 μg/mL tetracycline. Plates were incubated at 25° C. with a 18/6 (light/dark) photoperiod in low light (1.81 μmoles/sm²) for 1 day, and then tetracycline plates were transferred to medium light (25 μmoles/sm²) while paromomycin and zeocin plates were incubated in high light (78.6 μmoles/sm²). When colonies appeared, they were streaked on to selective TAP agar plates.

TABLE 3
Sequences of the primers

| Name | Sequence |
|---|---|
| tetXhF | TCTCGAGATGACCATGCGCATCGACACCGA |
| | (SEQ ID NO: 81) |
| tetBaR | TGGATCCTCACACGTTCAGCAGCAGCTGCTG |
| | (SEQ ID NO: 82) |
| aph8F | CGTGCACTGCGGGGTCGGT (SEQ ID NO: 83) |
| aph8R | CCGCCCCATCCCACCCGC (SEQ ID NO: 84) |
| ble1F | CCGGGTCGCGCAGGGC (SEQ ID NO: 85) |
| ble1R | GCGCCGTTCCGGTGCTCA (SEQ ID NO: 86) |

PCR Confirmation of Transformants

To verify the gene presence in transformants, colony PCR was performed in a 15 μL reaction volume containing 7.5 μL of GoTaq Green Master Mix (Promega, Madison, Wis.), 0.25 μM of each appropriate oligo pair: aph8F, aph8R; ble1F, ble1R; tetXhF, tetBaR (Table 3). An initial 5 minute denaturation at 95° C. was performed, followed by 35 cycles of 95° C. 20 sec, 60-70° C. 15 sec, 72° C. 1 min with a final 5 min extension at 72° C. The amplicons were resolved in a 1% TAE-agarose gel stained with Sybr safe (Invitrogen, Carlsbad, Calif.). Positive colonies confirmed by PCR were counted and the efficiency reported as colony forming units (cfu) per μg of DNA.

Tetracycline Sensitivity Resistance Assays

Five random strains of BtetX, one of AtetX and one of CC-849 untransformed control were selected from TAP agar plates and 10⁴ cells of each strain were grown in 2 mL TAP media without tetracycline. Positive controls were grown on TAP agar plates supplemented with 15 μg/mL tetracycline. After five days, cell concentration of the cultures was 1×10⁷ cells/mL which corresponds to 26 cell divisions. At that time, cultures of strains grown with or without antibiotic were diluted with TAP media to 1×10⁶ cells/mL, and 10 μL (10⁴ cells) of each strain were grown on TAP agar plates containing tetracycline at 15, 25, 50 and 100 μg/μL. After 7 days of growth at a light intensity of 25 moles/sm² photographs were taken of each plate.

Wild Type C. reinhardtii Tetracycline Sensitivity

C. reinhardtii strain CC-124 (wt, mt-) was grown in TAP agar supplemented with tetracycline at concentrations of 15, 25 or 50 μg/mL, and cell concentrations from 0.5, 1.0, 2.5 and 5.0 million cells per plate, incubated for 10 days at a light intensity of 25 μmoles/sm².

Tetx Plasmid Deposit

The TetXA and TetXB transformation plasmids have been deposited with the Chlamydomonas Resource Center, University of Minnesota.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1

### Transformation of C. reinhardtii Using TetX

A cell wall deficient strain for transformation and selection was used. The sensitivity of this strain to tetracycline was checked using a wild type (WT) cell-walled strain, CC-124, to evaluate more general utility. Strain CC-124 was grown in TAP agar plates, under the same light conditions used for transformation, tetracycline concentrations of 15, 25 and 50 μg/mL, and cell concentrations from 0.5 1.0, 2.5 and 5.0×10⁶ cells per plate. After a period of 10 days, the time required for transformants to become visible, no growth of wild type cells was observed. Thus, tetracycline uptake at the assayed concentrations was not sufficiently affected by the wild type cell-wall to alter the sensitivity of the cells to tetracycline.

tetx open reading frame (SEQ ID NO: 3) was synthesized de novo at GenScript (Piscataway, N.J.) with codons optimized for C. reinhardtii cytoplasmic expression under the control of constitutive beta 2 tubulin promoter. The plasmid was named BtetX (FIG. 2). A second version of the construct was generated by PCR, amplifying the open reading frame with a Veriti thermal cycler (Applied Biosystems, Foster City, Calif.) in a 25 μL reaction volume containing 1 U of the proof-reading high fidelity (1 error/100,000 bp=0.001%) enzyme Advantage HD DNA Polymerase (Clontech, Palo alto), 0.1 mM dNTP's, and 0.25 μM of each oligo tetXhF 5′-TCTCG AGATG ACCAT GCGCA TCGAC ACCGA (SEQ ID NO: 81) and tetBaR 5′-TGGAT CCTCA CACG TTCAG CAGCT GCTG (SEQ ID NO: 82) that carried XhoI and BamHI sites in their 5′ ends for cloning purposes. The reaction was carried out with an initial denaturation at 98° C. 2 minutes, following 35 cycles of 98° C. 10 sec, 70° C. 10 sec, 72° C. 1 minute with a final 5 min extension at 72° C. The amplicon was digested with BamHI and XhoI (New England Biolabs, Ipswich, Mass.), and cloned into the corresponding sites of plasmid pHsp70A/RbcS2-cgLuc, replacing the luciferase ORF with that of tetx. The ligation was transformed into E. coli stbl4 (Invitrogen, Carlsbad, Calif.) generating plasmid AtetX.

C. reinhardtii strain CC-849 was transformed with supercoiled plasmid DNA of AtetX and BtetX, by the glass bead method. Briefly, the algae were grown at 25° C. with a 18/6 (light/dark) photoperiod in TAP media to mid-log phase (1-2×10⁶ cells/mL), the cells were harvested by centrifugation for 5 minute at 5000 g, the growth medium was removed and fresh TAP was added to achieve a cell concentration of 2×10⁸ cells/mL. 300 μL of the cell suspension was placed in a 1.5 mL centrifuge tube containing 0.3 g of sterile 0.4-0.6 mm diameter glass beads (Sigma, St. Louis, Mo.) and 1×10⁻¹² moles of the desired plasmid DNA. The cell/DNA/glass bead suspensions were vortexed for 15 s at maximum power in a VWR mini vortex. The cells were transferred to a glass tube with 5 mL of fresh TAP media and incubated at 25° C. overnight with a 8/6 (light/dark) photoperiod. After 14 hours, the cells were concentrated by centrifugation at 5,000 g for 15 minutes and resuspended in TAP media to yield 2.5×10⁶, 5×10⁶, 1×10⁷ or 3×10⁷ cells/mL. 300 μL of the cell suspensions were spread on 100 mm diameter and 15 mm depth TAP agar plates supplemented with 15 μg/mL tetracycline. Plates were incubated at 25° C. with a 18/6 (light/dark) photoperiod in low light (1.81 moles/sm²) for 1 day, and then tetracycline plates were transferred to medium light (25 moles/sm²) while paromomycin and zeocin plates were incubated in high light (78.6 moles/sm²). When colonies appeared, they were streaked on to selective TAP agar plates.

TABLE 4
Listing of plasmids used in this example with their corresponding promoter/terminator/number
of RBCS2 introns, associated resistance, plasmid size, molecular weight
(MW), transformation efficiency (TE), and source.

| | Promoter/terminator/#R | | Base pairs/ | | |
|---|---|---|---|---|---|
| Plasmid | BCS2 introns | Resistance | MW (g/mol) | TE | Source |
| AtetX | HSP70A:RBCS2/RBCS2/1 | Tetracycline | 5238/ | 6.18 | This |
| | | | 3236640.1 | | example |
| BtetX β-2 | tubulin/COP-1/NO | Tetracycline | 4445/ | 3.28 | This |
| | | | 2746642.9 | | example |
| pKs- | β-2 tubulin/COP-1/NO | Paromomycin | 4308/ | 4.51 | Heitzer et |
| aphVIII | | | 2661961.7 | | al. (2007) |
| psP124S-ble | RBCS2/RBCS2/2 | Zeocin | 4133/ | 22.56 | Lumbreras |
| | | | 2553770 | | et al. (1998) |

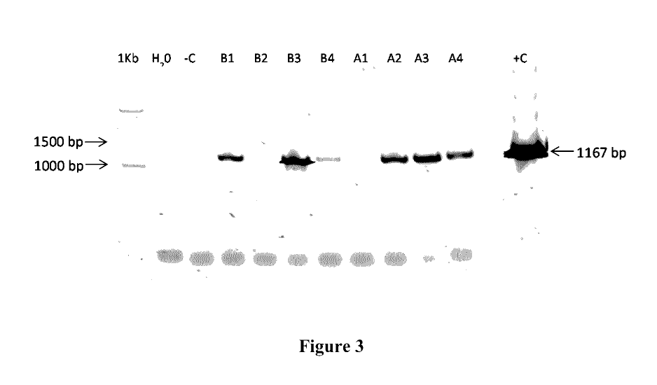

To verify the presence of tetx in transformants, colony PCR was performed in a 15 μL reaction volume containing 7.5 μL of GoTaq Green Master Mix (Promega, Madison Wis.), 0.25 μM of each appropriate oligo pair: tetXhF and tetBaR. An initial 5 minute denaturation at 95° C. was performed, followed by 35 cycles of 95° C. 20 sec, 60-70° C. 15 sec, 72° C. 1 minute with a final 5 minute extension at 72° C. The amplicons were resolved in a 1% TAE-agarose gel stained with Sybr safe (Invitrogen, Carlsbad, Calif.). The gel is provided in FIG. 3.

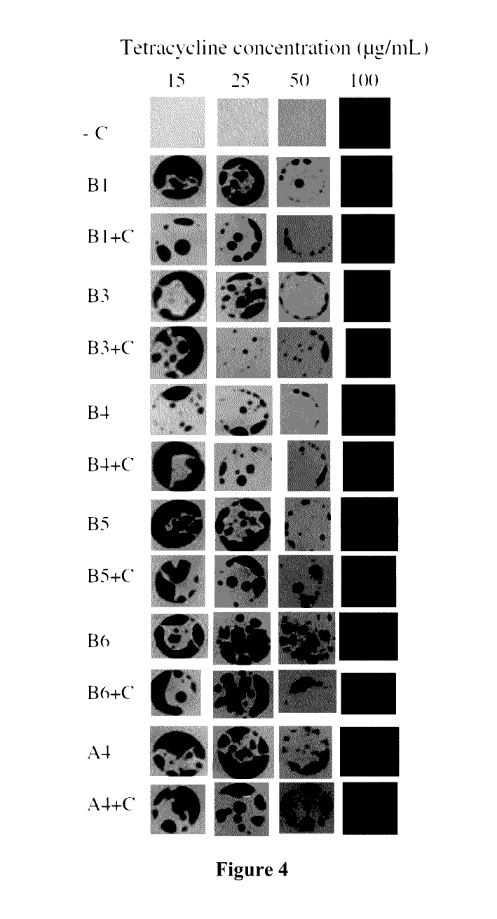

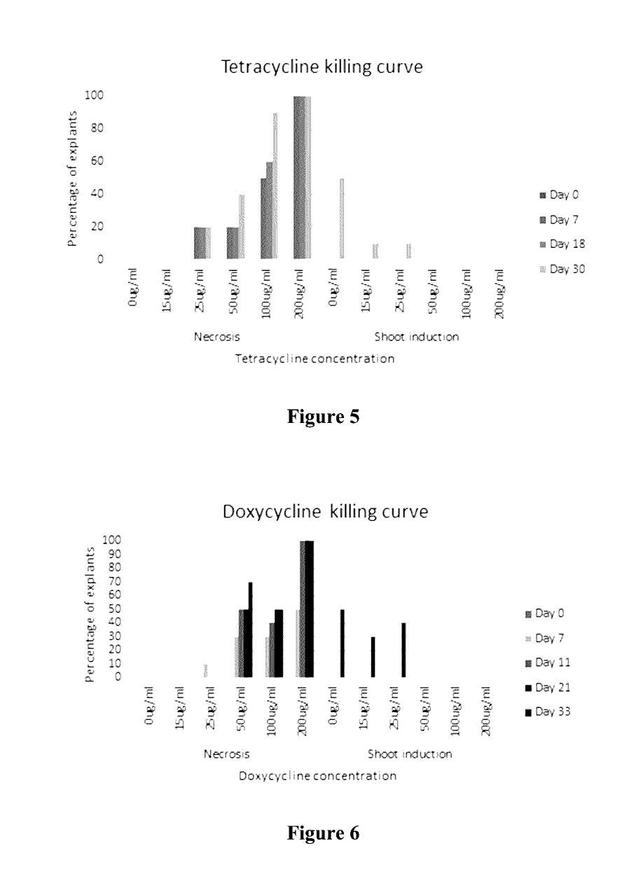

Transformations with both constructs were carried out and, 8-12 days after plating transformed cells, tetracycline resistant C. reinhardtii colonies appeared. Both promoters used to drive tetx expression yielded transformant colonies, which indicates the versatility of this system to work with low and high level expression promoters (FIG. 4).

As light causes tetracycline degradation, the relationship between the appearance of resistant positive or false positive colonies with respect to cell concentration per plate and light intensity (Table 5). When 5×10⁶ cells/plate were grown under medium light conditions (17-25 moles/sm²), tetracycline at 15 μg/mL was sufficient to prevent false positives from growing on selection plates. All transformants selected at these conditions were positive. However at concentrations above 5×10⁶ cells/plate, and/or light intensity above 27 moles/sm² false positives appeared as either a lawn or patches of small colonies. However because positive colonies grow faster than negative transformants, they can be easily distinguished and selected from the small colony false were positive. Positive transformants can be grown on plates containing tetracycline concentrations up to 100 μg/mL. Tetracycline concentrations of 25 and 50 μg/mL were analyzed to select primary transformants; however, at those concentrations the number of positive colonies decreased approximately 75% compared to those obtained with 15 μg/mL.

TABLE 5
Effect of light intensity and cell concentration
on false positive appearance.

| | Light intensity (μmoles/sm²) | | |
|---|---|---|---|
| Cells/plate | 17 | 24 | >26 |
| 2.5 × 10⁶ | − | − | + |
| 5.0 × 10⁶ | − | − | + |
| 1.0 × 10⁶ | − | + | + |
| 3.0 × 10⁶ | + | + | + |

Tetracycline resistance was assayed when positive transformed strains expressing the tetx were grown without antibiotic (FIG. 4). Antibiotic resistance was maintained at minimum for 26 divisions, and transformants were also resistant to concentrations up to 100 μg/mL (FIG. 4).

A new stable nuclear selection marker for C. reinhardtii that confers resistance to tetracycline at up to 100 μg/mL is provided. As TETX hydrolyzes several tetracycline analogues, their use might favor increased light incubation to obtain transformants. Thus tetx produces a versatile tetracycline degrading enzyme, which can be used to transform the nucleus of other microalgae, as well as the chloroplast or mitochondria of other tetracycline sensitive cells, such as S. cerevisiae or human HeLa cells that are sensitive to tetracycline concentrations above 10 μg/mL. Codon bias of the specific target host and organelle can be considered to optimize expression in a given cell.

### Example 2

### Transformation of Dunaliella salina

The marine eukaryotic microalgae D. salina was transformed with natural or synthetic DNA encoding the tetx under the control of regulatory elements appropriate for use in this algal system. The gene was inserted in D. salina by particle bombardment. Following an incubation period, cells were cultured in growth media containing a tetracycline for direct selection of positive transformants. After 8-12 days, visible colonies were re-streaked and colony PCR was carried out to verify the presence of introduced DNA in putative transformants.

### Example 3

### Transformation of Plants and Plant Cells

Many different plant tissues and plant cells from a variety of plant species have been shown to be transformable using a number of different transformation systems, such as Agrobacterium mediated transformation, particle bombardment, etc. The particular type of plant tissue or plant cell may require that certain systems be used. Although some specific plant tissues are described, a person of ordinary skill in the art can extrapolate this example to any transformable plant tissues using mechanisms of plant or plant cell transformation known in the art.

In the case of whole plants, regeneration of transformed tissue may be accomplished via a callus intermediate or through direct organogenesis. For example, potato explants can be transformed by using DNA construct comprising tetx under the control of an appropriate plant promoter contained on a binary vector via standard Agrobacterium mediated transformation. Alternately, stem internode segments 0.5-1 cm in length can be excised from 6 week old plants and inoculated the same day with A. tumefaciens containing a binary vector comprising the tetx under the control of appropriate plant promoter via standard Agrobacterium mediated transformation. Approximately 100 stem internode explants can be incubated per 50 ml of inoculum for 10 minutes agitating occasionally and blotted on to sterile filer paper and plated on to MS medium with appropriate additives. After 48 hours the explants can be transferred to MS medium with the appropriate additives plus a tetracycline at an appropriate concentration. Alternatively, a second selectable marker can be used (e.g. bialophos or kanamycin resistance) and the primary transformants selected for resistance to the identified secondary selectable marker and then screened for resistance to a tetracycline. PCR, or an appropriate alternative, can be performed to verify the presence of the incorporated DNA in the putative transformants. Whole tetracycline resistant transformed plants can be regenerated using standard procedures.

Tobacco BY-2 cells can be transformed by using DNA construct encoding tetx under the control of an appropriate plant promoter contained on a binary vector via standard Agrobacterium mediated transformation. Alternatively, NT-1 BY-2 tobacco cells or other plant tissues can be directly transformed via particle bombardment with a DNA construct containing a plant expression cassette with appropriate plant promoters operably linked to a tetx. Other forms of plant transformation may also be used. Following an incubation period, cells can be cultured in growth media containing a tetracycline for direct selection of transformants. PCR, or an appropriate alternative, can be performed to verify the presence of the incorporated DNA in putative transformants.

Sensitivity to a tetracycline can be evaluated for the specific explant tissue used for transformation and regeneration. To this end, the sensitivity of Tobacco leaf tissue and Jalapeño hypocotyl, epicotyl and shoot tip tissues towards selected concentrations of tetracycline and doxycycline was evaluated. Tetracycline and doxycycline stock solution were freshly prepared in sterile distilled water at 100 mg/ml concentration and 0.22 μm filter sterilized before being diluted into media. Explants were placed on MS solid medium supplemented with appropriate hormones and varying concentrations of either tetracycline or doxycycline at: 0, 5, 10, 15, 25, 50, 100, 150 and 200 μg/mL. After 7 days, 100% of the tobacco explants exhibited necrosis at a 200 μg/mL tetracycline concentration and at the same time shoot induction was inhibited. After 30 days, shoot induction was observed in 50% of the control plates and plates containing 15 and 25 μg/mL tetracycline but not for the other tested concentrations. After 7 days, 100% of the tobacco explants exhibited necrosis at a 200 μg/mL doxycycline concentration and at the same time shoot induction was inhibited. After 33 days, shoot induction was observed in control plates and plates containing 15 and 25 μg/mL doxycycline but not for the other tested concentrations. Similar effects were seen with both tetracycline and doxycycline with the Jalapeño explants, initially evaluated after 15 days. Controls survival, as exhibited by limited growth (principally callus formation), could be discerned on plates containing up to 100 μg/mL of either tetracycline or doxycycline, but 100% exhibited necrosis at 200 μg/mL.

An expression cassette for plant transformation was developed with a gene encoding TETX optimized for plant expression under the control of a plant expressible promoter (CaMV35S) and a 3′ polyA region (VSP). This cassette can be used for particle bombardment or inserted in a binary vector for Agrobacterium mediated plant transformation. A Binary Vector was constructed with this TETX expression cassette that also carried the bar gene under the control of plant expression regulatory sequences (from Nopaline Synthase) to permit the options of selection with phosphinothricin and screening with tetracycline or doxycycline, or the direct selection with a tetracycline. This construct is used for the transformation of explant tissues demonstrating that expression of tetx can be used for the selection of positive transformants on media containing appropriate concentrations of a tetracycline.

Example 4. Transformation of yeast cells Sensitivity of the yeast strains, namely, Kluyvermyces lactis, Kluyveromyces marxianus, Pichia pastoris and Saccharomyces cerevisiae ATCC 44774, to tetracycline and doxycycline was analyzed in agar media for the selection of genetic transformants. From a single overnight grown colony of each yeast strain, 25 mL of yeast extract-peptone-dextrose (YPD) broth were inoculated and grown for 16 hours at 30° C. and 150 rpm. Cell concentration was estimated by cell counting and 10⁵, 10⁶, or 10⁷ cells were plated on yeast extract-peptone-glycerol-ethanol (YPGE) agar petri dishes containing tetracycline or doxycycline up to 1000 μg/mL.

After 2 days of incubation K. lactis growth was inhibited with tetracycline, at 750 and 1000 μg/mL at for 10⁵ cells and 500, 750, and 1000 μg/mL tetracycline inhibited S. cerevisiae. K. marxianus growth was also inhibited from 350-1000 μg/mL at 10⁵ cells and 1000 μg/mL at 10⁶. P. pastoris growth was unaffected by tetracycline at the assayed concentrations.

Doxycycline inhibited growth of K. lactis and K. marxianus at 10⁶ cells plated with 675 and 1000 μg/mL and at 10⁷ with 1000 μg/mL. Unlike tetracycline, doxycycline inhibited growth of P. pastoris at 10⁶ and 1000 μg/mL. S. cerevisiae growth was inhibited at 10⁵ and 10⁷ at a concentration of 1000 μg/mL.

Transformation competent yeast cells were prepared using a method specific for each strain. For K. lactis, the method of Rajagopal et al.; for K. marxianus, the method of Abdel-Banat et al.; for P. pastoris, the method of Wu et al.; and for S. cerevisiae, the method of Gietz et al. was used.

Following protocols recommendations, from 50 ng to 1 μg of Linearized plasmid DNA containing the tetx codon optimized for yeast expression was electroporated or introduced through heat-shock to the yeast cells. After 3 hours of recovery from the transformation, yeast cells can be plated in YPGE agar plates supplemented with 1000 μg/mL of doxycycline, after 2-4 days positive transformant colonies were visible in the plates. The colonies were selected and screened to confirm the transformation. Specific primers for tetx amplification using polymerase chain reaction can be used. Alternatively a gene reporter can also be used such as a fluorescent protein, and the positive transformants can be assayed for fluorescence.

### Example 5

### Transformation of Insect Cells

Sf21 cells from Spodoptera frugiperda are transformed using a DNA construct containing a tetx under the control of appropriate insect cell promoter. A plasmid containing the insect cell promoter and a tetx is electroporated into Sf21 cells. Following an incubation period, the cells can be cultured in growth media containing a tetracycline for direct selection of transformants. PCR can be performed to verify the presence of the incorporated DNA in putative transformants.

### Example 6

### Transformation of Chinese Hamster Ovary (CHO) Cells

CHO cells are commonly used to produce recombinant biopharmaceutical proteins, mainly antibodies. Genetic transformation is usually carried out via transfection using a lipid carrier containing DNA. TransIT-PRO transfection kit was used to obtain tetx transformant CHO cells.

CHO cells were grown in FreeStyle media (Invitrogen) and inoculated at 10⁵ cells/mL in 2/3 of the total transfection volume. On the day of transfection, cell number was determined and cells were diluted in FreeStyle media without antibiotic to achieve a concentration of 5×10⁵ cells/mL. For a 30 mL flask transfection, 3 mL OptiPro, 30 μg of tetx CHO codon optimized DNA, 15 μL transIT PRO and 30 μL PRO Boost were mixed and incubated at room temperature for 25 minutes.

The transfection solution was added slowly to the culture flask, cells were incubated with slow shaking for 48 hours. Cell concentration and viability was determined by counting in a New Bauer cell counting system with trypan blue staining. Cells were collected by centrifugation and suspended in fresh media with 5-20 μg/mL tetracycline, doxycycline or tigecycline to a cell density of 10⁵ cells/mL.

Cells were incubated at 37° C., 70-80% relative humidity and 5-8% CO₂ without agitation. On day 7 post-transfection, cells were transferred to a flask at a concentration of 3×10⁵ cells/mL and incubated at 37° C., 70-80% relative humidity and 5-8% CO₂ and 125 rpm. Every 3-4 days, fresh media was be replaced in the culture flasks and 3×10⁵ viable cells/mL were inoculated. When viability was >85% and viable cells are above 10⁶ cells/mL the selection phase was concluded and PCR confirmed positive transformants from the cell pool.

### REFERENCES

1. Cardineau, Guy A., Mason, Hugh Stanley, VanEck, Joyce M., Kirk, Dwayne D., Walmsley, Amanda Maree. U.S. Pat. No. 7,132,291 (Nov. 7, 2006) and U.S. Pat. No. 7,407,802 (Aug. 5, 2008), “Vectors and cells for preparing immunoprotective compositions derived from transgenic plants”, Dow Agro Sciences LLC (Indianapolis, Ind.) and Boyce Thompson Institute for Plant Research (Ithaca, N.Y.)
2. Chang, H. R., Comte, R., & Pechère, J. C. (1990). In vitro and in vivo effects of doxycycline on Toxoplasma gondii. Antimicrobial Agents and Chemotherapy, 34(5), 775-80.
3. Cohen, S. N. (2013). DNA cloning: a personal view after 40 years. Proceedings of the National Academy of Sciences of the United States of America, 110(39), 15521-9.
4. Cohen, S. N., Chang, A. C., Boyer, H. W., & Helling, R. B. (1973). Construction of biologically functional bacterial plasmids in vitro. Proceedings of the National Academy of Sciences of the United States of America, 70(11), 3240-4.
5. Cohen, S. N., Chang, A. C., & Hsu, L. (1972). Nonchromosomal antibiotic resistance in bacteria: genetic transformation of Escherichia coli by R-factor DNA. Proceedings of the National Academy of Sciences of the United States of America, 69(8), 2110-4.
6. Griffiths, A. J., Miller, J. H., Suzuki, D. T., Lewontin, R. C., & Gelbart, W. M. (2000). Recombinant DNA technology in eukaryotes (7th ed.). W. H. Freeman.
7. Ku, T. S. N., Palanisamy, S. K. A., & Lee, S. A. (2010). Susceptibility of Candida albicans biofilms to azithromycin, tigecycline and vancomycin and the interaction between tigecycline and antifungals. International Journal of Antimicrobial Agents, 36(5), 441-6. doi:10.1016/j.ijantimicag.2010.06.034
8. Lavarde, V., Acar, J. F., & Drouhet, E. (1975). [Effect of minocycline on Candida albicans. “In vitro” study: comparison with tetracycline]. Pathologie-Biologie, 23(9), 725-8.
9. Moore, I. F., Hughes, D. W., & Wright, G. D. (2005). Tigecycline is modified by the flavin-dependent monooxygenase TetX. Biochemistry, 44(35), 11829-35. doi:10.1021/bi0506066
10. Mulsant, P., Gatignol, A., Dalens, M., & Tiraby, G. (1988). Phleomycin resistance as a dominant selectable marker in CHO cells. Somatic Cell and Molecular Genetics, 14(3), 243-52.
11. Sambrook, J., & Russell, D. W. (2001). Molecular cloning: a laboratory manual. 3rd ed. Cold Spring Harbor Laboratory Press. Cold Spring Harbor Laboratory Press.
12. Vile, R. (1992). Selectable markers for eukaryotic cells. Methods in Molecular Biology (Clifton, N.J.), 8, 49-60. doi:10.1385/0-89603-191-8:49
13. Waterworth, P. M. (1974). The effect of minocycline on Candida albicans. Journal of Clinical Pathology, 27(4), 269-72.
14. Yang, W., Moore, I. F., Koteva, K. P., Bareich, D. C., Hughes, D. W., & Wright, G. D. (2004). TetX is a flavin-dependent monooxygenase conferring resistance to tetracycline antibiotics. The Journal of Biological Chemistry, 279(50), 52346-52. doi: 10. 1074/jbc.M409573200
15. Specht E, Miyake-Stoner S, Mayfield S P. Micro-algae come of age as a platform for recombinant protein production. Biotechnol Lett. 2010; 32:1373-83.
16. Cerutti H, Johnson A M, Gillham N W, Boynton J E. Epigenetic silencing of a foreign gene in nuclear transformants of Chlamydomonas. Plant Cell. 1997; 9:925-45.
17. Rasala B A, Lee P A, Shen Z, Briggs S P, Mendez M, Mayfield S P. Robust Expression and Secretion of Xylanase1 in Chlamydomonas reinhardtii by Fusion to a Selection Gene and Processing with the FMDV 2A Peptide. PLoS One. 2012; 7:e43349.
18. Eichler-Stahlberg A, Weisheit W, Ruecker O, Heitzer M. Strategies to facilitate transgene expression in Chlamydomonas reinhardtii. Planta. 2009; 229:873-83.
19. Lumbreras V, Stevens D, Purton S. Efficient foreign gene expression in Chlamydomonas reinhardtii mediated by an endogenous intron. Plant J. 1998; 14(February):441-7.
20. Fuhrmann M, Oertel W, Hegemann P. A synthetic gene coding for the green fluorescent protein (GFP) is a versatile reporter in Chlamydomonas reinhardtii. Plant J. 1999; 19:353-61.
21. Fuhrmann M, Hausherr A, Ferbitz L, Schödl T, Heitzer M, Hegemann P. Monitoring dynamic expression of nuclear genes in Chlamydomonas reinhardtii by using a synthetic luciferase reporter gene. Plant Mol Biol. 2004; 55:869-81.
22. Rasala B A, Barrera D J, Ng J, Plucinak T M, Rosenberg J N, Weeks D P, et al. Expanding the spectral palette of fluorescent proteins for the green microalga Chlamydomonas reinhardtii. Plant J. 2013; 74:545-56.
23. Debuchy R, Purton S, Rochaix J D. The argininosuccinate lyase gene of Chlamydomonas reinhardtii: an important tool for nuclear transformation and for correlating the genetic and molecular maps of the ARG7 locus. EMBO J. 1989; 8:2803-9.
24. Berthold P, Schmitt R, Mages W. An Engineered Streptomyces hygroscopicus aph 7″Gene Mediates Dominant Resistance against Hygromycin B in Chlamydomonas reinhardtii. Protist. 2002; 153(December):401-12.
25. Sizova I, Fuhrmann M, Hegemann P. A Streptomyces rimosus aphVIII gene coding for a new type phosphotransferase provides stable antibiotic resistance to Chlamydomonas reinhardtii. Gene. 2001; 277:221-9.
26. Blochlinger K, Diggelmann H. Hygromycin B phosphotransferase as a selectable marker for DNA transfer experiments with higher eucaryotic cells. Mol Cell Biol. 1984; 4:2929-31.
27. Diaz-Santos E, de la Vega M, Vila M, Vigara J, León R. Efficiency of different heterologous promoters in the unicellular microalga Chlamydomonas reinhardtii. Biotechnol Prog. 2013; 29:319-28.
28. Stevens D R, Rochaix J D, Purton S. The bacterial phleomycin resistance gene ble as a dominant selectable marker in Chlamydomonas. Mol Gen Genet. 1996; 251:23-30.
29. Van der Grinten E, Pikkemaat M G, van den Brandhof E-J, Stroomberg G J, Kraak M H S. Comparing the sensitivity of algal, cyanobacterial and bacterial bioassays to different groups of antibiotics. Chemosphere. 2010; 80:1-6.
30. González-Pleiter M, Gonzalo S, Rodea-Palomares I, Leganes F, Rosal R, Boltes K, et al. Toxicity of five antibiotics and their mixtures towards photosynthetic aquatic organisms: implications for environmental risk assessment. Water Res. 2013; 47:2050-64.
31. Li X-Z, Nikaido H. Efflux-mediated drug resistance in bacteria. Drugs. 2004; 64:159-204.
32. Dönhöfer A, Franckenberg S, Wickles S, Berninghausen O, Beckmann R, Wilson D N. Structural basis for TetM-mediated tetracycline resistance. Proc Natl Acad Sci USA. 2012; 109:16900-5.
33. Yu Z, Reichheld S E, Cuthbertson L, Nodwell J R, Davidson A R. Characterization of tetracycline modifying enzymes using a sensitive in vivo reporter system. BMC Biochem. 2010; 11:34.
34. Chopra I, Roberts M. Tetracycline Antibiotics: Mode of Action, Applications, Molecular Biology, and Epidemiology of Bacterial Resistance Tetracycline Antibiotics: Mode of Action, Applications, Molecular Biology, and Epidemiology of Bacterial Resistance. Microbiol Mol Biol Rev. 2001; 65:232-60.
35. Guiney D G, Hasegawa P, Davis C E. Expression in Escherichia coli of cryptic tetracycline resistance genes from bacteroides R plasmids. Plasmid. 1984; 11:248-52.
36. Speer B S, Bedzyk L, Salyers A A. Evidence that a novel tetracycline resistance gene found on two Bacteroides transposons encodes an NADP-requiring oxidoreductase. J Bacteriol. 1991; 173:176-83.
37. Chang H R, Comte R, Pechère JC. In vitro and in vivo effects of doxycycline on Toxoplasma gondii. Antimicrob Agents Chemother. 1990; 34:775-80.
38. Ku T S N, Palanisamy S K A, Lee S A. Susceptibility of Candida albicans biofilms to azithromycin, tigecycline and vancomycin and the interaction between tigecycline and antifungals. Int J Antimicrob Agents. 2010; 36:441-6.
39. Davies J P, Weeks D P, Grossman A R. Expression of the arylsulfatase gene from the β2-tubulin promoter in Chlamydomonas reinhardtii. Nucleic Acids Res. 1992; 20:2959-65.
40. Cerutti H, Johnson A M, Gillham N W, Boynton J E. A Eubacterial Gene Conferring Spectinomycin Resistance on. Genetics. 1997; 145:97-110.
41. Rose W E, Rybak M J. Tigecycline: first of a new class of antimicrobial agents. Pharmacotherapy. 2006; 26:1099-110.
42. Gari E, Piedrafita L, Aldea M, Herrero E. A set of vectors with a tetracycline-regulatable promoter system for modulated gene expression in Saccharomyces cerevisiae. Yeast. 1997; 13:837-48.
43. Gossen M, Bujard H. Tight control of gene expression in mammalian cells by tetracycline-responsive promoters. Proc Natl Acad Sci USA. 1992; 89:5547-51.
44. Harris E H. The Chlamydomonas Sourcebook. A Comprehensive Guide to Biology and Laboratory Use. San Diego, Calif.: Academic Press. 1989. xiv, 780 pp., illus.
45. Ruecker O, Zillner K, Groebner-Ferreira R, Heitzer M. Gaussia-luciferase as a sensitive reporter gene for monitoring promoter activity in the nucleus of the green alga Chlamydomonas reinhardtii. Mol Genet Genomics. 2008; 280:153-62.
46. Heitzer M, Zschoernig B. Construction of modular tandem expression vectors for the green alga Chlamydomonas reinhardtii using the Cre/lox-system. Biotechniques. 2007; 43:324-32.
47. Kindle K L. High-frequency nuclear transformation of Chlamydomonas reinhardtii. Proc Natl Acad Sci USA. 1990; 87:1228-32.
48. Cao M, Fu Y, Guo Y, Pan J. Chlamydomonas (Chlorophyceae) colony PCR. Protoplasma. 2009; 235:107-10.
49. Rajagopal et al., Meta Gene 2 (2014) 807-818.
50. Abdel-Banat et al., Yeast (2010); 27: 29-39.
51. Wu et al., BioTechniques (2004) 36:152-154.
52. Gietz et al., Nature Protocols (2007) 2; 31-34.

What is claimed is:

1. A DNA construct comprising a first promoter operably linked to a tetx, wherein tetx is a polynucleotide comprising:
(a) the sequence set forth in any one of SEQ ID NOs: 1-62;
(b) a sequence having at least 70% sequence identity to the sequence set forth in any one of SEQ ID NOs: 1-62,
wherein the first promoter drives transcription of the tetx when the DNA construct is in a eukaryotic cell.

2. The DNA construct of claim 1, wherein the first promoter is selected from cmv, ef1a, sv40, pgk1, ubc, beta actin, beta 2 tubulin, hsp70a/rbcs2, introns of rbcs2, cag, uas, ac5, polyhedrin, camkIIa, gal1, gal10, tef1, gds, adh1, camv35S, ubi, h1, ponA or u6 promoter.

3. The DNA construct of claim 1, wherein the first promoter is selected from beta 2 tubulin or hsp70a/rbcs2 promoter.

4. A eukaryotic expression vector comprising the DNA construct of claim 1.

5. A eukaryotic cell comprising the DNA construct of claim 1.

6. The eukaryotic cell of claim 5, wherein the DNA construct is incorporated into a chromosome of the cell.

7. The eukaryotic cell of claim 6, wherein the eukaryotic cell is a plant cell, an animal cell, a yeast cell, a protozoan cell, or an algal cell.

8. The eukaryotic cell of claim 6, wherein the algal cell is Chlamydomonas reinhardtii or Synechococcus elongates.

9. The DNA construct of claim 1, the DNA construct further comprising a second promoter operably linked to a gene of interest.

10. A eukaryotic expression vector comprising the DNA construct of claim 9.

11. A eukaryotic cell comprising the DNA construct of claim 9.

12. A method for expressing a gene of interest encoding a protein of interest in a eukaryotic cell, the method comprising:
a) introducing a DNA construct into the eukaryotic cell, the DNA construct comprising:
ii) a first promoter operably linked to a tetx, wherein the tetx is a polynucleotide comprising the sequence of any one of SEQ ID NOs: 1-62; or a sequence having at least 70% sequence identity to the sequence set forth in any one of SEQ ID NOs: 1-62; and
ii) a second promoter operably linked to the gene of interest; and
b) culturing the eukaryotic cell under conditions that causes the expression of the tetx and the gene of interest.

13. The method of claim 12, the method further comprising isolating the protein of interest.

14. A polynucleotide molecule having the sequence of any one of SEQ ID NOs: 2-62.

15. A DNA construct comprising a first promoter operably linked to the polynucleotide molecule of claim 14.

16. The DNA construct of claim 15, the DNA construct further comprising a second promoter operably linked to a gene of interest.

17. A eukaryotic expression vector comprising the DNA construct of claim 16.

18. A eukaryotic cell comprising the polynucleotide molecule of claim 14.

19. The eukaryotic cell of claim 18, wherein the polynucleotide molecule is incorporated into a chromosome of the cell.

20. The eukaryotic cell of claim 18, wherein the algal cell is Chlamydomonas reinhardtii or Synechococcus elongates.
